(19) United States
(12) Patent Application Publication
(10) Pub. No.: US 2011/0251258 A1
(43) Pub. Date: Oct. 13, 2011

(54) RNAI CONSTRUCTS AND USES THEREOF

(75) Inventors: Dmitry Samarsky, Westborough, MA (US); Tod M. Woolf, Sudbury, MA (US); William Solomon, Worcester, MA (US); Joanne Kamens, Newton, MA (US); Anastasia Khvorova, Northborough, MA (US); Pamela A. Pavco, Longmont, CO (US)

(73) Assignee: RXi Pharmaceuticals Corporation, Worcester (MA)

(21) Appl. No.: 13/055,617

(22) PCT Filed: Jul. 23, 2009
(86) PCT No.: PCT/US09/04326

§ 371 Date: May 31, 2011

Related U.S. Application Data
(60) Provisional application No. 61/135,855, filed on Jul. 24, 2008.
(60) Provisional application No. 61/197,768, filed on Oct. 30, 2008.
(60) Provisional application No. 61/208,394, filed on Feb. 23, 2009.
(60) Provisional application No. 61/209,429, filed on Mar. 6, 2009.

(51) Int. Cl. A61K 31/713; C07H 21/00

(52) U.S. Cl. 514/44 A; 536/24.5

(57) ABSTRACT

The invention relates to improved double-stranded RNAi constructs (sometimes referred to as “solo-rxRNA”) and uses thereof. The construct comprises a structure formed in some aspects of the invention by two identical single-stranded polynucleotides, with the structure having two double-stranded stem regions (each having less than 21 base pairs) and a loop or bulge having about 4 to 11 nucleotides on each strand. The construct is resistant to cleavage by Dicer or other Dicer-like RNase III enzymes and is capable of being loaded into a RISC complex to effect RNA interference. In addition, the nucleotides of the present hairpin constructs may be modified to greatly enhance functionality, such as stability and specificity.

### REFERENCE TO RELATED APPLICATIONS

This application claims priority under 35 U.S.C. §119(e) to U.S. Provisional Application Ser. No. 61/135,855, filed on Jul. 24, 2008; U.S. Provisional Application Ser. No. 61/197,768, filed on Oct. 30, 2008; the U.S. Provisional Application Ser. No. 61/208,394, filed on Feb. 23, 2009 and U.S. Provisional Application Ser. No. 61/209,429, filed Mar. 6, 2009, each of which is incorporated by reference in its entirety, including all drawings and all parts of the specification (including sequence listing or amino acid/polynucleotide sequences).

### BACKGROUND OF THE INVENTION

Complementary oligonucleotide sequences are promising therapeutic agents and useful research tools in elucidating gene functions. However, prior art oligonucleotide molecules suffer from several problems that may impede their clinical development, and frequently make it difficult to achieve intended efficient inhibition of gene expression (including protein synthesis) using such compositions.

For example, classic siRNAs have limitations and drawbacks that may result in those agents being only moderately useful as human therapeutics. Specifically, classic siRNA is double-stranded. For each molecule, two strands need to be synthesized and paired up. Classic siRNA is made from naturally occurring ribonucleotides and is vulnerable to nucleases and spontaneous hydrolysis. The strands of classic siRNA are paired to each other except for an overhang of one strand at each end, and are about 19 to 23 nucleotides long. This configuration limits the variety and activity of the compound. For example, longer oligonucleotides can have higher binding activity to target RNA, which often correlates with higher activity. The overhangs of classic siRNA cause instability (because single strands are more nuclease resistant than double strands in most cases) and degradation, and may be the cause of the molecules “sticking” to each other or other nucleotides.

In addition, it is widely believed that double-stranded RNAs longer than 21-mer are cleaved by Dicer or Dicer-like RNAse III in mammalian cells, resulting in classic siRNA products. One strand of the Dicer-cleavage products is then loaded onto the RISC complex, and guides the loaded RISC complex to effect RNA interference (RNAi). However, since Dicer is not sequence specific, the Dicer-cleavage products of unmodified long dsRNA is a heterogeneous mixture of 21-mers, each may have different biological activity and/or pharmacological property. In addition, each 21-mer may have a distinct off-target effect (e.g., inhibiting the function of an unintended target due to, for example, spurious sequence homology between the Dicer cleavage product and target mRNAs). In other words, the active drug (e.g., the 21-mers) may be multiple species with relatively unpredictable target specificities, biological activities and/or pharmacological properties. Also, Dicer product is shorter than the parent, which leads to a lower affinity guide strand.

Other problems include the susceptibility of the siRNAs to non-specific nuclease degradation when applied to biological systems. Therefore, it would be of great benefit to improve upon the prior art oligonucleotides by designing improved oligonucleotides that either are free of or have reduced degree of the above-mentioned problems.

### SUMMARY OF THE INVENTION

The present invention is directed to compositions and methods pertaining to unique polynucleotide constructs, such as hairpin nucleic acids, for use in gene silencing. Accordingly, the present invention provides compositions and methods for increasing the efficiency of RNA interference.

Thus one aspect of the invention provides a polynucleotide construct comprising two identical single-stranded polynucleotides, each of the single-stranded polynucleotide comprising a 5′-stem sequence having a 5′-end, a 3′-stem sequence having a 3′-end, and a linker sequence linking the 5′-stem sequence and the 3′-stem sequence, wherein: (1) the 5′-stem sequence of a first single-stranded polynucleotide hybridize with the 3′-stem sequence of a second single-stranded polynucleotide to form a first double-stranded stem region; (2) the 5′-stem sequence of the second single-stranded polynucleotide hybridize with the 3′-stem sequence of the first single-stranded polynucleotide to form a second double-stranded stem region; and, (3) the linker sequences of the first and the second single-stranded polynucleotides form a loop or bulge connecting the first and the second double-stranded stem regions, wherein the 5′-stem sequence and at least a portion of the linker sequence form a guide sequence complementary to a transcript of a target gene, wherein the polynucleotide construct mediates sequence-dependent gene silencing of expression of the target gene.

In certain embodiments, the 5′-stem sequence, the loop, and at least a portion of the 3′-stem sequence collectively form the guide sequence complementary to the transcript of the target gene.

In certain embodiments, the guide sequence is about 15-21 nucleotides in length, or about 17-21 nucleotides in length, or about 19-21 nucleotides in length, or about 17-18 nucleotides in length.

In certain embodiments, each of the single-stranded polynucleotides is about 15-49 nucleotides in length, about 33-35 nucleotide in length, or about 25-27 nucleotides in length, or about 25-32 nucleotides in length.

In certain embodiments, the polynucleotide construct is capable of associating with a RISC complex.

In certain embodiments, the polynucleotide construct is not a substrate for Dicer.

In certain embodiments, each of the first and second double-stranded stem regions is less than 21 bp in length.

In certain embodiments, each of the first and second double-stranded stem regions is less than about 20 base pairs in length, or is about 5-15 base pairs in length, or about 11-14 base pairs in length.

In certain embodiments, each of the double-stranded regions is at least 8, 9, 10, or 11 base pairs in length, preferably at least 12 base pairs in length.

In certain embodiments, the linker sequence is 1, 2, 3, 4, 5, 6, 7, 8, 9, 10, 11, 12, 13, or 14 nucleotides in length.

In a preferred embodiment of the invention, the linker sequences are not complementary and do not hybridize to each other.

In a preferred embodiment of the invention, the linker sequences are not complementary and do not hybridize to each other.

In a preferred embodiment of the invention, the linker sequences are not complementary and do not hybridize to each other.

In certain embodiments, the subject polynucleotide construct comprises an overhang on the 3′-end and/or an overhang on the 5′-end.

In certain embodiments, the target gene transcript is a messenger RNA (mRNA).

In certain embodiments, each of the two identical single-stranded polynucleotides is an RNA.

In certain embodiments, at least 8, 10, or 12 nucleotides from the 5′-end of the polynucleotide are 100% complementary to the target gene transcript.

In certain embodiments, at least one nucleotide is modified to improve resistance to nucleases, serum stability, target specificity, blood system circulation, tissue distribution, tissue penetration, cellular uptake, potency, and/or cell-permeability of the polynucleotide.

In certain embodiments, the modified nucleotides are modified on the sugar moiety, the base, and/or the phosphodiester linkage.

In certain embodiments, the modification is a phosphate analog.

In certain embodiments, the modification is a phosphorothioate linkage.

In certain embodiments, the phosphorothioate linkage is limited to one or more nucleotides within the loop, a 5′-overhang, and/or a 3′-overhang.

In certain embodiments, the phosphorothioate linkage is limited to one or more nucleotides within the loop, and 1, 2, 3, 4, 5, or 6 more nucleotide(s) of the guide sequence within the double-stranded stem region just 5′ to the loop.

In certain embodiments, the total number of nucleotides having the phosphorothioate linkage is about 12-14.

In certain embodiments, all nucleotides having the phosphorothioate linkage are not contiguous.

In certain embodiments, the modification is at position 2 from the 5′-end of the single-stranded polynucleotide.

In certain embodiments, the modification is a 2′-O-alkyl or 2′-halo group.

In certain embodiments, the modification comprise 2′-O-methyl modification at alternative nucleotides, starting from either the first or the second nucleotide from the 5′-end.

In certain embodiments, the modification comprise 2′-O-methyl modification of one or more randomly selected pyrimidine nucleotides (C or U).

In certain embodiments, the modification comprises 2′-O-methyl modification of one or more nucleotides within the loop.

In certain embodiments, the modification is either limited to one or more nucleotides within the loop, or additionally including 1, 2, 3, 4, 5, or 6 more nucleotide(s) of the guide sequence within the double-stranded stem region just 5′ to the loop.

In certain embodiments, the modification comprise 2′-O-methyl modification, wherein no more than 4 consecutive nucleotides are modified.

In certain embodiments, all nucleotides in the 3′-end stem region are modified.

In certain embodiments, all nucleotides 3′ to the loop are modified.

In certain embodiments, the modification comprise hydrophobic modification to one or more bases.

In certain embodiments, the one or more bases are C or G.

In certain embodiments, the hydrophobic modification comprise an isobutyl group.

In certain embodiments, the 3′-stem sequence is 100% complementary to the 5′-stem sequence in each double-stranded stem regions.

In certain embodiments, the 3′-stem sequence is less than 100% complementary to the 5′-stem sequence.

In certain embodiments, the 5′- or 3′-stem sequence comprises one or more universal base-pairing nucleotides.

In certain embodiments, the target gene is present in a cell.

In certain embodiments, the target gene is an endogenous gene.

In certain embodiments, the target gene is a pathogen-derived exogenous gene.

In certain embodiments, the cell is of eukaryotic origin.

In certain embodiments, the cell is from a mammal, nematode, or insect.

In certain embodiments, the sequence-dependent gene silencing is mediated by an miRNA mechanism.

Another aspect of the invention provides a composition comprising any of the subject polynucleotide constructs.

In certain embodiments, the composition further comprises the single-stranded polynucleotides having a different structure from that of the polynucleotide construct.

In certain embodiments, at least about 50%, 60%, 70%, 80%, 90% or more (w/w) of the single-stranded polynucleotides are present in the polynucleotide construct.

Another aspect of the invention provides a pharmaceutical composition comprising any of the subject composition, and a pharmaceutically acceptable salt, diluent, excipient, or carrier.

Another aspect of the invention provides a method of treating a patient for a disease characterized by overexpression of a target gene, comprising administering to the patient a subject polynucleotide construct, wherein the polynucleotide construct mediates guide sequence-dependent reduction in expression of the target gene.

Another aspect of the invention provides a method of inhibiting expression of a target gene with a subject polynucleotide construct, wherein the polynucleotide construct mediates guide sequence-dependent reduction in expression of the target gene.

Another aspect of the invention provides a polynucleotide construct comprising a first single-stranded polynucleotide and a second single-stranded polynucleotide, each comprising a 5′-stem sequence having a 5′-end, a 3′-stem sequence having a 3′-end, and a linker sequence linking the 5′-stem sequence and the 3′-stem sequence, wherein: (1) the 5′-stem sequence of the first single-stranded polynucleotide hybridize with the 3′-stem sequence of the second single-stranded polynucleotide to form a first double-stranded stem region; (2) the 5′-stem sequence of the second single-stranded polynucleotide hybridize with the 3′-stem sequence of the first single-stranded polynucleotide to form a second double-stranded stem region; and, (3) the linker sequences of the first and the second single-stranded polynucleotides form a loop or bulge connecting said first and said second double-stranded stem regions, wherein the 5′-stem sequence and at least a portion of the linker sequence for said first single-stranded polynucleotide form a first guide sequence complementary to a transcript of a first target gene, and the 5′-stem sequence and at least a portion of the linker sequence for said second single-stranded polynucleotide form a second guide sequence complementary to a transcript of a second target gene, and, wherein said polynucleotide construct mediates sequence-dependent gene silencing of expression of said first and second target genes.

In certain embodiments, the first target gene and the second target gene are different genes. Such genes may be functionally related (such as different genes in the same biological pathway or synergistic pathways) or unrelated. This can be useful when, for example, two genes required for certain disease conditions (such as two oncogenes in cancer) can be simultaneously targeted by the same pharmaceutical composition.

In other embodiments, the first target gene and the second target gene are different regions of the same gene. This can be helpful to achieve synergistic inhibition of the same gene.

In another aspect, the invention is a single-stranded polynucleotide that forms a hairpin structure, which includes a double-stranded stem and a single-stranded loop, wherein said polynucleotide mediates sequence-dependent gene silencing of the target gene expression. The double-stranded stem comprises a 5′-stem sequence having a 5′-end, and a 3′-stem sequence having a 3′-end. In certain embodiments, the 5′-stem sequence and at least a portion of the loop form a guide sequence that is complementary to a transcript of a target gene, wherein the target gene transcript is a messenger RNA (mRNA). In another embodiment, the 5′-stem sequence, said loop, and at least a portion of the 3′-stem sequence collectively form the guide sequence complementary to a transcript of a target gene. In certain aspects, the single-stranded polynucleotide is an RNA.

In certain embodiments, at least 12 nucleotides beginning from the 5′-end of the single-stranded polynucleotide are 100% complementary to the target gene transcript. In another embodiment, the first 12 to about 15 nucleotides from the 5′-end of the polynucleotide are 100% complementary to the target gene transcript.

In another embodiment, at least one nucleotide of the hairpin structure is modified to improve its resistance to nucleases, serum stability, target specificity, tissue distribution, and/or cell permeability. In certain embodiments, said modification is in the 3′-stem sequence of the double-stranded stem. In one aspect, the modification is at position 2 from the 5′-end of the polynucleotide. Examples of such modification include a 2′O-alkyl or 2′-halo group, or a phosphate analog. In addition to the sugar moiety, the base and/or the phosphodiester linkage of the polynucleotide may also be modified.

In certain embodiments, the length of the polynucleotide is about 15-49 nucleotides, or about 25-26 nucleotides. In certain embodiments, the double-stranded stem of the polynucleotide is less than 21 base pairs in length. In further embodiments, the double-stranded stem of the polynucleotide is less than about 20 base pairs in length, or is about 5-15 base pairs in length, or about 10 base pairs in length. In other embodiments, the length of the guide sequence is about 15-40 nucleotides, about 15-21 nucleotides, or about 19-21 nucleotides. Further, the single-stranded loop of the polynucleotide may be about 4, 5, 6, 7, 8, 9, 10, or 11 nucleotides in length. Additionally, the polynucleotide may further comprise an overhang on the 3′-end and/or an overhang on the 5′-end. Each overhang may have one or more nucleotides, which may comprise DNA and/or RNA, or modified analogs thereof.

In another embodiment, the 3′-end of the polynucleotide contains universal base-pairing nucleotides. In certain other embodiments, the 3′-stem sequence of the double-stranded stem of the polynucleotide contains one or more universal base-pairing nucleotides. In other embodiments, the 3′-stem sequence is 100% complementary, or less than 100% complementary to the 5′-stem sequence.

In certain embodiments, the foregoing single-stranded polynucleotide is capable of associating with a RISC complex. In other embodiments, the polynucleotide is not a substrate for Dicer or Dicer-like RNase III enzymes.

In certain embodiments, the target gene is an endogenous gene, or a pathogen-derived exogenous gene. The target gene may be present in a eukaryotic cell of mammalian, nematode, or insect origin, for example.

In certain embodiments, the subject single-stranded polynucleotide mediates sequence-dependent gene silencing by an miRNA mechanism. In other embodiments, the subject single-stranded polynucleotide mediates sequence-dependent gene silencing by an siRNA mechanism.

Another aspect of the invention relates to a method of treating a patient for a disease characterized by overexpression of a target gene, comprising administering to the patient any of the foregoing single-stranded polynucleotides, wherein the single-stranded polynucleotide mediates guide sequence-dependent reduction in expression of the target gene.

A further aspect of the invention relates to a method of inhibiting expression of a target gene using the foregoing single-stranded polynucleotide, wherein the single-stranded polynucleotide mediates guide strand-dependent reduction in expression of the target gene.

The composition may optionally include a pharmaceutical carrier and/or be formulated in a delivery device. In some embodiments the delivery device is selected from the group consisting of cationic lipids, cell permeating proteins, and sustained release devices. In one embodiment the sustained release device is a biodegradable polymer or a microparticle.

In some embodiments the 3′ terminal 10 nucleotides of the first single-stranded polynucleotide include at least two phosphate modifications, 4-14 phosphate modifications or the 3′ terminal 6 nucleotides of the first single-stranded polynucleotide all include phosphate modifications. In other embodiments the nucleotide in position one of the first single-stranded polynucleotide has a 5′ Phosphate modification. In yet other embodiments the phosphate modifications are phosphorothioate modifications. In some embodiments at least two nucleotides on the second single-stranded polynucleotide are phosphorothioate modified.

The first single-stranded polynucleotide may include at least one 2′-O-methyl modification or 2′-fluoro modification. In some embodiments the nucleotide in position one of the first single-stranded polynucleotide has a 2′-O-methyl modification. In other embodiments at least one C or U nucleotide in positions 2-10 of the first single-stranded polynucleotide has a 2′-fluoro modification. In yet other embodiments at least one C or U nucleotide in positions 11-18 of the first single-stranded polynucleotide has a 2′-O-methyl modification.

The first guide sequence, in some embodiments comprises the 5′ stem, the loop and at least one nucleotide of the 3′ stem in the first single-stranded polynucleotide. In other embodiments the first guide sequence comprises the 5′ stem, the loop and at two nucleotides of the 3′ stem in the first single-stranded polynucleotide. In yet other embodiments the second guide sequence comprises the 5′ stem, the loop and at least one nucleotide of the 3′ stem in the second single-stranded polynucleotide. The second guide sequence in other embodiments comprises the 5′ stem, the loop and at two nucleotides of the 3′ stem in the second single-stranded polynucleotide.

In another aspect, the invention is a single-stranded polynucleotide of less than 35 nucleotides in length that forms a hairpin structure, said hairpin includes a double-stranded stern and a single-stranded loop, said double-stranded stem having a 5′-stem sequence having a 5′-end, and a 3′-stem sequence having a 3′-end; and said 5′-stem sequence and at least a portion of said loop form a guide sequence complementary to a transcript of a target gene, wherein said polynucleotide mediates sequence-dependent gene silencing of expression of said target gene. I some embodiments the 5′-stem sequence, said loop, and at least a portion of said 3′-stem sequence collectively form the guide sequence complementary to said transcript of said target gene. In other embodiments the target gene transcript is a messenger RNA (mRNA). The single-stranded polynucleotide may be an RNA. In other embodiments the single-stranded polynucleotide is 25-26 nucleotides in length.

A method of treating a patient for a disease characterized by overexpression of a target gene is provided according to other aspects of the invention. The method involves administering to the patient a therapeutically effective amount of a polynucleotide construct described herein wherein the polynucleotide construct mediates guide sequence-dependent reduction in expression of the target gene. In some embodiments the therapeutically effective amount is a picomolar concentration.

A composition of a polynucleotide for use in the treatment of a patient for a disease characterized by overexpression of a target gene is also provided as an aspect of the invention.

Use of a polynucleotide for treating a patient for a disease characterized by overexpression of a target gene is also provided as an aspect of the invention.

A method for manufacturing a medicament of a polynucleotide for treating a patient for a disease characterized by overexpression of a target gene is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

It is contemplated that any of the embodiments described herein, including those in the examples, and those described under different aspects of the invention, can be combined with any other embodiments whenever applicable.

### BRIEF DESCRIPTION OF THE DRAWINGS

FIGS. 1A-1C show certain possible foldings of the subject polynucleotide constructs. FIG. 1A shows one possible folding (a hairpin structure) of a single-stranded polynucleotide used to make the subject polynucleotide constructs. Nucleotides 2-8 in FIG. 1A represent the seed region nucleotides. The first 15 nucleotides from the 5′-end base-pair perfectly to the target mRNA, while the rest may also anneal to provide efficient binding to the target polynucleotide. The duplex region of the construct may be recognized and loaded onto the RISC complex, resulting in the incorporation of the intact single-stranded polynucleotide into the RISC complex. “N” in FIG. 1A stands for canonical DNA or RNA nucleotide, while “N*” designates certain possible positions (non-limiting) for incorporating universal base-pairing nucleotides. The length of the stem and the size of the loop shown are arbitrary and can be varied in other embodiments. The single-stranded polynucleotide in FIG. 1A may also fold into a construct shown in FIG. 1B, a double-stranded construct formed by two identical single-stranded polynucleotides. The specific double-stranded construct in FIG. 1B has two 13-bp stem regions (one on each end of the construct) and a loop or bulge with 6 bases on each strand. The first 19 nucleotides in this single-stranded polynucleotide is the guide sequence that mediates inhibition of a target gene via RNAi mechanism. The guide sequence comprises the 13-nucleotide 5′-end stem sequence that forms one duplex region, and the 6 nucleotides in the loop region. Such double-stranded constructs are sometimes referred to as “solo-rxRNA” herein. Shown in FIG. 1C is an exemplary hairpin structure having a 14-bp duplex region and a 4-nucleotide loop region. Two such single-stranded polynucleotides can form a similar construct shown in FIG. 1B. As shown herein, there is no optional 5′- or 3′-overhang sequences in the constructs of FIGS. 1B and 1C.

FIGS. 2A-2E show several exemplary single-stranded polynucleotides designed to target the PPIB (peptidylprolyl isomerase B) sequence. Similar to FIG. 1B, two strands of sequences in FIGS. 2A through 2E may form stem regions having 14, 13, 12, 11, or 10 nucleotides, respectively, at both ends in the subject double-stranded constructs. The first 16-19 nucleotides from the 5′-end base-pair perfectly to the target mRNA, while the rest may also anneal to provide efficient binding to the target polynucleotide. The duplex regions may be recognized and loaded onto the RISC complex, resulting in the incorporation of the intact single-stranded polynucleotide into the RISC complex. Regular-boxed nucleotides in FIGS. 2A-2E likely will form the loop region in the mini hairpin conformation, but one or more of such nucleotides may become part of the duplex stem regions in the so-rxRNA conformation; bold-boxed nucleotides indicate positions that may be varied to include either a mismatch or a universal base. The sequences for the single-stranded polynucleotides shown in FIGS. 2A-2E are as follows, with the PPIB sequence double-underlined and italicized:

A (10832): 5′UUUUUGGAACAGUCUUUCCAGACUGUUCCAAAAA3′ (SEQ ID NO: 1)

B (10833): 5′UUUUUGGAACAGUCUUUCCACUGUUCCAAAAA3′ (SEQ ID NO: 2)

C (10834): 5′UUUUUGGAACAGUCUUUCCUGUUCCAAAAA3′ (SEQ ID NO: 3)

D (10835): 5′UUUUUGGAACAGUCUUUXXUUCCAAAAA3′ (SEQ ID NO: 4)

E (10836): 5′UUUUUGGAACAGUCUUXXXCCAAAAA3′ (SEQ ID NO: 5)

FIG. 3 illustrates some exemplary PPIB and MAP4K4-specific sequences that can be used to form the subject double-stranded polynucleotide constructs, some of which are also shown in a table in Example 4. For simplicity, only the hairpin structures are shown. However, these strands can form the solo-rxRNA duplexes as described in FIG. 1B. Certain hairpin structures depict the terminal base pair (such as A-U) as being “open” (i.e., not base-paired), suggesting that such terminal base pair may, under certain circumstances, be open at least temporarily. In addition, rxRNA constructs 10460 and 11546, previously shown to be effective in reducing PPIB and MAP4K4 expression, respectively, were used as positive controls. These controls (and other references to “rxRNA” as used herein) do not form solo-rxRNA, and contain modified bases on certain nucleotides. For example, rxRNA 10460 has 2′-O-methyl modification on 4 of the outer-most positions at both ends of the sequences; rxRNA 11546 has 2′-O-methyl modification on the twelve (12) 5′-end nucleotides and the ten (10) 3′-end nucleotides.

FIG. 4 shows a single-stranded polynucleotide having a 2-bp overhang at the 3′ end. This represents an exemplary negative control. As shown, the PPIB sequence is positioned at the 3′ end of the polynucleotide and is therefore not expected to be as effective in reducing PPIB gene expression as compared to the constructs of the present invention. Similar to FIG. 2 above, unbolded boxed nucleotides will likely form the loop in the mini hairpin structure, but additional base-pairing may occur in the solo-rxRNA conformation. The PPIB sequence within the polynucleotide is double-underlined and italicized. Negative control (10837):

(SEQ ID NO: 6)
5′AAAAACCUUGUCAGAAAGGUUCAAGAGACCUUUCUGACAAGGUUU
UUUU3′.

FIG. 5 indicates that two exemplary polynucleotide constructs of the present invention, 10833 and 10834, can inhibit PPIB expression in HEK293 cells at a range of concentrations. As used herein, “13-nt stem” (or other similar designations) means the construct has two 13-nucleotide stem regions when it is in the solo-rxRNA conformation. The relative expression of remaining PPIB after transfection is compared to both negative and positive control constructs, 10837 and 10460, respectively. UTC indicates untransfected control. The specific sequence of each construct is detailed in the Examples below.

FIG. 6 shows the relative expression of the PPIB target gene after transfection with the subject polynucleotide constructs (10833 and 10834) that antagonize PPIB expression. Additionally, two dsRNAs were included as positive controls with known efficacy at silencing PPIB expression (10460 and 10167.2).

FIG. 7 shows the percentage expression of the PPIB target gene 48 hours after transfection with the subject polynucleotide constructs (10833 and 10834) that antagonize PPIB expression. Additionally, two dsRNAs were included as positive controls with known efficacy at silencing PPIB expression (10460 and 10167.2). The experiment was conducted over a range of concentrations for the different tested constructs. The data obtained can be used to determine EC₅₀ values for effective constructs.

FIG. 8 shows dose-response curves for PPIB expression using a 13-bp-stem and a 12-bp-stem polynucleotide construct, in comparison with those of the more traditional longer dsRNA constructs. The plot may be used to determine EC₅₀ values for effective constructs.

FIG. 9 shows the results of an experiment conducted to determine the minimal length of stem region in the effective polynucleotide constructs that antagonize PPIB expression. The stem can be as short as 12 nt in this experiment. Constructs with 11 and 10 nucleotide stems contain at least one inosine modification.

FIG. 10 shows that gene silencing mediated by the subject polynucleotide constructs is specific to construct structures. The negative control sequences were short blunt-ended dsRNA of the specified length.

FIG. 11 shows that gene silencing mediated by the subject polynucleotide constructs is sequence specific. The dsRNA construct 10460 is more potent than the dsRNA construct 10463. The subject polynucleotide constructs designed based on the 10460 sequence (11975 and 11976) are more potent that those designed based on the 10463 sequence (12003 and 12004). This demonstrates that the gene silencing activity of the subject polynucleotide constructs are sequence specific.

FIG. 12 shows several single-stranded polynucleotides that can be used to form the subject constructs and their corresponding RNAi activity. Five PPIB sequences were examined against three other control constructs. For simplicity, only hairpin structures are depicted for each sequence in this figure (terminal A-U pair shown as “open” to represent possible temporary non-base-pairing state). However, these sequences can and likely do form the solo-rxRNA constructs as described in FIG. 1B (see below).

FIG. 13 shows the minimal length of stem regions in the effective polynucleotide constructs that antagonize MAP4K4 expression. The control sequences were blunt-ended dsRNA of the specified length. As used herein (also see above), “stem” denotes the number of nucleotides in the stem regions of the solo-rxRNA; “dsRNA” indicates that the corresponding construct is a double-stranded structure containing only one stem region of the solo-rxRNA.

FIG. 14 illustrates some exemplary sequences tested in FIG. 13 (shown in the form of short hairpin structures for simplicity). These sequences can also form the solo-rxRNA structures. Their relative activity is also demonstrated and compared to positive control rxRNA construct 11546.

FIG. 15 shows the results of experiments designed to determine the minimal length of the stem region in the effective polynucleotide constructs that antagonize SOD1 expression.

FIGS. 16A (for SOD1-targeting sequences) and 16B (for MAP4K4-targeting sequences) show that dimer (solo-rxRNA) and monomer (hairpin) run as distinct bands on electrophoresis gel. Constructs with different stem lengths are shown. Lane 1 is molecular weight marker (MWM). A dimer formation is faintly visible in lane 7 of FIG. 16A.

FIG. 17 shows the relative activities of constructs that antagonize SOD1 expression in view of varying stem region lengths. For simplicity, only hairpin structures are depicted for each sequence in this figure. However, these sequences can also form the double-stranded solo-rxRNA constructs as described in FIG. 1B.

FIG. 18 shows several tested polynucleotide constructs with different stem lengths, for target genes PPIB, MAP4K4, and SOD1. Only the hairpin structures are shown. However, two of the single-stranded polynucleotides can form the subject double-stranded polynucleotide constructs.

FIG. 19 shows several additional tested polynucleotide constructs with different stem/loop lengths, for target genes MAP4K4 and SOD1.

FIG. 20 shows the common structure of about fifteen 13-bp-stem sequences used for comparing activities with 25-mer dsRNA constructs. Only the hairpin structures are shown for the single-stranded polynucleotides. However, two of each of the single-stranded polynucleotides can form the subject double-stranded polynucleotide constructs with a central loop/bulge. “rxRNA” refers to (25 bp) blunt-ended dsRNA constructs with no central loop/bulge, but having 2′-OMe modifications at the 4 terminal 5′-end and 4 terminal 3′-end nucleotides on the sense strand.

FIG. 21 shows the structures of several controls used for target genes SOD1 and MAP4K4. Only the hairpin structure is shown for the single-stranded polynucleotide “parent.” However, two of the single-stranded polynucleotides can form the subject double-stranded polynucleotide constructs.

FIG. 22 demonstrates that the “stem-only” small duplex structure as shown in FIG. 21 does not have gene silencing activity. Here, solo-rxRNAs having 12-15 bp stem regions have comparable activity to a positive control 25 bp blunt rxRNA. However, each corresponding stem-only structures do not show silencing activity.

FIG. 23 shows several control constructs with progressive deletions of the sense sequence (3′-stem region sequence) in the duplex region. Only the hairpin structure is shown for the single-stranded polynucleotide that can form the subject double-stranded polynucleotide constructs.

FIG. 24 shows several constructs with different modification patterns. Only the hairpin structure is shown for the single-stranded polynucleotide that can form the subject double-stranded polynucleotide constructs.

FIG. 25 shows several constructs with different modification patterns. Only the hairpin structure is shown for the single-stranded polynucleotide that can form the subject double-stranded polynucleotide constructs.

FIG. 26 shows constructs with conjugated end groups (Dy547 or Cy3). Only the hairpin structure is shown for the single-stranded polynucleotide that can form the subject double-stranded polynucleotide constructs. Both ends of the single-stranded polynucleotide may be modified by identical or different end groups.

FIG. 27 shows several constructs with different phosphorothioate modification patterns. Only the hairpin structure is shown for the single-stranded polynucleotide that can form the subject double-stranded polynucleotide constructs.

FIG. 28 shows general correlation of activities between the subject solo-rxRNA polynucleotide constructs and their respective longer dsRNA (25-mer) constructs.

FIG. 29 demonstrates that a solo-rxRNA structure and a corresponding rxRNA duplex structure targeting the same seed region within the target gene MAP4K4 show comparable RNAi activity. This figure depicts only a hairpin structure for simplicity. However, this sequence can form the solo-rxRNA structure. EC₅₀ values are calculated based on the results obtained over a range of construct concentrations.

FIG. 30 also demonstrates that solo-rxRNA structures and the corresponding rxRNA duplex structures (denoted in figure as “duplex”) show comparable gene silencing activity when targeting the same seed region. Note that the potency ranking of the solo-rxRNA constructs is maintained compared to that of the rxRNA (e.g., a more effective rxRNA targeting a specific target sequence region over another is likely to give rise to a more effective solo-rxRNA if the seed region is preserved). This effect is shown over 3 difference concentrations, and by using 13- or 12-bp stem solo-rxRNA constructs.

FIG. 31, consistent with FIG. 28, also demonstrates that the position of the seed region within SOD1 is maintained when comparing the solo-rxRNA and the corresponding rxRNA duplex structures.

FIG. 32 shows inactivity of a short dsRNA and a nicked control with a 6-nt sense sequence 5′-overhang (cf. FIG. 21).

FIG. 33 contains examples of chemical modification patterns optimized for RISC entry, stability, and/or cellular uptake applied to the subject polynucleotide constructs. Optimal chemical modification pattern may contain majority or all of U's and C's in a guide region modified with 2′-F, and majority or all of U's and C's in a complementary region modified with 2′-OMe. Only the hairpin structure is shown for the single-stranded polynucleotide that can form the subject double-stranded polynucleotide constructs.

FIG. 34 shows exemplary constructs with the size of the loop being 6, 8, 10, or 12 nucleotides. The length of the modified single-stranded region might be important for uptake. The variation of the loop size might be due to the decrease in the duplex region size or increase in an overall length of the oligo. Note that loop size doubles in the corresponding solo-rxRNA constructs.

FIG. 35 shows certain structural variations in the subject polynucleotide constructs. For example, the construct may contain 1, 2, or 3 base pair overhang. Preferably, the 3′ overhang is a 2-nucleotide overhang. The overhang can be chemically modified (shown by “*”), such as phosphothioate modification. Only the hairpin structure is shown for the single-stranded polynucleotide that can form the subject double-stranded polynucleotide constructs.

FIG. 36 demonstrates that one or more stabilizing chemical modifications might be applied to the duplex region of subject polynucleotide constructs, and convert otherwise non-functional entities to functional ones. Only the hairpin structure is shown for the single-stranded polynucleotide that can form the subject double-stranded polynucleotide constructs.

FIG. 37 illustrates an exemplary derivative of the subject polynucleotide constructs (shown with 9 bp stem region), with the stem region connected by a flexible non-nucleotide linker, which connects the two duplex regions of the double-stranded polynucleotide constructs. Only the hairpin-like structure is shown for the single-stranded polynucleotide that can form the subject double-stranded polynucleotide constructs.

FIG. 38 shows several constructs with different types of conjugates attached to the end (e.g., the 3′-end) or the loop region. The constructs may additionally comprise other modifications to the sugar ring (2′-F or 2′-OMe, etc.) or the backbone (e.g., phosphorothioate). Only the hairpin structure is shown for the single-stranded polynucleotide that can form the subject double-stranded polynucleotide constructs.

FIG. 39 shows some exemplary predicted structures that result from two identical single-stranded polynucleotides described herein (over the alternative mini hairpin structures formed by one single-stranded polynucleotide).

FIG. 40 shows monomer or duplex formation of the various constructs illustrated in FIG. 39 as analyzed on gel electrophoresis. The constructs were prepared and reconstituted at 10 mM, in 3 M KCl, 30 mM HEPES buffer at pH 6.0. One set of samples were diluted directly in buffer and analyzed on gel. The other set of samples were first heated to 95° C. for about 2 minutes, and then dried down on a Speed-vac at ambient temperature. The dried-down samples were then reconstituted in buffer and analyzed on gel.

FIG. 41 illustrates the relative percentages of monomer and duplex formation by each construct illustrated in FIG. 39.

FIG. 42 similarly shows the relative dimer (solo-rxRNA duplex) to monomer (hairpin) formation as visualized by native gel for varying stem sizes of MAP4K4 and SOD1 sequences. The gene silencing activity for each construct is shown above.

FIG. 43 shows annealing conditions that preferentially give rise to monomer formation. This is demonstrated using constructs having 13 bp stem regions for both PPIB and MAP4K4. In the 11975 gel image, lane 1: 1 μM 11975; lane 2: 1 μM 11975 denatured at 90° C. for 5 minutes, and then immediately placed on ice; lane 3: 1 μM 11975 denatured at 90° C. for 5 minutes, cooled to room temperature for 30 minutes, and then placed on ice; lane 4: 1 μM 11975 denatured at 90° C. for 5 minutes, cooled to room temperature for 30 minutes, and then incubated at 37° C. for about 1 hour; lane 5: 1 μM 11975 denatured at 90° C. for 5 minutes, cooled to room temperature for 30 minutes, and then incubated at 37° C. for about 2 hours. In the 11990 gel image, lane 1: 1 μM 11990; lane 2: 1 μM 11990 denatured at 90° C. for 5 minutes, and then immediately placed on ice; lane 3: 1 μM 11990 denatured at 90° C. for 5 minutes, cooled to room temperature for 30 minutes, and then placed on ice; lane 4: 1 μM 11990 denatured at 90° C. for 5 minutes, cooled to room temperature for 30 minutes, and then incubated at 37° C. for about 1 hour; lane 5: 1 μM 11990 denatured at 90° C. for 5 minutes, cooled to room temperature for 30 minutes, and then incubated at 37° C. for about 2 hours.

FIG. 44 examines the potency of a MAP4K4 or PPIB-specific sequence as a solo-rxRNA duplex as compared to a re-annealed monomer form. As shown, the EC₅₀ of the solo-rxRNA structure is significantly lower than the construct that has been re-annealed to preferentially form the monomer (as shown in FIG. 43). In each case, a positive control using a corresponding rxRNA construct is also shown for comparison.

FIG. 45 represents a schematic diagram of an exemplary method by which the subject double-stranded polynucleotides may be formed. An alternative method is to anneal identical single-stranded polynucleotides under suitable conditions. “P” denotes phosphorylation. The underlined nucleotides represent unpaired bases (the central “loop/bulge” region) when the duplex is formed. The nucleotides in bold are modified nucleotides that have increased binding affinity to their base-pairing partner.

FIG. 46 illustrates a construct capable of targeting two different target genes (i.e., a dual targeting structure). In this particular example, one strand contains an SOD1 targeting sequence, while the other strand contains a PPIB targeting sequence. Each of the two guide sequences comprise one stem region sequence and a loop region sequence. Here, there is no optional 5′- or 3′-overhang sequences.

FIGS. 47A and 47B examines the efficacy of the dual targeting structure for its silencing activity against each the SOD1 or PPIB as compared to the corresponding solo-rxRNA and rxRNA. The dual-targeting construct is at least as effective compared to the PPIB solo-rxRNA, and more effective compared to the SOD1 solo-rxRNA.

FIG. 48A examines the relative dimer (solo-rxRNA) to monomer (hairpin as shown) formation for each construct shown for MAP4K4. For simplicity, only the hairpin structure is illustrated, however, each sequence is capable of forming the duplex solo-rxRNA structure, as demonstrated by native gel. The corresponding silencing activity for each sequence is shown in the upper graph. FIG. 48B illustrates a similar study for SOD1. A dimer band in the lane corresponding to the 9 bp stem is faintly visible.

FIG. 49 shows results from experiments for determining the effect of stem length and loop size on solo-rxRNA activity. In particular, constructs having 8, 10 or 12 bp stem regions, each with 3, 5, 7, 9, or 11 bp loops are compared within each group. The corresponding sequence and structure of the tested constructs are illustrated beneath each data set.

FIG. 50 shows exemplary sequences and structures with their tendency for dimer formation (exemplified by large negative AG values) as shown by native gel. The corresponding silencing activity for each construct is demonstrated. All dimers have significant gene silencing activity. Some dimers have at least one loop region base-pairing, resulting in more than one smaller loops or bulges formed by the linker sequences.

FIG. 51 shows that the transfected solo-rxRNA constructs, as indicated, immunoprecipitate with Ago2, demonstrating that the subject polynucleotides are capable of being loaded into Ago2.

FIG. 52A demonstrates that all solo-rxRNA constructs against SOD1 shown to be active are not processed by Dicer, demonstrating that Dicer cleavage is not required for the subject polynucleotides to be loaded onto the RISC complex and be active RNAi constructs. Similarly, FIG. 52B demonstrates the same for MAP4K4 constructs.

FIGS. 53A and 53B demonstrate the stability of exemplary SOD1 and MAP4K4 solo-rxRNA constructs in 20% human serum. Construct 12060 has 2′-OMe modification on C and U bases, with the small core region, which spans 9-13 nt (counting 5′ end to 3′ end), unmodified. Construct 12061 has 2′-OMe modification on C and U bases, with the larger core region, which spans 9-21 nt (counting 5′ end to 3′ end), unmodified.

FIG. 54 shows exemplary sequences and structures having different stem and loop sizes. The corresponding silencing activity for each construct is shown.

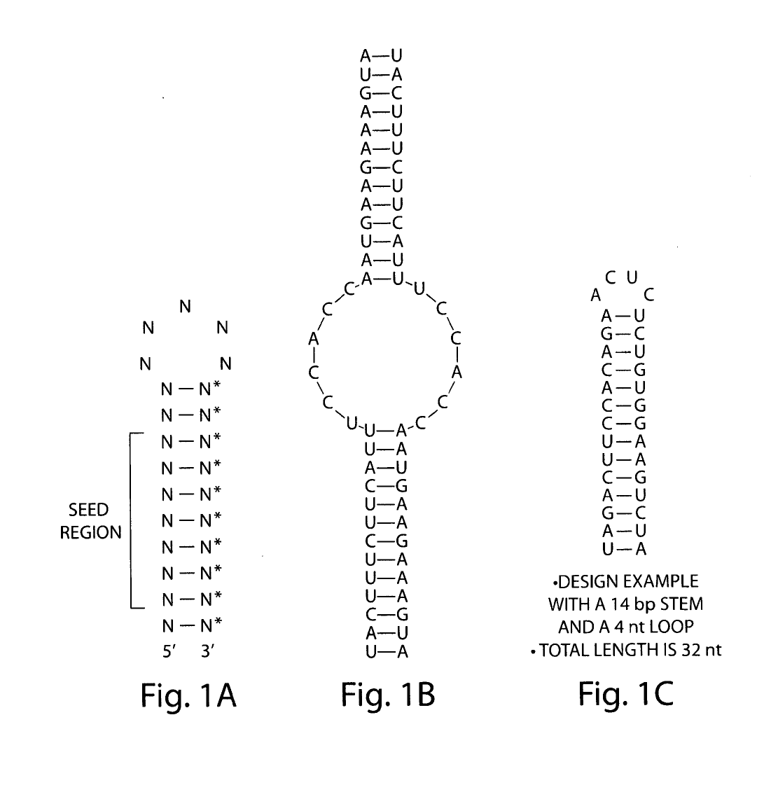

### DETAILED DESCRIPTION OF THE INVENTION

### 1. Overview

The invention is partly based on the discovery that a double-stranded structure with a central loop, formed from two single-stranded (partially palindromic) polynucleotides, does not require processing, and indeed is not processed by Dicer or other Dicer-like RNase III enzymes to participate in (RISC-mediated) RNA interference. A direct implication of Applicants' discovery is that the antisense (guide) strand of such a structure becomes the single species of active RNAi reagent, thus facilitating the development of RNAi reagents or therapeutics with higher target specificity, and better-defined biological activity and/or pharmacological property. Thus, in some embodiments a single oligonucleotide can be designed to form a duplex with identical strands. Furthermore, with the knowledge that such a construct can be engineered to resist Dicer cleavage, and the knowledge that the Dicer-resistant guide strand/sequence can be loaded onto the RISC complex at a defined location to create a single species of active RNAi reagent, one can engineer additional features or modifications into the guide sequence to improve the property of the RNAi reagent or therapeutics. Due to the symmetric nature of the construct, both single-stranded polynucleotides in the construct yield the same RISC complex.

Thus in one aspect, the invention provides a polynucleotide construct comprising two identical single-stranded polynucleotides, each of the single-stranded polynucleotide comprising a 5′-stem sequence having a 5′-end, a 3′-stem sequence having a 3′-end, and a linker sequence linking the 5′-stem sequence and the 3′-stem sequence, wherein: (1) the 5′-stem sequence of a first single-stranded polynucleotide hybridize with the 3′-stem sequence of a second single-stranded polynucleotide to form a first double-stranded stem region; (2) the 5′-stem sequence of the second single-stranded polynucleotide hybridize with the 3′-stem sequence of the first single-stranded polynucleotide to form a second double-stranded stem region; and, (3) the linker sequences of the first and the second single-stranded polynucleotides form a loop or bulge connecting the first and the second double-stranded stem regions, wherein the 5′-stem sequence and at least a portion of the linker sequence form a guide sequence complementary to a transcript (such as an mRNA or a non-coding RNA) of a target gene, wherein the polynucleotide construct mediates sequence-dependent gene silencing of expression of the target gene.

Another aspect of the invention provides a polynucleotide construct comprising a first single-stranded polynucleotide and a second single-stranded polynucleotide, each comprising a 5′-stem sequence having a 5′-end, a 3′-stem sequence having a 3′-end, and a linker sequence linking the 5′-stem sequence and the 3′-stem sequence, wherein: (1) the 5′-stem sequence of the first single-stranded polynucleotide hybridize with the 3′-stem sequence of the second single-stranded polynucleotide to form a first double-stranded stem region; (2) the 5′-stem sequence of the second single-stranded polynucleotide hybridize with the 3′-stem sequence of the first single-stranded polynucleotide to form a second double-stranded stem region; and, (3) the linker sequences of the first and the second single-stranded polynucleotides form a loop or bulge connecting said first and said second double-stranded stem regions, wherein the 5′-stem sequence and at least a portion of the linker sequence for said first single-stranded polynucleotide form a first guide sequence complementary to a transcript of a first target gene, and the 5′-stem sequence and at least a portion of the linker sequence for said second single-stranded polynucleotide form a second guide sequence complementary to a transcript of a second target gene, and, wherein said polynucleotide construct mediates sequence-dependent gene silencing of expression of said first and second target genes.

In certain embodiments, the first target gene and the second target gene are different genes. Such genes may be functionally related (such as different genes in the same biological pathway or synergistic pathways) or unrelated. This can be useful when, for example, two genes required for certain disease conditions (such as two oncogenes in cancer) can be simultaneously targeted by the same pharmaceutical composition.

In other embodiments, the first target gene and the second target gene are different regions of the same gene. This can be helpful to achieve synergistic inhibition of the same gene.

Preferably, the single-stranded polynucleotide is RNA, DNA, or hybrid thereof. “Hybrid” as used herein refers to a polynucleotide including both DNA and RNA nucleotides, although all DNA nucleotides need not be in a continuous stretch, and all RNA nucleotides need not be in a continuous stretch in the hybrid.

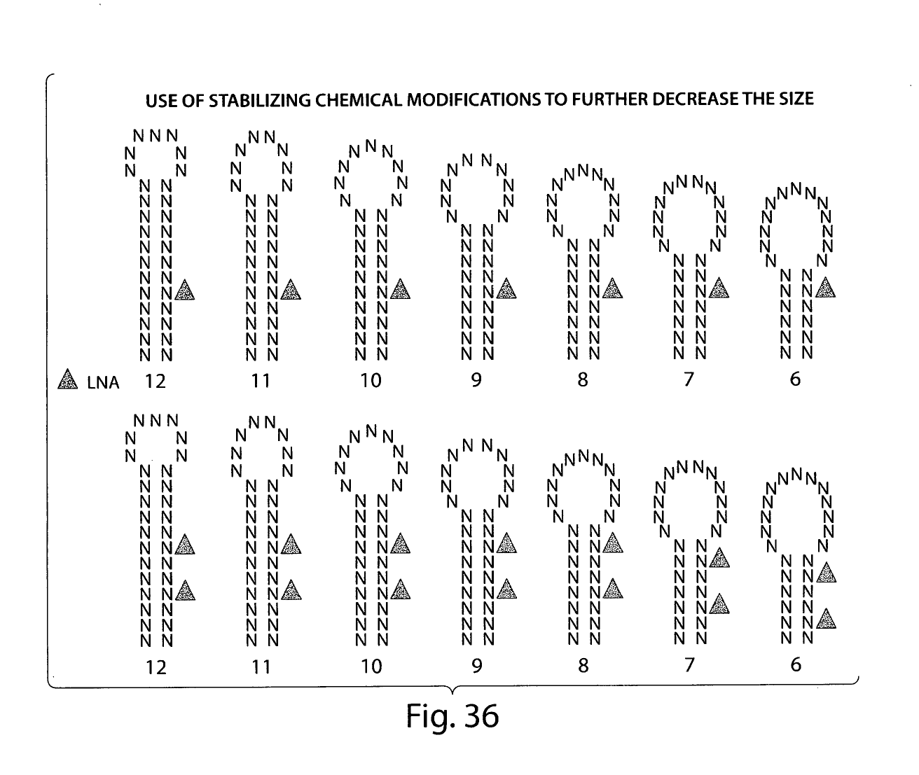

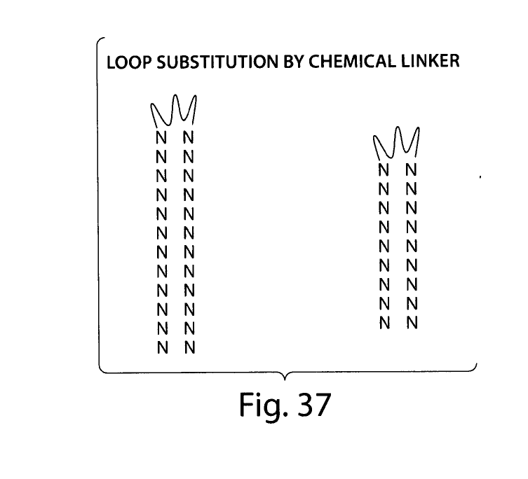

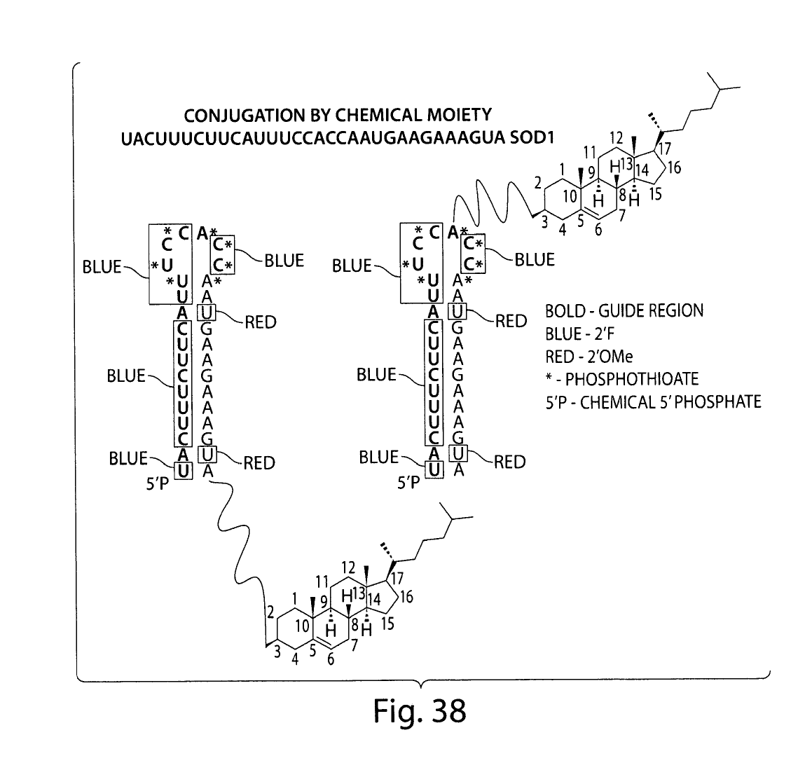

While not wishing to be bound by any particular theory, it is believed that the duplex/stem length limitation may be partially defined by thermodynamic stability in cellular environments. Thus a group of chemical modifications known to enhance thermodynamic stability of a duplex region may be used to alter stem length. A non-limiting example of these chemical modifications might be LNA (locked nucleic acid) or MGB (minor grove binder). There are other chemical modifications with similar properties in the art. FIG. 36 demonstrates that one or more stabilizing chemical modifications might be applied to the duplex region of the subject constructs and convert otherwise non-functional entities to functional ones. Preferably, the modification is in a non-guide sequence region.

Since chemically modified stem length can be as small as 6 base pairs, standard bioinformatics methods may be used to identify perfect or partially perfect inverted repeats (IR) regions and use them as target site for the subject constructs.

In certain embodiments, the 5′-stem sequence (including any 5′-end overhangs), the single-stranded loop, and at least a portion or all of the 3′-stem sequence form a guide strand/sequence that is complementary to the transcript of the target gene. Furthermore, the subject polynucleotide construct may be (1) resistant to cleavage by Dicer, (2) associates with RISC, and/or (c) inhibits expression of the target gene in a guide sequence-dependent manner.

In certain embodiments, the polynucleotide does not contain any overhangs. In other embodiments, 5′- and/or 3′-end overhangs of 1-6 nucleotides (preferably 1, 2, or 3 nucleotide overhang) may be present on one or both ends of the polynucleotide. The number and/or sequence of nucleotides overhang on one end of the polynucleotide may be the same or different from the other end of the polynucleotide. In certain embodiments, one or more of the overhang nucleotides may contain chemical modification(s), such as phosphothioate or 2′-OMe modification.

The constructs of the invention may have different lengths. In certain embodiments, the preferred lengths of the construct (including the two stem regions and the loop) are 12-49 nucleotides in length, 15-49 nucleotides in length, or 33-35 nucleotides in length, or about 25-32 nucleotides in length. In certain embodiments, the length of the construct is 25, 26, or 27 nucleotides in length. Preferably, each double-stranded stem region is about 8, 9, 10, 11, 12, or 13 bp in length. Shorter double-stranded region may be used without substantially reducing RNAi activity when certain modifications are included (such as LNA) to strengthen base-pairing in the short duplex region. Other lengths are also possible, so long as the double-stranded stem region does not exceed a maximum length causing it to be a Dicer substrate. In certain preferred aspects, the maximum length of each of the double-stranded stem region does not exceed 21 base pairs. In another aspect, the maximum length of the double-stranded stem region does not exceed 20, 19, about 5-15, or about 11-14 base pairs. Additionally, the double-stranded stem may be shorter than 10 base pairs without negatively affecting the RNAi capability of the construct. In other embodiments, the length of the single-stranded loop may be varied to allow for enhanced stability, and/or increased formation of the double-stranded polynucleotide construct (as opposed to single-stranded hairpin structures), for example. In certain embodiments, the loop region has 1, 2, 3, 4, 5, 6, 7, 8, 9, 10, 11, 12, 13, or 14 unpaired bases on each strand, preferably 2, 3, 4, 5, or 6 unpaired bases on each strand, more preferably 3 or 4 unpaired bases on each strand. In certain embodiments, there may be different numbers of unpaired bases on different strands.

In certain embodiments, the guide sequence within the construct is about 15-21 nucleotides in length, or about 17-21 nucleotides in length, or about 19-21 nucleotides in length, or about 17-18 nucleotides in length.

One advantage of the subject polynucleotide construct is the presence of single-stranded region (loop region). In some cases, the single-stranded (loop) region can be chemically modified to confer certain desired properties. For example, in some embodiments, the chemical modification may comprise phosphothioate. In some other embodiments, the chemical modification comprises 2′OME or 2′ Fluoro or 2′ deoxy. In yet other embodiments, the chemical modification is a combination of phosphorothioates with 2′ OMe and 2′ Fluoro. In other embodiments, the loop may be completely or partially replaced by a chemical linker that is flexible enough to allow the formation of equivalent duplex polynucleotides.

As used herein, the loop or bulge formed by the linker sequences need not be completely single-stranded throughout. Especially for relatively long linker sequences, such as those with about 5 or more nucleotides, one or more base-pairing may occur between the nucleotides on opposite single strands, or between the nucleotides on the same single strand. Therefore, the linker sequences may form one or more small loops or bulges. In other embodiments, the linker sequences do not form any base-pairings, small loops or bulges.

Modification of the subject polynucleotide constructs, if present, may also be present in nucleotides other than the loop nucleotides. According to this aspect of the invention, at least one nucleotide of the subject construct may be modified to improve its resistance to nucleases, serum stability, target specificity, blood system circulation, tissue distribution, tissue penetration, cellular uptake, potency, and/or cell-permeability of the polynucleotide. For example, certain guide strand modifications increase nuclease stability, and/or lower interferon induction, without significantly decreasing RNAi activity (or no decrease in RNAi activity at all). In certain embodiments, the modified polynucleotide constructs may have improved stability in serum and/or cerebral spinal fluid compared to an unmodified structures having the same sequence.

Therefore, in certain embodiments, the polynucleotide construct is unmodified. In other embodiments, at least one nucleotide in the construct is modified.

For example, in certain embodiments, the modification includes a 2′-H or 2′-modified ribose sugar at the 2^{nd} nucleotide from the 5′-end of the guide sequence. In certain embodiments, the guide strand (e.g., at least one of the two single-stranded polynucleotides) comprises a 2′-O-alkyl or 2′-halo group, such as a 2′-O-methyl modified nucleotide, at the 2^{nd} nucleotide on the 5′-end of the guide strand and, preferably, no other modified nucleotides. Polynucleotide constructs having such modification may have enhanced target specificity or reduced off-target silencing compared to a similar construct without the 2′-O-methyl modification at the position.

The “2^{nd} nucleotide” is defined as the second nucleotide from the 5′-end of the single-stranded polynucleotide.

As used herein, “2′-modified ribose sugar” includes those ribose sugars that do not have a 2′-OH group. “2′-modified ribose sugar” does not include 2′-deoxyribose (found in unmodified canonical DNA nucleotides), although one or more DNA nucleotides may be included in the subject constructs (e.g., a single deoxyribonucleotide, or more than one deoxyribonucleotide in a stretch or scattered in several parts of the subject constructs). For example, the 2′-modified ribose sugar may be 2′-O-alkyl nucleotides, 2′-deoxy-2′-fluoro nucleotides, 2′-deoxy nucleotides, or combination thereof.

In certain embodiments, the subject polynucleotide constructs with the above-referenced 5′-end modification exhibits significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the RNAi reagent or therapeutics.

As used herein, “off-target” gene silencing refers to unintended gene silencing due to, for example, spurious sequence homology between the antisense (guide) sequence and the unintended target mRNA sequence.

According to aspects of the invention, certain guide strand modifications further increase nuclease stability, and/or lower interferon induction, without significantly decreasing RNAi activity (or no decrease in RNAi activity at all). For example, the 5′-stem sequence may comprise a 2′-modified ribose sugar, such as 2′-O-methyl modified nucleotide, at the 2^{nd} nucleotide on the 5′-end of the polynucleotide and, preferably no other modified nucleotides. The hairpin structure having such modification may have enhanced target specificity or reduced off-target silencing compared to a similar construct without the 2′-O-methyl modification at said position.

In certain embodiments, the 2′-modified nucleotides are some or all of the pyrimidine nucleotides (e.g., C/U). Examples of 2′-O-alkyl nucleotides include 2′-O-methyl nucleotides, or 2′-O-allyl nucleotides.

In certain embodiments, the modification comprise 2′-O-methyl modification at alternative nucleotides, starting from either the first or the second nucleotide from the 5′-end.

In certain embodiments, the modification comprise 2′-O-methyl modification of one or more randomly selected pyrimidine nucleotides (C or U).

In certain embodiments, the modification comprises 2′-O-methyl modification of one or more nucleotides within the loop.

In certain embodiments, the modified nucleotides are modified on the sugar moiety, the base, and/or the phosphodiester linkage. The modification may be a phosphate analog, or a phosphorothioate linkage, which phosphorothioate linkage may be limited to one or more nucleotides within the loop, a 5′-overhang, and/or a 3′-overhang.

The phosphorothioate linkage may be limited to one or more nucleotides within the loop, and 1, 2, 3, 4, 5, or 6 more nucleotide(s) of the guide sequence within the double-stranded stem region just 5′ to the loop. In certain embodiments, the total number of nucleotides having the phosphorothioate linkage may be about 12-14. In certain embodiments, all nucleotides having the phosphorothioate linkage are not contiguous.

In certain embodiments, the modification comprise 2′-O-methyl modification, and no more than 4 consecutive nucleotides are modified.

In certain embodiments, all nucleotides in the 3′-end stem region are modified.

In certain embodiments, all nucleotides 3′ to the loop are modified.

In certain embodiments, the 5′- or 3′-stem sequence comprises one or more universal base-pairing nucleotides. Universal base-pairing nucleotides include extendable nucleotides that can be incorporated into a polynucleotide strand (either by chemical synthesis or by a polymerase), and pair with more than one pairing type of specific canonical nucleotide. In certain embodiments, the universal nucleotides pair with any specific nucleotide. In certain embodiments, the universal nucleotides pair with four pairing types of specific nucleotides or analogs thereof. In certain embodiments, the universal nucleotides pair with three pairing types of specific nucleotides or analogs thereof. In certain embodiments, the universal nucleotides pair\ with two pairing types of specific nucleotides or analogs thereof.

Universal base pairing nucleotides are known in the art, see, for example, Berger et al., “Universal bases for hybridization, replication and chain termination,” Nucleic Acids Research 28(15): 2911-2914, 2000; Loakes et al., “Survey and Summary: The applications of universal DNA base analogues,” Nucleic Acid Research 29(12): 2437-2447, 2001; Nichols et al., “A universal nucleoside for use at ambiguous sites in DNA primers,” Nature 369:492-493, 1994; Fasman, 1989, Practical Handbook of Biochemistry and Molecular Biology, pp. 385 394, CRC Press, Boca Raton, Fla., and the references cited therein; U.S. Pat. No. 7,169,557 (all incorporated herein by reference).

The universal nucleotide base may include an aromatic ring moiety, which may or may not contain nitrogen atoms. In certain embodiments, a universal base may be covalently attached to the C-1′ carbon of a pentose sugar to make a universal nucleotide. In certain embodiments, a universal nucleotide base does not hydrogen bond specifically with another nucleotide base. In certain embodiments, a universal nucleotide base may interact with adjacent nucleotide bases on the same nucleic acid strand by hydrophobic stacking. Exemplary universal nucleotides include, but are not limited to, inosine-based nucleotide, 2′-deoxy-7-azaindole-5′-triphosphate (d7AITP), 2′-deoxy-isocarbostyril-5′-triphosphate (dICSTP), 2′-deoxy-propynylisocarbostyril-5′-triphosphate (dPICSTP), 2′-deoxy-6-methyl-7-azaindole-5′-triphosphate (dM7AITP), 2′-deoxy-imidizopyridine-5′-triphosphate (d1 mPyTp), 2′-deoxy-pyrrollpyrizine-5′-triphosphate (dPPTP), 2′-deoxy-propynyl-7-azaindole-5′-triphosphate (dP7AITP), or 2′-deoxy-allenyl-7-azaindole-5′-triphosphate (dA7AITP), etc.

In certain embodiments, the modification comprise hydrophobic modification to one or more bases, such as C or G bases. In certain embodiments, the hydrophobic modification comprise an isobutyl group.

Certain combinations of specific 5′-stem sequence and 3′-stem sequence modifications may result in further unexpected advantages, as partly manifested by enhanced ability to inhibit target gene expression, enhanced serum stability, and/or increased target specificity, etc. Other potentially beneficial modifications are described in more detail in a separate section below.

To further increase the stability of the subject constructs in vivo, the 3′-end of the subject construct may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety.

In other aspects, the polynucleotide constructs of the present invention mediates sequence-dependent gene silencing by a microRNA mechanism. As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

microRNAs are involved in down-regulating target genes in critical pathways, such as development and cancer, in mice, worms and mammals. Gene silencing through a microRNA mechanism is achieved by specific yet imperfect base-pairing of the miRNA and its target messenger RNA (mRNA). Various mechanisms may be used in microRNA-mediated down-regulation of target mRNA expression.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNAs are expressed by endogenous genes in vivo and are processed from a hairpin or stem-loop precursor (pre-miRNA or pri-miRNAs) by Dicer or other RNAses. miRNAs can exist transiently in vivo as a double-stranded duplex but only one strand is taken up by the RISC complex to direct gene silencing.

Another pathway that uses small RNAs as sequence-specific regulators is the RNA interference (RNAi) pathway, which is an evolutionarily conserved response to the presence of double-stranded RNA (dsRNA) in the cell. The dsRNAs are cleaved into ˜20-base pair (bp) duplexes of small-interfering RNAs (siRNAs) by Dicer. These small RNAs get assembled into multiprotein effector complexes called RNA-induced silencing complexes (RISCs). The siRNAs then guide the cleavage of target mRNAs with perfect complementarity.

Some aspects of biogenesis, protein complexes, and function are shared between the siRNA pathway and the miRNA pathway. The subject polynucleotide constructs may mimic the dsRNA in the siRNA mechanism, or the microRNA in the miRNA mechanism. In certain embodiments, the subject polynucleotide construct is capable of associating with a RISC complex. In certain embodiments, the subject polynucleotide construct is not a substrate for Dicer.

In certain embodiments, the modified hairpin structure may have improved stability in serum and/or cerebral spinal fluid compared to an unmodified hairpin structures having the same sequence.

In other embodiments, at least the first 8, 10, 12 nucleotides from the 5′-end of the polynucleotide are 100% complementary to the target gene transcript. More preferably, at least the first 12 nucleotides from the 5′-end of the polynucleotide are 100% complementary to the target gene transcript. In certain preferred embodiments, about the first 12 to 15 nucleotides from the 5′-end of the polynucleotide are 100% complementary to the target gene transcript.

In certain embodiments, the 3′-stem sequence is less than 100% complementary to the 5′-stem sequence.

In certain embodiments, only nucleotides 2 to 17 of the guide sequence/strand is complementary to the target sequence. The sequence complementarity may be partial, preferably, the guide sequence can hybridize to the target sequence under the physiological condition of the cell or under high stringency condition.

In certain embodiments, the subject construct does not induce interferon response in primary cells, such as mammalian primary cells, including primary cells from human, mouse and other rodents, and other non-human mammals. In certain embodiments, the subject construct may also be used to inhibit expression of a target gene in an invertebrate organism.

To further increase the stability of the subject constructs in vivo, the 3′-end of the hairpin structure may be blocked by protective group(s). For example, protective groups such as inverted nucleotides, inverted abasic moieties, or amino-end modified nucleotides may be used. Inverted nucleotides may comprise an inverted deoxynucleotide. Inverted abasic moieties may comprise an inverted deoxyabasic moiety, such as a 3′,3′-linked or 5′,5′-linked deoxyabasic moiety.

The single-stranded polynucleotide constructs are capable of inhibiting the synthesis of any target RNA or protein encoded by the target gene(s). The invention includes methods to inhibit expression of a target gene either in a cell in vitro, or in vivo. As such, the polynucleotide constructs of the invention are useful for treating a patient with a disease characterized by the overexpression of a target gene.

The target gene can be endogenous or exogenous (e.g., introduced into a cell by a pathogen-derived exogenous gene, such as a virus gene, or introduced into a cell by using recombinant DNA technology) to a cell. Such methods may include introduction of RNA into a cell in an amount sufficient to inhibit expression of the target gene, where the RNA is a subject polynucleotide construct. By way of example, such an RNA molecule may have a guide strand that is complementary to the nucleotide sequence of the target gene, such that the composition inhibits expression of the target gene. As described in the foregoing embodiments, the guide strand may be formed by the 5′-stem sequence (including any 5′-end overhangs) and all or a portion of the single-stranded loop region. Alternatively, the guide strand may be formed by the 5′-stem sequence (including any 5′-end overhangs), the entire loop region, and all or a portion of the 3′-stem sequence.

The invention also relates to vectors expressing the subject polynucleotide constructs, and cells comprising such vectors or the subject polynucleotide constructs.

The cell may be of eukaryotic origin, and may be from a mammal, nematode, or insect. Mammalian cells include a mammalian cell in vivo or in culture, such as a human cell.

The invention further relates to compositions comprising the subject polynucleotide constructs. The composition may further comprise the single-stranded polynucleotides having a different structure from that of the polynucleotide construct. For example, within the composition, the single-stranded polynucleotides may form a single-stranded hairpin structure by self-annealing, instead of forming the subject double-stranded polynucleotide constructs. In certain preferred embodiments, at least about 50%, 60%, 70%, 80%, 90% or more (w/w) of the single-stranded polynucleotides are present in the polynucleotide construct. Or no more than 50%, 40%, 30%, 20%, or 10% of the single-stranded polynucleotides are present in their single-stranded form.

The invention further relates to a pharmaceutical composition comprising any of the subject compositions, and a pharmaceutically acceptable salt, diluent, excipient, or carrier.

Another aspect of the invention provides a method for inhibiting the expression of a target gene in a mammalian cell, comprising contacting the mammalian cell with any of the subject polynucleotide constructs.

The method may be carried out in vitro, ex vivo, or in vivo, in, for example, mammalian cells in culture, such as a human cell in culture.

The target cells (e.g., mammalian cell) may be contacted in the presence of a delivery reagent, such as a lipid (e.g., a cationic lipid) or a liposome.

Another aspect of the invention provides a method for inhibiting the expression of a target gene in a mammalian cell, comprising contacting the mammalian cell with a vector expressing the subject polynucleotide constructs.

Another aspect of the invention provides a method of inhibiting expression of a target gene with a subject polynucleotide construct, wherein the polynucleotide construct mediates guide sequence-dependent reduction in expression of the target gene.

More detailed aspects of the invention are described in the sections below.

### II. Polynucleotide Construct Structure

### Hairpin Characteristics

In a first embodiment, the hairpin structures of the present invention include a nucleic acid comprising a single-stranded RNA, such as a shRNA. The hairpin structure can include a double-stranded stem region formed from a 5′-stem sequence having a 5′-end (“5′-stem sequence”), and a 3′-stem sequence having a 3′-end (“3′-stem sequence”) that is complementary to the 5′-stem sequence. The hairpin structure can further include a single-stranded loop region.

The polynucleotide construct of the invention is formed by two identical or substantially identical single-stranded polynucleotides. Due to the partial palindromic nature of the sequence of the single-stranded polynucleotides, a double-stranded structure comprising two such single-stranded polynucleotides may form. Alternatively, the single-stranded polynucleotide may form a mini hairpin structure via intra-molecular base pairing. It is possible that, within a population of synthesized or purified such single-stranded polynucleotides, both structures may be present, but the ratio of the double-stranded structure over the single-stranded polynucleotide may vary, depending on a number of factors such as annealing conditions, structural features of the single-stranded polynucleotide (such as loop size or length, stem size or length, sequences of the loop/stem regions, G/C content, presence or absence of modifications, etc.), storage condition, the speed with which the two forms convert, etc.

Within the single-stranded polynucleotide, there is a 5′-stem sequence having a 5′-end (“5′-stem sequence”), and a 3′-stem sequence having a 3′-end (“3′-stem sequence”) that is complementary to the 5′-stem sequence. Between the stem sequences is a linker that may form a loop region in either form.

In a related embodiment, the single-stranded polynucleotide may be a DNA strand comprising one or more modified deoxyribonucleotides. In yet another related embodiment, the single-stranded polynucleotide may be an XNA strand, such as a peptide nucleic acid (PNA) strand or locked nucleic acid (LNA) strand. Further still, the single-stranded polynucleotide is a DNA/RNA hybrid.

Preferably the 5′-stem sequence and 3′-stem sequence are at least substantially complementary to each other, and more preferably about 100% complementary to each other. More preferably, the 5′-stem sequence and 3′-stem sequence are each 5 to 19 nucleotides, inclusive, in length. Alternatively, the 5′-stem sequence and 3′-stem sequence are each 10 to 19 nucleotides, inclusive, in length. In certain embodiments, the length of the stem region sequence may be more than 19 bp due to the presence of chemical modifications that prevent the stem region from being a Dicer substrate.

The 5′-stem sequence and 3′-stem sequence can be the same length, or differ in length by less than about 5 bases. The loop sequence is preferably about 2 to 15 nucleotides in length, and more preferably about 2, 3, or 4 nucleotides.

Overhangs, if any, may comprise between 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid. In the case of an overhang at the 5′-end of the polynucleotide, it is preferred that the modification(s) to the 5′-terminal nucleotide, if any, does not affect the RNAi capability of the hairpin construct. Such a modification can be, for example, a phosphorothioate.

As used herein, the term “double-stranded stem” includes one or more nucleic acid molecules comprising a region of the molecule in which at least a portion of the nucleomonomers are complementary and hydrogen bond to form a double-stranded region.

In certain embodiments, the 3′-stem sequence comprises one or more universal base-pairing nucleotides.

In certain embodiments, a double-stranded stems of the subject construct contains mismatches and/or loops or bulges, but is double-stranded over at least about 50% of the length of the double-stranded stem. In another embodiment, a double-stranded stern is double-stranded over at least about 60% of the length of the stem. In another embodiment, a double-stranded stem of the construct is double-stranded over at least about 70% of the length of the stem. In another embodiment, a double-stranded stem of the construct is double-stranded over at least about 80% of the length of the stem. In another embodiment, a double-stranded stem of the construct is double-stranded over at least about 90%-95% of the length of the double-stranded stem. In another embodiment, a double-stranded stem of the construct is double-stranded over at least about 96%-98% of the length of the stem. In certain embodiments, the double-stranded stem of the hairpin construct contains at least or up to 1, 2, 3, 4, 5, 6, 7, 8, 9, 10, 11, 12, 13, 14, or 15 mismatches. In certain embodiments, the mismatch may be at specific or non-specific positions, e.g., position 2 from the 5′ end, or position 1 on the 5′ end, etc.

### Modifications

The polynucleotide constructs of the invention may be modified at various locations, including the sugar moiety, the phosphodiester linkage, and/or the base.

Sugar moieties include natural, unmodified sugars, e.g., monosaccharide (such as pentose, e.g., ribose, deoxyribose), modified sugars and sugar analogs. In general, possible modifications of nucleomonomers, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One particularly useful group of modified nucleomonomers are 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified nucleomonomers may be used in combination with unmodified nucleomonomers. For example, an oligonucleotide of the invention may contain both methylated and unmethylated nucleomonomers.

Some exemplary modified nucleomonomers include sugar- or backbone-modified ribonucleotides. Modified ribonucleotides may contain a non-naturally occurring base (instead of a naturally occurring base), such as uridines or cytidines modified at the 5′-position, e.g., 5% (2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2% OH group replaced by a H, alxoxy (or OR), R or alkyl, halogen, SH, SR, amino (such as NH₂, NHR, NR₂), or CN group, wherein R is lower alkyl, alkenyl, or alkynyl.

Modified ribonucleotides may also have the phosphodiester group connecting to adjacent ribonucleotides replaced by a modified group, e.g., of phosphorothioate group. More generally, the various nucleotide modifications may be combined.

Although the guide sequence may be substantially identical to at least a portion of the target gene (or genes), at least with respect to the base pairing properties, the sequence need not be perfectly identical to be useful, e.g., to inhibit expression of a target gene's phenotype. Generally, higher homology can be used to compensate for the use of a shorter sequence. In some cases, the guide sequence generally will be substantially complementary to the target gene.

The use of 2′-O-methyl modified RNA may also be beneficial in circumstances in which it is desirable to minimize cellular stress responses. RNA having 2′-O-methyl nucleomonomers may not be recognized by cellular machinery that is thought to recognize unmodified RNA. The use of 2′-O-methylated or partially 2′-O-methylated RNA may avoid the interferon response to double-stranded nucleic acids, while maintaining target RNA inhibition. This may be useful, for example, for avoiding the interferon or other cellular stress responses, both in short RNAi (e.g., siRNA) sequences that induce the interferon response, and in longer RNAi sequences that may induce the interferon response.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary nucleomonomers can be found, e.g., in U.S. Pat. No. 5,849,902, incorporated by reference herein.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻ (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂—O—(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In a preferred embodiment, the nucleomonomers of an oligonucleotide of the invention are RNA nucleotides. In another preferred embodiment, the nucleomonomers of an oligonucleotide of the invention are modified RNA nucleotides. Thus, the oligonucleotides contain modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In certain embodiments, oligonucleotides of the invention comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

Another type of conjugates that can be attached to the end (3′ or 5′ end), the loop region, or any other parts of the subject construct might include a sterol, sterol type molecule, peptide, small molecule, protein, etc. In some embodiments, a subject construct may contain more than one conjugates (same or different chemical nature). In some embodiments, the conjugate is cholesterol.

Another way to increase target gene specificity, or to reduce off-target silencing effect, is to introduce a 2′-modification (such as the 2′-O methyl modification) at a position corresponding to the second 5′-end nucleotide of the guide sequence. This allows the positioning of this 2′-modification in the Dicer-resistant structure, thus enabling one to design better RNAi constructs with less or no off-target silencing.

In one embodiment, a subject polynucleotide construct can comprise one nucleic acid portion which is DNA and one nucleic acid portion which is RNA. The guide sequence can be “chimeric oligonucleotides” which comprise an RNA-like and a DNA-like region.

The language “RNase H activating region” includes a region of an oligonucleotide, e.g., a chimeric oligonucleotide, that is capable of recruiting RNase H to cleave the target RNA strand to which the oligonucleotide binds. Typically, the RNase activating region contains a minimal core (of at least about 3-5, typically between about 3-12, more typically, between about 5-12, and more preferably between about 5-10 contiguous nucleomonomers) of DNA or DNA-like nucleomonomers. (See, e.g., U.S. Pat. No. 5,849,902). Preferably, the RNase H activating region comprises about nine contiguous deoxyribose containing nucleomonomers.

The language “non-activating region” includes a region of an antisense sequence, e.g., a chimeric oligonucleotide, that does not recruit or activate RNase H. Preferably, a non-activating region does not comprise phosphorothioate DNA. The oligonucleotides of the invention comprise at least one non-activating region. In one embodiment, the non-activating region can be stabilized against nucleases or can provide specificity for the target by being complementary to the target and forming hydrogen bonds with the target nucleic acid molecule, which is to be bound by the oligonucleotide.

In one embodiment, at least a portion of the contiguous polynucleotides are linked by a substitute linkage, e.g., a phosphorothioate linkage.

In certain embodiments, most or all of the nucleotides beyond the guide sequence (2′-modified or not) are linked by phosphorothioate linkages. Such constructs tend to have improved pharmacokinetics due to their higher affinity for serum proteins. The phosphorothioate linkages in the non-guide sequence portion of the polynucleotide generally do not interfere with guide strand activity, once the latter is loaded into RISC.

Guide sequences of the present invention may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

### III. Synthesis

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In certain embodiments, chemical synthesis may be used to selectively produce the subject double-stranded constructs, with minimal contamination by the alternative mini hairpin structures formed from the single-stranded polynucleotide.

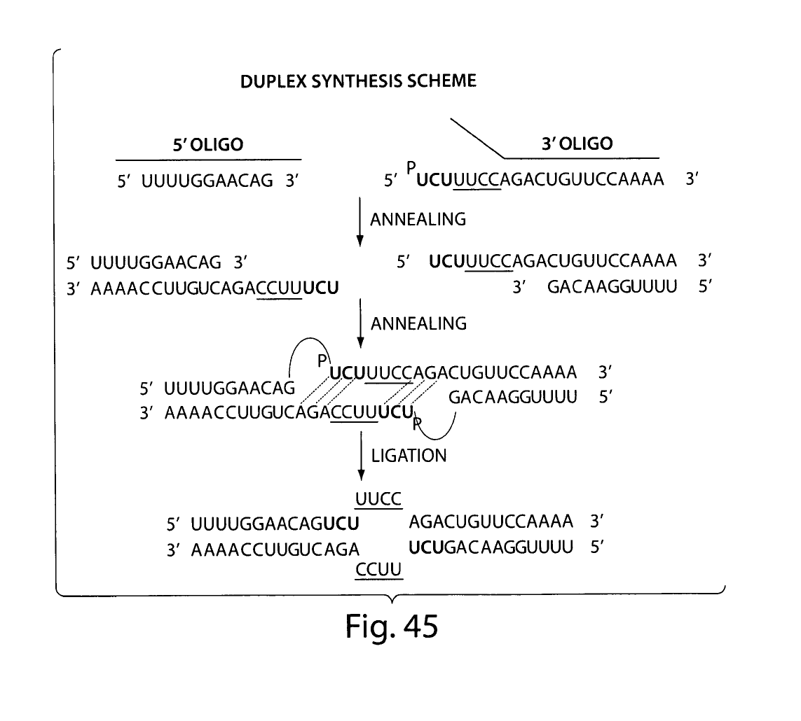

According to this embodiment, as shown in the schematic drawing in FIG. 45, two fragments of the single-stranded polynucleotide are synthesized. The fragment corresponding to the 5′-end of the single-stranded polynucleotide is shorter—less than the duplex stem length. In certain embodiments, the short fragment has at least about 7-10 nucleotides, preferably >8 nucleotides. The fragment corresponding to the 3′-end of the single-stranded polynucleotide is longer, and includes (from its 5′-end) at least 2-3 bases from the 3′-end of the duplex stem, the linker sequence that forms the loop/bulge, and the entire sequence of the second stem region. The 5′-end of the longer fragment (the 3′-end fragment in this case) is phosphorylated, while the 5′-end of the shorter fragment is not phosphorylated.

In this embodiments, the 5′-most 2-3 nucleotides of the longer fragment may contain one or more chemical modifications or nucleotide analogs (such as LNA) that have increased binding affinity to their respective base-pairing partners.

The longer and shorter oligonucleotide fragments are then mixed together to allow annealing under appropriate conditions. Annealing can be done by titrating one or the other fragment to completion. This can be achieved by, for example, monitoring the presence of the various monomer fragments by HPLC. One intermediate annealing product is a double-stranded structure having a blunt end and an end with overhang (5′ overhang in this case). The 5′-end of the overhang is phosphorylated, while the 5′-end of the blunt end is not phosphorylated.

The intermediate annealing product can further anneal with one another to produce the final annealing product—the subject double-stranded polynucleotide constructs with two duplex stems flanking the central loop/bulge. This annealing step may be further facilitated by the presence (if any), at the 5′-overhang of the intermediate annealing product, of modified nucleotides or nucleotide analogs having enhanced base-pairing affinity.

The final annealing product can be ligated together by using a ligase, such as T4 DNA ligase (which also catalyzes the formation of a phosphodiester bond between juxtaposed 5′ phosphate and 3′ hydroxyl termini in a duplex RNA), under appropriate conditions. Ligation of the desired final annealing product is favored over other potential ligation products (such as head-to-tail tandem ligation products shown in FIG. 45), partly because of the base-pairing in the correct ligation product.

Similar approach can be used to synthesize the subject double-stranded polynucleotide construct if the shorter fragment is at the 3′-end of the single-stranded polynucleotide, while the longer fragment (including the linker sequence) is at the 5′-end of the single-stranded polynucleotide. In this case, the intermediate annealing product has a 3′-overhang, and the 5′-end of the shorter fragment is phosphorylated. The base-pairing enhancing modified nucleotides or nucleotide analogs are at the 3′-end of the longer fragment.

Thus in addition to the method described herein wherein two identical single-stranded polynucleotides may be used to produce the subject double-stranded polynucleotide, the invention further provides an alternative synthesis method, comprising: (1) providing a 5′-end fragment of the single-stranded polynucleotide and a 3′-end fragment of the single-stranded polynucleotide, wherein the 5′-end fragment does not have 5′-end phosphorylation and the 3′-end fragment has 5′-end phosphorylation; wherein the longer fragment comprises a full-length stem region sequence, the linker sequence, and at least 2-3 nucleotides corresponding to the other stem region sequence; (2) allowing the 5′-end fragment and the 3′-end fragment to anneal; and, (3) ligating the final annealing product to generate the subject double-stranded polynucleotide construct.

In general, chemical synthesis can be used for the synthesis of the modified polynucleotides (in the embodiments described above or other unrelated embodiments). Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; and U.S. Pat. No. 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J. Chrom. 599:35.

Other exemplary synthesis techniques are well known in the art (see, e.g., Sambrook et al., Molecular Cloning: a Laboratory Manual, Second Edition (1989); DNA Cloning, Volumes I and II (DN Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject RNAi constructs or at least portions thereof are transcribed from expression vectors encoding the subject constructs. Any art recognized vectors may be use for this purpose. The transcribed RNAi constructs may be isolated and purified, before desired modifications (such as replacing an unmodified sense strand with a modified one, etc.) are carried out.

### IV. Delivery/Carrier

### Uptake of Oligonucleotides by Cells

Oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In a preferred embodiment, the oligonucleotide compositions of the invention are contacted with human cells.

Oligonucleotide compositions of the invention can be contacted with cells in vitro, e.g., in a test tube or culture dish, (and may or may not be introduced into a subject) or in vivo, e.g., in a subject such as a mammalian subject. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet. 2003 Jan. 19:9; Reichhart J M et al. Genesis. 2002. 34(1-2):1604, Yu et al. 2002. Proc. Natl. Acad. Sci. USA 99:6047; Sui et al. 2002. Proc. Natl. Acad. Sci. USA 99:5515) viruses, polyamine or polycation conjugates using compounds such as polylysine, protamine, or Ni, N12-bis(ethyl) spermine (see, e.g., Bartzatt, R. et al. 1989. Biotechnol. Appl. Biochem. 11:133; Wagner E. et al. 1992. Proc. Natl. Acad. Sci. 88:4255).

In certain embodiments, the constructs of the invention may be delivered by using various beta-glucan containing particles, such as those described in US 2005/0281781 A 1, WO 2006/007372, and WO 2007/050643 (all incorporated herein by reference). In certain embodiments, the beta-glucan particle is derived from yeast. In certain embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine), etc.

Such beta-glucan based delivery system may be formulated for oral delivery, where the orally delivered beta-glucan/subject constructs may be engulfed by macrophages or other related phagocytic cells, which may in turn release the subject constructs in selected in vivo sites. Alternatively or in addition, the subject constructs may change the expression of certain macrophage target genes.

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Conjugating Agents

Conjugating agents bind to the oligonucleotide in a covalent manner. In one embodiment, oligonucleotides can be derivatized or chemically modified by binding to a conjugating agent to facilitate cellular uptake. For example, covalent linkage of a cholesterol moiety to an oligonucleotide can improve cellular uptake by 5- to 10-fold which in turn improves DNA binding by about 10-fold (Boutorin et al., 1989, FEBS Letters 254:129-132). Conjugation of octyl, dodecyl, and octadecyl residues enhances cellular uptake by 3-, 4-, and 10-fold as compared to unmodified oligonucleotides (Vlassov et al., 1994, Biochimica et Biophysica Acta 1197:95-108). Similarly, derivatization of oligonucleotides with poly-L-lysine can aid oligonucleotide uptake by cells (Schell, 1974, Biochem. Biophys. Acta 340:323, and Lemaitre et al., 1987, Proc. Natl. Acad. Sci. USA 84:648).

Certain protein carriers can also facilitate cellular uptake of oligonucleotides, including, for example, serum albumin, nuclear proteins possessing signals for transport to the nucleus, and viral or bacterial proteins capable of cell membrane penetration. Therefore, protein carriers are useful when associated with or linked to the oligonucleotides. Accordingly, the present invention provides for derivatization of oligonucleotides with groups capable of facilitating cellular uptake, including hydrocarbons and non-polar groups, cholesterol, long chain alcohols (i.e., hexanol), poly-L-lysine and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes, and steroids. A major advantage of using conjugating agents is to increase the initial membrane interaction that leads to a greater cellular accumulation of oligonucleotides.

Other conjugating agents include various vitamins, such as fat soluble vitamins, which may be used as a conjugate to deliver RNAi constructs specifically into adipose tissue—the primary location where these vitamins are stored. These vitamin-based conjugating agents may be especially useful for targeting certain metabolic disease targets, such as diabetes/obesity. Of the fat soluble vitamins, such as vitamins A, D, E, K, etc., vitamin K may be preferred in some embodiments, as there is no known upper intake level (although large doses could lead to breakdown of red blood cells and possibly to liver disease). In comparison, vitamins A and D have more defined toxicity and established upper intake levels.

In certain embodiments, gamma carboxyglutamic acid residues may be conjugated to the subject RNAi constructs to increased their membrane stickiness, and/or to slow clearance and improve general uptake (infra).

Certain conjugating agents that may be used with the instant constructs include those described in WO04048545A2 and US20040204377A1 (all incorporated herein by their entireties), such as a Tat peptide, a sequence substantially similar to the sequence of SEQ ID NO: 12 of WO04048545A2 and US20040204377A1, a homeobox (hox) peptide, a MTS, VP22, MPG, at least one dendrimer (such as PAMAM), etc.

Other conjugating agents that may be used with the instant constructs include those described in WO07089607A2 (incorporated herein), which describes various nanotransporters and delivery complexes for use in delivery of nucleic acid molecules (such as the subject dsRNA constructs) and/or other pharmaceutical agents in vivo and in vitro. Using such delivery complexes, the subject dsRNA can be delivered while conjugated or associated with a nanotransporter comprising a core conjugated with at least one functional surface group. The core may be a nanoparticle, such as a dendrimer (e.g., a polylysine dendrimer). The core may also be a nanotube, such as a single walled nanotube or a multi-walled nanotube. The functional surface group is at least one of a lipid, a cell type specific targeting moiety, a fluorescent molecule, and a charge controlling molecule. For example, the targeting moiety may be a tissue-selective peptide. The lipid may be an oleoyl lipid or derivative thereof. Exemplary nanotransporter include NOP-7 or HBOLD.

### Encapsulating Agents

Encapsulating agents entrap oligonucleotides within vesicles. In another embodiment of the invention, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present invention may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

### Complexing Agents

Complexing agents bind to the oligonucleotides of the invention by a strong but non-covalent attraction (e.g., an electrostatic, van der Waals, pi-stacking, etc. interaction). In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminiurn trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. No. 4,235,871; U.S. Pat. Nos. 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93:3176). Cationic lipids and other complexing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. U.S.A. 93:3176). In one embodiment, a composition for delivering oligonucleotides of the invention comprises a number of arginine, lysine, histidine or ornithine residues linked to a lipophilic moiety (see e.g., U.S. Pat. No. 5,777,153).

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, a composition for delivering oligonucleotides of the invention comprises a natural or synthetic polypeptide having one or more gamma carboxyglutamic acid residues, or γ-Gla residues. These gamma carboxyglutamic acid residues may enable the polypeptide to bind to each other and to membrane surfaces. In other words, a polypeptide having a series of γ-Gla may be used as a general delivery modality that helps an RNAi construct to stick to whatever membrane to which it comes in contact. This may at least slow RNAi constructs from being cleared from the blood stream and enhance their chance of homing to the target.

The gamma carboxyglutamic acid residues may exist in natural proteins (for example, prothrombin has 10 γ-Gla residues). Alternatively, they can be introduced into the purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated/fine tuned to achieve different levels of “stickiness” of the polypeptide.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J. Cell Biol. 128:919). In another embodiment, a Boc-Cys-(Npys)OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleomonomer and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀alkenyl chains, C₂-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see, e.g., Smith et al. Biochem J 1991. 276: 417-2).

In one embodiment, oligonucleotides of the invention are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

### Targeting Agents

The delivery of oligonucleotides can also be improved by targeting the oligonucleotides to a cellular receptor. The targeting moieties can be conjugated to the oligonucleotides or attached to a carrier group (i.e., poly(L-lysine) or liposomes) linked to the oligonucleotides. This method is well suited to cells that display specific receptor-mediated endocytosis.

For instance, oligonucleotide conjugates to 6-phosphomannosylated proteins are internalized 20-fold more efficiently by cells expressing mannose 6-phosphate specific receptors than free oligonucleotides. The oligonucleotides may also be coupled to a ligand for a cellular receptor using a biodegradable linker. In another example, the delivery construct is mannosylated streptavidin which forms a tight complex with biotinylated oligonucleotides. Mannosylated streptavidin was found to increase 20-fold the internalization of biotinylated oligonucleotides. (Vlassov et al. 1994. Biochimica et Biophysica Acta 1197:95-108).

In addition specific ligands can be conjugated to the polylysine component of polylysine-based delivery systems. For example, transferrin-polylysine, adenovirus-polylysine, and influenza virus hemagglutinin HA-2 N-terminal fusogenic peptides-polylysine conjugates greatly enhance receptor-mediated DNA delivery in eucaryotic cells. Mannosylated glycoprotein conjugated to poly(L-lysine) in aveolar macrophages has been employed to enhance the cellular uptake of oligonucleotides. Liang et al. 1999. Pharmazie 54:559-566.

Because malignant cells have an increased need for essential nutrients such as folic acid and transferrin, these nutrients can be used to target oligonucleotides to cancerous cells. For example, when folic acid is linked to poly(L-lysine) enhanced oligonucleotide uptake is seen in promyelocytic leukemia (HL-60) cells and human melanoma (M-14) cells. Ginobbi et al. 1997. Anticancer Res. 17:29. In another example, liposomes coated with maleylated bovine serum albumin, folic acid, or ferric protoporphyrin IX, show enhanced cellular uptake of oligonucleotides in murine macrophages, KB cells, and 2.2.15 human hepatoma cells. Liang et al. 1999. Pharmazie 54:559-566.

Liposomes naturally accumulate in the liver, spleen, and reticuloendothelial system (so-called, passive targeting). By coupling liposomes to various ligands such as antibodies are protein A, they can be actively targeted to specific cell populations. For example, protein A-bearing liposomes may be pretreated with H-2K specific antibodies which are targeted to the mouse major histocompatibility complex-encoded H-2K protein expressed on L cells. (Vlassov et al. 1994. Biochimica et Biophysica Acta 1197:95-108).

Other in vitro and/or in vivo delivery of RNAi reagents are known in the art, and can be used to deliver the subject RNAi constructs. See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2, just to name a few (all incorporated by reference).

### V. Administration

The optimal course of administration or delivery of the oligonucleotides may vary depending upon the desired result and/or on the subject to be treated. As used herein “administration” refers to contacting cells with oligonucleotides and can be performed in vitro or in vivo. The dosage of oligonucleotides may be adjusted to optimally reduce expression of a protein translated from a target nucleic acid molecule, e.g., as measured by a readout of RNA stability or by a therapeutic response, without undue experimentation.

For example, expression of the protein encoded by the nucleic acid target can be measured to determine whether or not the dosage regimen needs to be adjusted accordingly. In addition, an increase or decrease in RNA or protein levels in a cell or produced by a cell can be measured using any art recognized technique. By determining whether transcription has been decreased, the effectiveness of the oligonucleotide in inducing the cleavage of a target RNA can be determined.

Any of the above-described oligonucleotide compositions can be used alone or in conjunction with a pharmaceutically acceptable carrier. As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types.

Moreover, the present invention provides for administering the subject oligonucleotides with an osmotic pump providing continuous infusion of such oligonucleotides, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, e.g., parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

Pharmaceutical preparations for topical administration include transdermal patches, ointments, lotions, creams, gels, drops, sprays, suppositories, liquids and powders. In addition, conventional pharmaceutical carriers, aqueous, powder or oily bases, or thickeners may be used in pharmaceutical preparations for topical administration.

Pharmaceutical preparations for oral administration include powders or granules, suspensions or solutions in water or non-aqueous media, capsules, sachets or tablets. In addition, thickeners, flavoring agents, diluents, emulsifiers, dispersing aids, or binders may be used in pharmaceutical preparations for oral administration.

For transmucosal or transdermal administration, penetrants appropriate to the barrier to be permeated are used in the formulation. Such penetrants are known in the art, and include, for example, for transmucosal administration bile salts and fusidic acid derivatives, and detergents. Transmucosal administration may be through nasal sprays or using suppositories. For oral administration, the oligonucleotides are formulated into conventional oral administration forms such as capsules, tablets, and tonics. For topical administration, the oligonucleotides of the invention are formulated into ointments, salves, gels, or creams as known in the art.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described oligonucleotides may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the oligonucleotide to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the oligonucleotide at the lymph node. The oligonucleotide can be modified to diffuse into the cell, or the liposome can directly participate in the delivery of either the unmodified or modified oligonucleotide into the cell.

The chosen method of delivery will result in entry into cells. Preferred delivery methods include liposomes (10-400 nm), hydrogels, controlled-release polymers, and other pharmaceutically applicable vehicles, and microinjection or electroporation (for ex vivo treatments).

The pharmaceutical preparations of the present invention may be prepared and formulated as emulsions. Emulsions are usually heterogeneous systems of one liquid dispersed in another in the form of droplets usually exceeding 0.1 μm in diameter. The emulsions of the present invention may contain excipients such as emulsifiers, stabilizers, dyes, fats, oils, waxes, fatty acids, fatty alcohols, fatty esters, humectants, hydrophilic colloids, preservatives, and anti-oxidants may also be present in emulsions as needed. These excipients may be present as a solution in either the aqueous phase, oily phase or itself as a separate phase.

Examples of naturally occurring emulsifiers that may be used in emulsion formulations of the present invention include lanolin, beeswax, phosphatides, lecithin and acacia. Finely divided solids have also been used as good emulsifiers especially in combination with surfactants and in viscous preparations. Examples of finely divided solids that may be used as emulsifiers include polar inorganic solids, such as heavy metal hydroxides, nonswelling clays such as bentonite, attapulgite, hectorite, kaolin, montrnorillonite, colloidal aluminum silicate and colloidal magnesium aluminum silicate, pigments and nonpolar solids such as carbon or glyceryl tristearate.

Examples of preservatives that may be included in the emulsion formulations include methyl paraben, propyl paraben, quaternary ammonium salts, benzalkonium chloride, esters of p-hydroxybenzoic acid, and boric acid. Examples of antioxidants that may be included in the emulsion formulations include free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of oligonucleotides are formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both oil/water and water/oil) have been proposed to enhance the oral bioavailability of drugs.

Microemulsions offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11:1385; Ho et al., J. Pharm. Sci., 1996, 85:138-143). Microemulsions have also been effective in the transdermal delivery of active components in both cosmetic and pharmaceutical applications. It is expected that the microemulsion compositions and formulations of the present invention will facilitate the increased systemic absorption of oligonucleotides from the gastrointestinal tract, as well as improve the local cellular uptake of oligonucleotides within the gastrointestinal tract, vagina, buccal cavity and other areas of administration.

In an embodiment, the present invention employs various penetration enhancers to affect the efficient delivery of nucleic acids, particularly oligonucleotides, to the skin of animals. Even non-lipophilic drugs may cross cell membranes if the membrane to be crossed is treated with a penetration enhancer. In addition to increasing the diffusion of non-lipophilic drugs across cell membranes, penetration enhancers also act to enhance the permeability of lipophilic drugs.

Five categories of penetration enhancers that may be used in the present invention include: surfactants, fatty acids, bile salts, chelating agents, and non-chelating non-surfactants. Other agents may be utilized to enhance the penetration of the administered oligonucleotides include: glycols such as ethylene glycol and propylene glycol, pyrrols such as 2-15 pyrrol, azones, and terpenes such as limonene, and menthone.

The oligonucleotides, especially in lipid formulations, can also be administered by coating a medical device, for example, a catheter, such as an angioplasty balloon catheter, with a cationic lipid formulation. Coating may be achieved, for example, by dipping the medical device into a lipid formulation or a mixture of a lipid formulation and a suitable solvent, for example, an aqueous-based buffer, an aqueous solvent, ethanol, methylene chloride, chloroform and the like. An amount of the formulation will naturally adhere to the surface of the device which is subsequently administered to a patient, as appropriate. Alternatively, a lyophilized mixture of a lipid formulation may be specifically bound to the surface of the device. Such binding techniques are described, for example, in K. Ishihara et al., Journal of Biomedical Materials Research, Vol. 27, pp. 1309-1314 (1993), the disclosures of which are incorporated herein by reference in their entirety.

The useful dosage to be administered and the particular mode of administration will vary depending upon such factors as the cell type, or for in vivo use, the age, weight and the particular animal and region thereof to be treated, the particular oligonucleotide and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligonucleotides, the amount of lipid compound that is administered can vary and generally depends upon the amount of oligonucleotide agent being administered. For example, the weight ratio of lipid compound to oligonucleotide agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligonucleotide agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the oligonucleotide is administered in a form in which any toxic effects are outweighed by the therapeutic effects of the oligonucleotide. In one embodiment, oligonucleotides can be administered to subjects. Examples of subjects include mammals, e.g., humans and other primates; cows, pigs, horses, and farming (agricultural) animals; dogs, cats, and other domesticated pets; mice, rats, and transgenic non-human animals.

Administration of an active amount of an oligonucleotide of the present invention is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically-modified oligonucleotides, e.g., with modification of the phosphate backbone, may require different dosing.

The exact dosage of an oligonucleotide and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regimen may be adjusted to provide the optimum therapeutic response. For example, the oligonucleotide may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

### VI. Assays of Oligonucleotide Stability

Preferably, the subject polynucleotide constructs (oligonucleotides) are stabilized, i.e., substantially resistant to endonuclease and exonuclease degradation. An oligonucleotide is defined as being substantially resistant to nucleases when it is at least about 3-fold more resistant to attack by an endogenous cellular nuclease, and is highly nuclease resistant when it is at least about 6-fold more resistant than a corresponding, single-stranded oligonucleotide. This can be demonstrated by showing that the oligonucleotides of the invention are substantially resistant to nucleases using techniques which are known in the art.

One way in which substantial stability can be demonstrated is by showing that the oligonucleotides of the invention function when delivered to a cell, e.g., that they reduce transcription or translation of target nucleic acid molecules, e.g., by measuring protein levels or by measuring cleavage of mRNA. Assays which measure the stability of target RNA can be performed at about 24 hours post-transfection (e.g., using Northern blot techniques, RNase Protection Assays, or QC-PCR assays as known in the art). Alternatively, levels of the target protein can be measured. Preferably, in addition to testing the RNA or protein levels of interest, the RNA or protein levels of a control, non-targeted gene will be measured (e.g., actin, or preferably a control with sequence similarity to the target) as a specificity control. RNA or protein measurements can be made using any art-recognized technique. Preferably, measurements will be made beginning at about 16-24 hours post transfection. (M. Y. Chiang, et al. 1991. J Biol. Chem. 266:18162-71; T. Fisher, et al. 1993. Nucleic Acids Research. 21 3857).

The ability of an oligonucleotide composition of the invention to inhibit protein synthesis can be measured using techniques which are known in the art, for example, by detecting an inhibition in gene transcription or protein synthesis. For example, Nuclease S1 mapping can be performed. In another example, Northern blot analysis can be used to measure the presence of RNA encoding a particular protein. For example, total RNA can be prepared over a cesium chloride cushion (see, e.g., Ausebel et al., 1987. Current Protocols in Molecular Biology (Greene & Wiley, New York)). Northern blots can then be made using the RNA and probed (see, e.g., Id.). In another example, the level of the specific mRNA produced by the target protein can be measured, e.g., using PCR. In yet another example, Western blots can be used to measure the amount of target protein present. In still another embodiment, a phenotype influenced by the amount of the protein can be detected. Techniques for performing Western blots are well known in the art, see, e.g., Chen et al. J. Biol. Chem. 271:28259.

In another example, the promoter sequence of a target gene can be linked to a reporter gene and reporter gene transcription (e.g., as described in more detail below) can be monitored. Alternatively, oligonucleotide compositions that do not target a promoter can be identified by fusing a portion of the target nucleic acid molecule with a reporter gene so that the reporter gene is transcribed. By monitoring a change in the expression of the reporter gene in the presence of the oligonucleotide composition, it is possible to determine the effectiveness of the oligonucleotide composition in inhibiting the expression of the reporter gene. For example, in one embodiment, an effective oligonucleotide composition will reduce the expression of the reporter gene.

A “reporter gene” is a nucleic acid that expresses a detectable gene product, which may be RNA or protein. Detection of mRNA expression may be accomplished by Northern blotting and detection of protein may be accomplished by staining with antibodies specific to the protein. Preferred reporter genes produce a readily detectable product. A reporter gene may be operably linked with a regulatory DNA sequence such that detection of the reporter gene product provides a measure of the transcriptional activity of the regulatory sequence. In preferred embodiments, the gene product of the reporter gene is detected by an intrinsic activity associated with that product. For instance, the reporter gene may encode a gene product that, by enzymatic activity, gives rise to a detectable signal based on color, fluorescence, or luminescence. Examples of reporter genes include, but are not limited to, those coding for chloramphenicol acetyl transferase (CAT), luciferase, beta-galactosidase, and alkaline phosphatase.

One skilled in the art would readily recognize numerous reporter genes suitable for use in the present invention. These include, but are not limited to, chloramphenicol acetyltransferase (CAT), luciferase, human growth hormone (hGH), and beta-galactosidase. Examples of such reporter genes can be found in F. A. Ausubel et al., Eds., Current Protocols in Molecular Biology, John Wiley & Sons, New York, (1989). Any gene that encodes a detectable product, e.g., any product having detectable enzymatic activity or against which a specific antibody can be raised, can be used as a reporter gene in the present methods.

One reporter gene system is the firefly luciferase reporter system. (Gould, S. J., and Subramani, S. 1988. Anal. Biochem., 7:404-408 incorporated herein by reference). The luciferase assay is fast and sensitive. In this assay, a lysate of the test cell is prepared and combined with ATP and the substrate luciferin. The encoded enzyme luciferase catalyzes a rapid, ATP dependent oxidation of the substrate to generate a light-emitting product. The total light output is measured and is proportional to the amount of luciferase present over a wide range of enzyme concentrations.

CAT is another frequently used reporter gene system; a major advantage of this system is that it has been an extensively validated and is widely accepted as a measure of promoter activity. (Gorman C. M., Moffat, L. F., and Howard, B. H. 1982. Mol. Cell. Biol., 2:1044-1051). In this system, test cells are transfected with CAT expression vectors and incubated with the candidate substance within 2-3 days of the initial transfection. Thereafter, cell extracts are prepared. The extracts are incubated with acetyl CoA and radioactive chloramphenicol. Following the incubation, acetylated chloramphenicol is separated from nonacetylated form by thin layer chromatography. In this assay, the degree of acetylation reflects the CAT gene activity with the particular promoter.

Another suitable reporter gene system is based on immunologic detection of hGH. This system is also quick and easy to use. (Selden, R., Burke-Howie, K. Rowe, M. E., Goodman, H. M., and Moore, D. D. (1986), Mol. Cell, Biol., 6:3173-3179 incorporated herein by reference). The hGH system is advantageous in that the expressed hGH polypeptide is assayed in the media, rather than in a cell extract. Thus, this system does not require the destruction of the test cells. It will be appreciated that the principle of this reporter gene system is not limited to hGH but rather adapted for use with any polypeptide for which an antibody of acceptable specificity is available or can be prepared.

In one embodiment, nuclease stability of a double-stranded oligonucleotide of the invention is measured and compared to a control, e.g., an RNAi molecule typically used in the art (e.g., a duplex oligonucleotide of less than 25 nucleotides in length and comprising 2 nucleotide base overhangs) or an unmodified RNA duplex with blunt ends.

### VII. Therapeutic use

By inhibiting the expression of a gene, the oligonucleotide compositions of the present invention can be used to treat any disease involving the expression of a protein. Examples of diseases that can be treated by oligonucleotide compositions, just to illustrate, include: cancer, retinopathies, autoimmune diseases, inflammatory diseases (i.e., ICAM-1 related disorders, Psoriasis, Ulcerative Colitis, Crohn's disease), viral diseases (i.e., HIV, Hepatitis C), miRNA disorders, and cardiovascular diseases.

In one embodiment, in vitro treatment of cells with oligonucleotides can be used for ex vivo therapy of cells removed from a subject (e.g., for treatment of leukemia or viral infection) or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to evaluate gene function, to study gene regulation and protein synthesis or to evaluate improvements made to oligonucleotides designed to modulate gene expression or protein synthesis. In vivo treatment of cells can be useful in certain clinical settings where it is desirable to inhibit the expression of a protein. There are numerous medical conditions for which antisense therapy is reported to be suitable (see, e.g., U.S. Pat. No. 5,830,653) as well as respiratory syncytial virus infection (WO 95/22,553) influenza virus (WO 94/23,028), and malignancies (WO 94/08,003). Other examples of clinical uses of antisense sequences are reviewed, e.g., in Glaser. 1996. Genetic Engineering News 16:1. Exemplary targets for cleavage by oligonucleotides include, e.g., protein kinase Ca, ICAM-1, c-raf kinase, p53, c-myb, and the bcr/abl fusion gene found in chronic myelogenous leukemia.

The subject nucleic acids can be used in RNAi-based therapy in any animal having RNAi pathway, such as human, non-human primate, non-human mammal, non-human vertebrates, rodents (mice, rats, hamsters, rabbits, etc.), domestic livestock animals, pets (cats, dogs, etc.), Xenopus, fish, insects (Drosophila, etc.), and worms (C. elegans), etc.

### EXAMPLES

### Example I

### Dose Response Attenuation of PPIB in HEK293 Cells

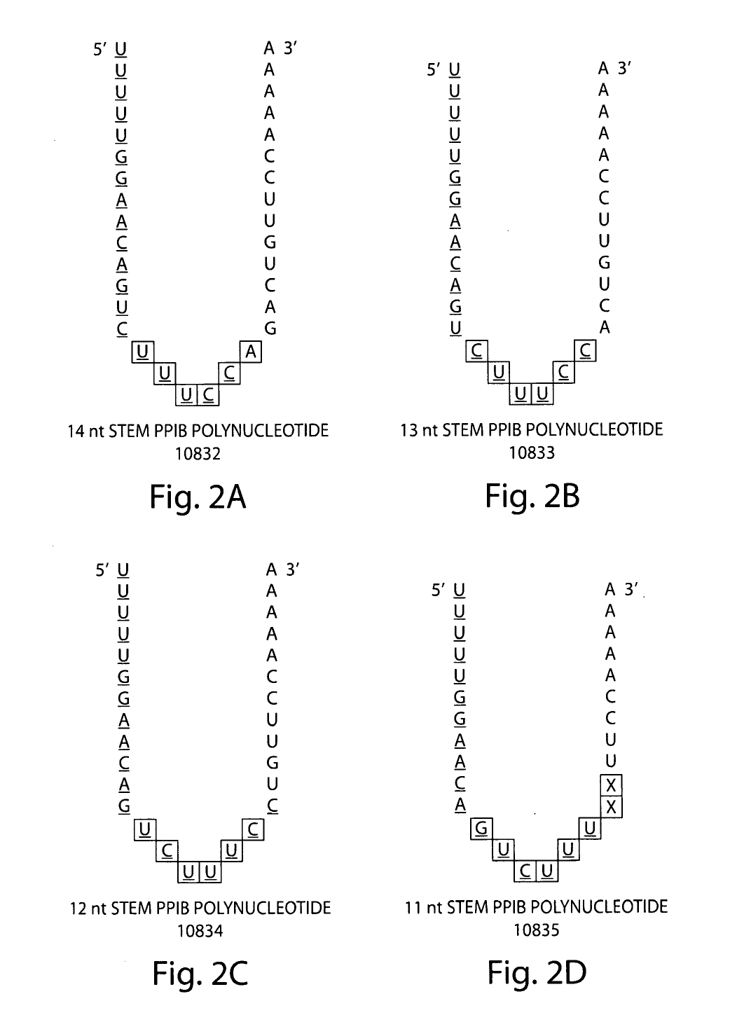

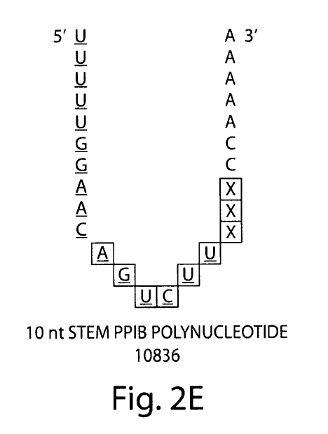

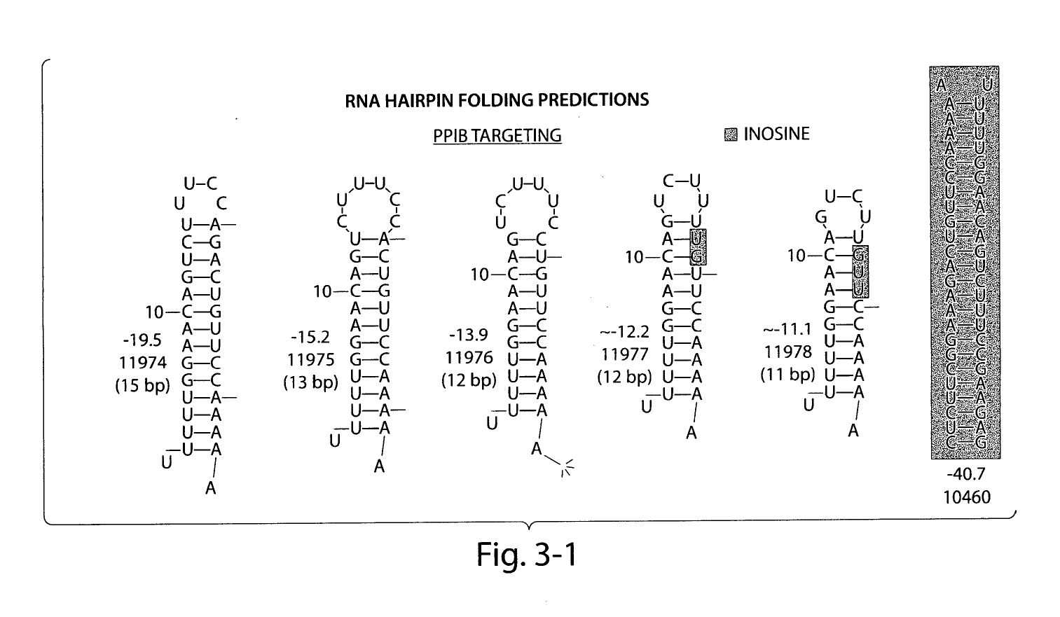

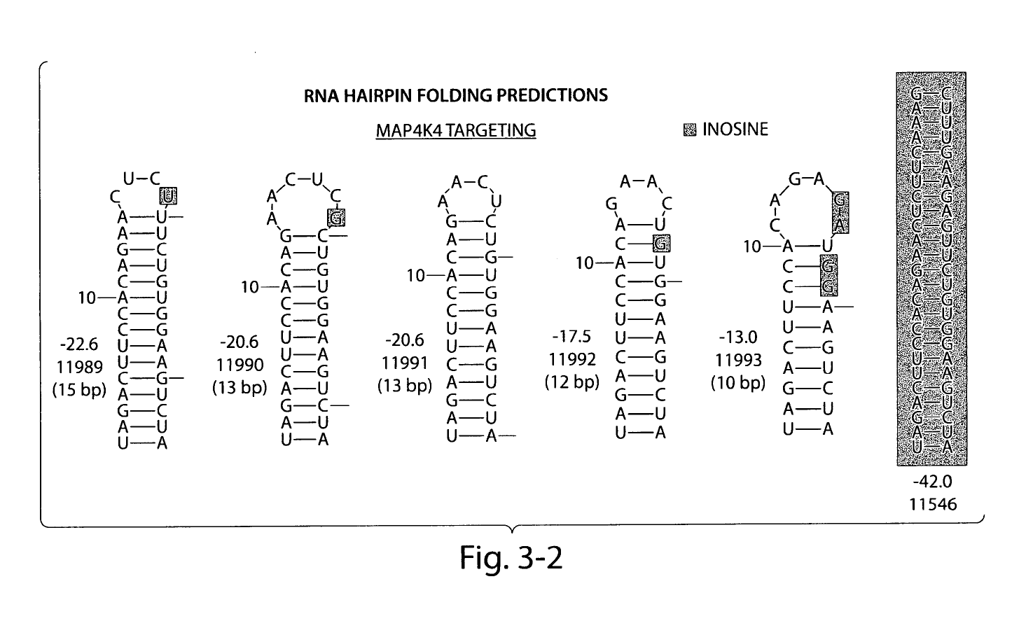

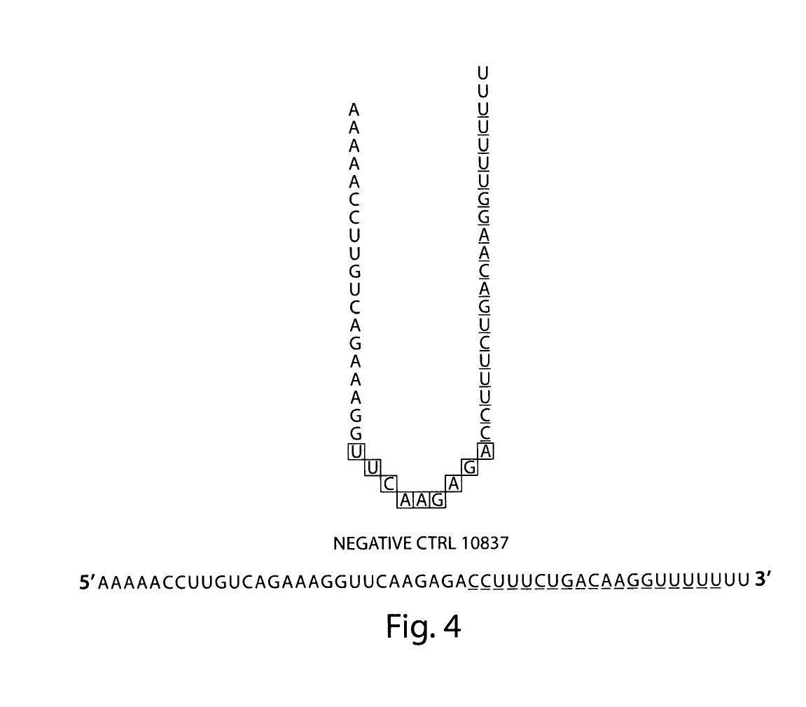

To demonstrate the efficacy of the constructs of the present invention, HEK293 cells were transfected with constructs prepared from single-stranded polynucleotide 10833 (SEQ ID NO: 2) and 10834 (SEQ ID NO: 3), which are shown in FIGS. 2B and 2C. Positive and negative controls were also included. As shown and described in FIG. 4, construct prepared from single-stranded polynucleotide 10837 (SEQ ID NO: 6) was used as a negative control. In addition, an RNA construct previously shown to be effective in reducing PPIB expression was used in this experiment as a positive control. This structure, denoted as construct 10460 (5′P-mCmUmCmUUCGGAAAGACUGUUCCAmAmAmAmA-3′, SEQ ID NO: 7) in this example, contains 2′-O-methyl modified bases on 4 of the outermost positions at both ends of the sequence.

All constructs were transfected into HEK293 cells using the LIPOFECTAMINE™ RNAiMAX reagent (Invitrogen Corp., Carlsbad, Calif.) according to manufacture's instructions. In brief, RNA was diluted to a 12× concentration and then combined with a 12× concentration of LIPOFECTAMINE™ RNAiMAX to complex. The RNA and transfection reagent were allowed to complex at room temperature for 20 minutes to yield a 6× concentration. While complexing, HEK293 cells were washed, trypsinized and counted. The cells were diluted to a concentration recommended by the manufacturer which was at 1×10⁵ cells/ml. When RNA has completed complexing with the RNAiMAX transfection reagent, 20 μl of the complexes were added to the appropriate well of the 96-well plate in triplicate. Cells were added to each well (100 μl volume) to make the final cell count per well at 1×10⁴ cells/well. The volume of cells diluted the 6× concentration of complex to 1× which was equal to 50, 25, or 10 nM. Cells were incubated for 48 hours under normal growth conditions. After 48 hour incubation, cells were lysed and gene silencing activity was measured using the Panomics QUANTIGENE™ assay which employs bDNA hybridization technology. The assay was carried out according to manufacturer's instructions.

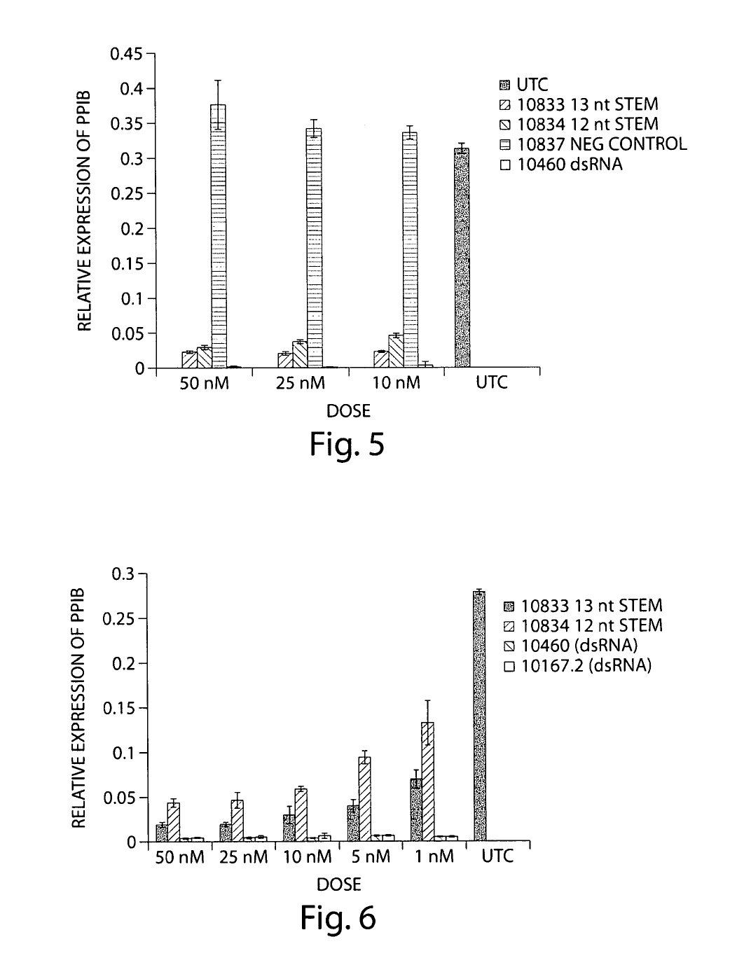

FIG. 5 illustrates the relative expression of PPIB remaining after transfection of each construct, with the exception of UTC (untransfected control), at the indicated concentrations. As compared to the negative control 10837 and UTC, constructs 10833 and 10834 dramatically decrease PPIB expression even at 10 nM. The transfection conditions are detailed below.

### Detailed Protocol:

Active RNAi construct stocks at 100 or 10 μM were used to make a 3 μM working dilution in RNA Buffer (1:33.333 dilution) by mixing 30 μl of a 10 μM stock+70 μl RNA Buffer. Diluted RNAi compound plates were prepared in a 96 well plate of 0.2 mL PCR tubes as follows:

a. 50 nM Condition (dilute RNA compound to 12× or to 600 nM in OptiMEM, Reduced Serum Medium, Invitrogen Corp., Carlsbad, Calif.):
i. Add 80 μl of OptiMEM to each well
ii. Add 20 μl of working stock RNAi compound (3 μM) to each well with OptiMEM

b. 25 nM Condition (dilute RNA to make 12× or to 300 nM in OptiMEM):
i. Add 50 μl of OptiMEM to each well
ii. Add 50 μl from 60 nM RNAi active compound (50 nM) to each well with OptiMEM

c. 10 nM Condition (dilute RNA compound to 12× or to 120 nM in OptiMEM):
i. Add 30 μl of OptiMEM to each well
ii. Add 20 μl from 12 nM RNAi active compound (25 nM) to each well with OptiMEM

A bulk amount of diluted RNAiMAX and LIPOFECTAMINE™ were made as follows:

a. 1,470 μl Opti-MEM+30 μl RNAiMAX

b. 735 μl Opti-MEM+15 μl Lipofectamine 2000

c. Combine RNAiMAX and Opti-MEM, Lipofectamine and Opti-MEM, mix gently.

Diluted RNAiMAX or LIPOFECTAMINE™ (35 μl each) was added to each 0.2 ml PCR well. Then, 35 μl of diluted RNAi compound from the RNA plate (above) was also added to each well. Each well contained enough for duplicates at a given dose. Each reaction mixture was gently mixed by pipetting up and down 3 times. The mixture was allowed to complex for at least 15 minutes at room temperature. Meanwhile, a suspension of cells at 1×10⁵ cells/ml was prepared. After 15 minutes, 20 μl of complexed RNAi was added to each tissue-culture treated 96-well using a multi-channel pipettor. The prepared cell suspension was added to each reaction at a final volume of 1×10⁴ cells (100 μl of cell suspension) and allowed to incubate for 48 hours at 37° C., 10% CO₂.

### Example II

### Dose Response Experiment

To demonstrate the dose response of exemplary constructs of the present invention, HEK293 cells were transfected with constructs prepared from single-stranded sequence 10833 (SEQ ID NO: 2, the resulting construct is expected to have a loop with 6-nucleotides on each strand flanked by a 13 bp stem region on each side, forming a “bulge” in the center of the construct—see FIG. 2B for sequence) and 10834 (SEQ ID NO: 3, the resulting construct similarly having two 12 bp stem regions and a loop with 6-nucleotides on each strand, see FIG. 2C for sequence). Constructs 10460 (SEQ ID NO: 7, see above, a 25-bp double-stranded RNA with blunt ends) and 10167.2 (a 21-bp double-stranded RNA) were included as positive controls. Untransfected control (UTC) was included as a negative control. Transfections were performed as in Example I or according to the manufactures' recommendation. Each construct (except for the UTC) was transfected at a final concentration of about 50 nM, 25 nM, 10 nM, 5 nM, and 1 nM.

As shown and described in FIG. 6, both the 13-bp-stem and the 12-bp-stem constructs showed at least about 50% inhibition of the target gene PPIB expression even at the lowest tested concentration (1 nM), demonstrating the effectiveness of the subject constructs.

### Example III

### Dose Response with RISC-Free Filler

Essentially the same experiment as in Example II was repeated in Example III, with lower concentrations (0.5 nM, 0.1 nM, 0.05 nM, 0.005 nM) included and with RISC-Free filler. The RISC-Free filler is a commercially available control. In these experiments, the Filler was purchased from Dharamacon RNAi Technologies (siGENOME RISC-Free Control siRNA, Cat. No. D-001220-01-05 or D-001220-01-20). Chemical modifications in the Filler impair uptake and processing by RISC, and thus the control is not processed by RISC, making it useful to isolate cellular effects related only to siRNA transfection. Therefore, the Filler is a recommended control for performing dose response transfections to keep the nucleic acid charge to lipid ratio constant at all doses.

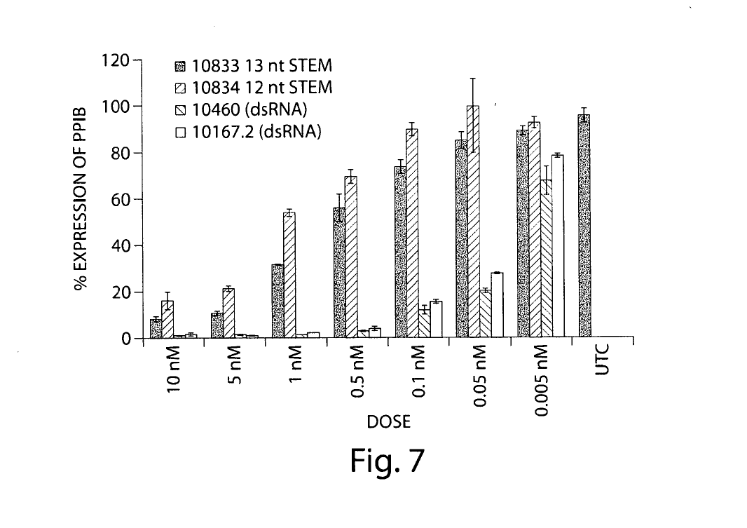

As shown in FIG. 7, both the 13-bp-stem and 12-bp-stem constructs remained partially active at sub-nano molar concentrations under the experimental conditions tested, although target gene expression was at least negative control level when both constructs are at about 0.1 nM.

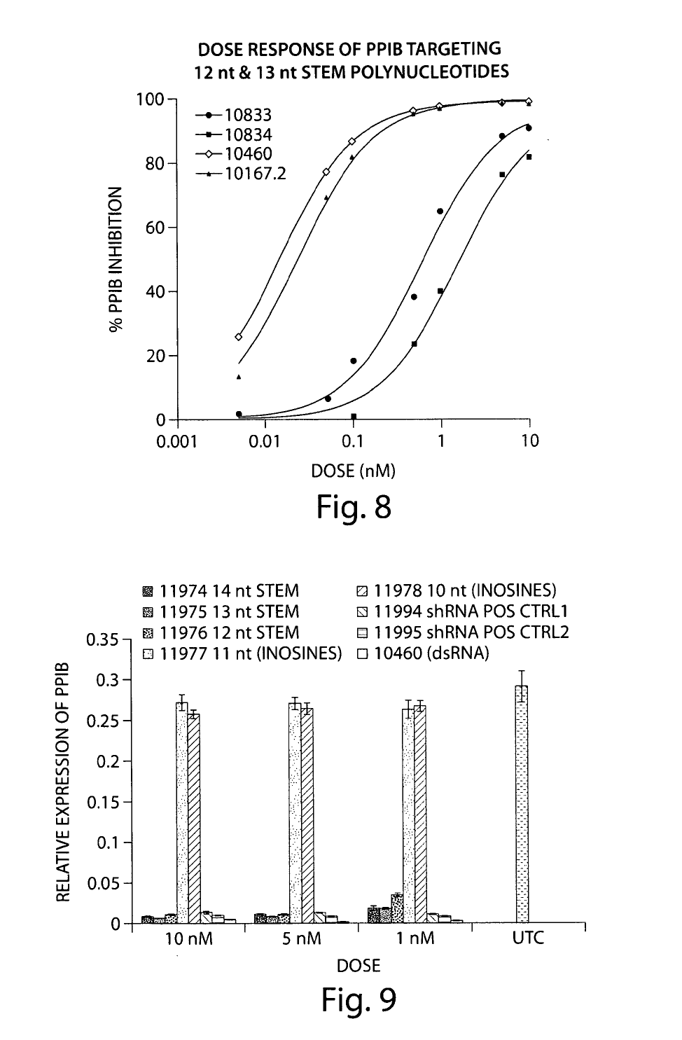

The dose response curves of this experiment were plotted in FIG. 8, and EC₅₀ values for the various constructs were calculated and listed in the table below.

| rxRNA | EC₅₀ |
|---|---|
| 10833 (13-mer-stem) | 0.599 + 0.090 |
| 10834 (12-mer-stem) | 1.540 + 0.450 |
| 10460 (25-mer dsRNA) | 0.014 + 0.001 |
| 10167.2 (21-mer dsRNA) | 0.023 + 0.002 |

It appears that the EC₅₀ values of the relatively more potent 13-mer-stem construct was about 60-fold higher than that of the 25-mer dsRNA 10460, and the EC₅₀ for the relatively less potent 12-mer-stem construct was about 110-fold higher than that of the 25-mer dsRNA.

### Example IV

### Length Requirements

To determine whether there is a minimal length requirement for the stem region in the subject constructs, five more constructs targeting the PPIB gene were used in the transfection assay as described in Example I above. The sequences and the predicted hairpin structures of the constructs are listed below. The corresponding double-stranded constructs, however, are not shown. All dsRNA controls have their respective antisense (guide) strands and sense strands shown. Structures shown in the “Intended Folding” are the initially designed foldings for the hairpin structures, while structures shown in the “Lowest Free Energy Folding” are based on predicted structures using art-recognized software that predicts nucleic acid secondary structure based on sequence information. The associated free energy was calculated using art-recognized free energy calculation software, such as the publicly available one from the University of Rochester Medical Center (publicly download: rna.urmc.rochester dot edu/rnastructure.html). See also Mathews et al. “Incorporating chemical modification constraints into a dynamic programming algorithm for prediction of RNA secondary structure,” Proceedings of the National Academy of Sciences, USA. 101: 7287-7292, 2004 (incorporated by reference). Note that, when forming the solo-rxRNA conformation, additional base pairing may occur in the loop region, since the hairpin conformation requires at least a 3-4 nt loop, and may prevent the formation of certain base-pairing that could otherwise occur when the duplex solo-rxRNA structure is adopted.

| Oligo/ | | | | |
|---|---|---|---|---|
| SEQ ID | | | Lowest Free | Intended |
| NO | Description | Sequence (5′->3′) | Energy Folding | Folding |
| 11974/ 8 | 15 nt PPIB | U.U.U.U.U.G.G.A.A.C.A.G.U.C.U.U. U.C.C.A.G.A.C.U.G.U.U.C.C.A.A.A. A.A | 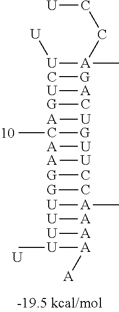 | 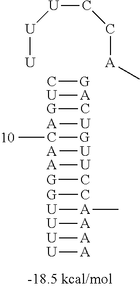 |
| 11979/ | 14 nt PPIB | SS: G.A.C.U.G.U.U.C.C.A.A.A.A.A | | |
| 9 | duplex | AS: U.U.U.U.U.G.G.A.A.C.A.G.U.C | | |
| | Control | | | |
| 11975/ 10 | 13 nt PPIB | U.U.U.U.U.G.G.A.A.C.A.G.U.C.U.U. U.C.C.A.C.U.G.U.U.C.C.A.A.A.A.A | 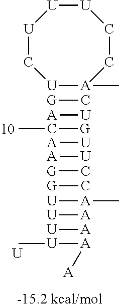 | 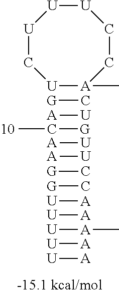 |
| 11980/ | 13 nt PPIB | SS: A.C.U.G.U.U.C.C.A.A.A.A.A | | |
| 11 | duplex | AS: U.U.U.U.U.G.G.A.A.C.A.G.U | | |
| 12 | Control | | | |
| 11976/ 13 | 12 nt PPIB | U.U.U.U.U.G.G.A.A.C.A.G.U.C.U.U. U.C.C.U.G.U.U.C.C.A.A.A.A.A | 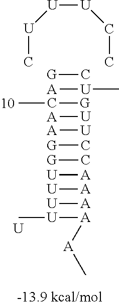 | 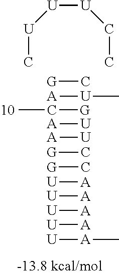 |
| 11981/ | 12 nt PPIB | SS: C.U.G.U.U.C.C.A.A.A.A.A | | |
| 14 | duplex | AS: U.U.U.U.U.G.G.A.A.C.A.G | | |
| 15 | Control | | | |
| 11977/ 16 | 12 nt PPIB | U.U.U.U.U.G.G.A.A.C.A.G.U.C.U.U. U.I.I.U.U.C.C.A.A.A.A.A | 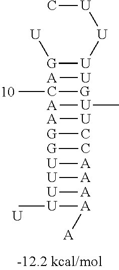 | 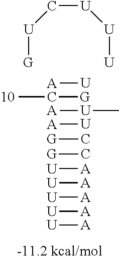 |
| 11982/ | 11 nt PPIB | SS: U.G.U.U.C.C.A.A.A.A.A | | |
| 17 | duplex | AS: U.U.U.U.U.G.G.A.A.C.A | | |
| 18 | Control | | | |
| 11978/ 19 | 11 nt PPIB | U.U.U.U.U.G.G.A.A.C.A.G.U.C.U.U. I.I.I.C.C.A.A.A.A.A | 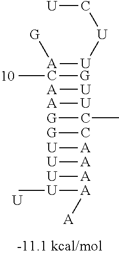 | 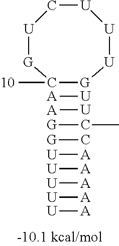 |
| 11983/ | 10 nt PPIB | SS: G.U.U.C.C.A.A.A.A.A | | |
| 20 | duplex | AS: U.U.U.U.U.G.G.A.A.C | | |
| 21 | Control | | | |
| 10460/ | PPIB 25-bp | SS: P.mC.mU.mC.mU.U.C.G.G.A.A.A.G. | | |
| 22 | Duplex | A.C.U.G.U.U.C.C.A.mA.mA.mA.mA | | |
| 23 | | AS: U.U.U.U.U.G.G.A.A.C.A.G.U.C.U.U. | | |
| | | U.C.C.G.A.A.G.A.G | | |
| 11994/ 24 | shRNA Ctrl Sequence 5′->3′ | U.U.U.U.U.G.G.A.A.C.A.G.U.C.U.U. U.C.C.U.U.C.A.A.G.A.G.A.G.G.A.A. A.G.A.C.U.G.U.U.C.C.A.A.A.A.A | 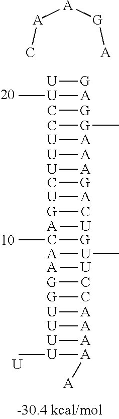 | |
| 11995/ 25 | shRNA Ctrl Sequence2 5′->3′ | U.U.U.U.U.G.G.A.A.C.A.G.U.C.U.U. U.C.C.C.U.U.C.C.G.G.A.A.A.G.A.C. U.G.U.U.C.C.A.A.A.A.A | 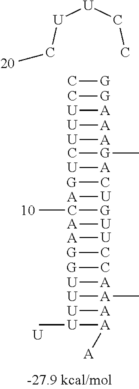 | |
| 12000/ | PPIB Alt | SS: G.G.A.A.A.G.A.C.U.G.U.U.C.C.A.A. | | |
| 26 | Control | A.A.A | | |
| 27 | | AS: U.U.U.U.U.G.G.A.A.C.A.G.U. | | |
| 11989/ 28 | 15 nt MAP4K4 | U.A.G.A.C.U.U.C.C.A.C.A.G.A.A.C. U.C.I.U.U.C.U.G.U.G.G.A.A.G.U.C. U.A | 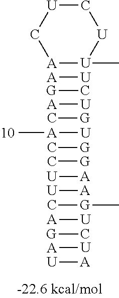 | 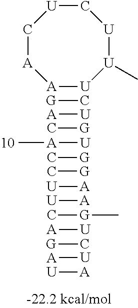 |
| 11964/ | Luc Ctrl | G.U.A.U.C.U.C.U.U.C.A.U.A.G.C.C. | | |
| 29 | 14 nt | U.U.A.A.C.U.A.U.G.A.A.G.A.G.A.U. | | |
| | | A.C | | |
| 11990/ 30 | 13 nt MAP4K4 | U.A.G.A.C.U.U.C.C.A.C.A.G.A.A.C. U.C.I.C.U.G.U.G.G.A.A.G.U.C.U.A | | 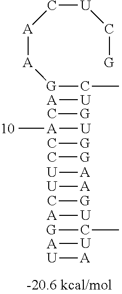 |
| 11965/ | Luc Ctrl | G.U.A.U.C.U.C.U.U.C.A.U.A.G.C.C. | | |
| 31 | 13 nt | U.U.A.U.A.U.G.A.A.G.A.G.A.U.A.C | | |
| 11991/ 32 | 13 nt MAP4K4 | U.A.G.A.C.U.U.C.C.A.C.A.G.A.A.C. U.C.U.G.U.G.G.A.A.G.U.C.U.A | 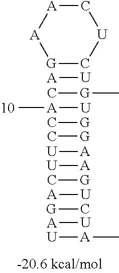 | |
| 11966/ | Luc Ctrl | G.U.A.U.C.U.C.U.U.C.A.U.A.G.C.C. | | |
| 33 | 12 nt | U.U.A.U.G.A.A.G.A.G.A.U.A.C | | |
| 11992/ 34 | 12 nt MAP4K4 | U.A.G.A.C.U.U.C.C.A.C.A.G.A.A.C. U.I.U.G.G.A.A.G.U.C.U.A | 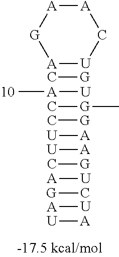 | |
| 11967/ | Luc Ctrl | G.U.A.U.C.U.C.U.U.C.A.U.A.G.C.C. | | |
| 35 | 11 nt | U.U.I.A.A.G.A.G.A.U.A.C | | |
| 11993/ 36 | 10 nt MAP4K4 | U.A.G.A.C.U.U.C.C.A.C.A.G.A.I.I.U. I.I.A.A.G.U.C.U.A | 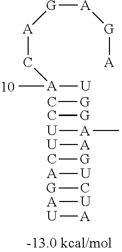 | |
| 11968/ | Luc Ctrl | G.U.A.U.C.U.C.U.U.C.A.U.A.G.C.C. | | |
| 37 | 10 nt | I.I.A.G.A.G.A.U.A.C | | |
| 10461/ | Luc Ctrl 25- | SS: P.mG.mC.mA.mC.U.C.U.G.A.U.U.G. | | |
| 38 | bp Duplex | A.C.A.A.A.U.A.C.G.mA.mU.mU.mU | | |
| 39 | | AS: A.A.A.U.C.G.U.A.U.U.U.G.U.C.A.A. | | |
| | | U.C.A.G.A.G.U.G.C | | |
| 11546/ | MAP4K4 25- | SS: P.mC.mU.mU.mU.G.A.A.G.A.G.U.U. | | |
| 40 | bp Duplex | C.U.G.U.G.G.A.A.G.mU.mC.mU.mA | | |
| 41 | | AS: U.A.G.A.C.U.U.C.C.A.C.A.G.A.A.C. | | |
| | | U.C.U.U.C.A.A.A.G | | |
| 12134/ | 13 nt | U.A.G.A.C.U.U.C.C.A.C.A.G.A.A. | | |
| 42 | MAP4K4 | C.U.C.U.U.G.U.G.G.A.A.G.U.C. | | |
| | | U.A | | |
| 12067/ | 14 nt | U.A.G.A.C.U.U.C.C.A.C.A.G.A.A. | | |
| 43 | MAP4K4 | C.U.C.U.U.C.U.G.U.G.G.A.A.G. | | |
| | | U.C.U.A | | |
| 12069/ | 12 nt | U.A.G.A.C.U.U.C.C.A.C.A.G.A.A. | | |
| 44 | MAP4K4 | C.U.C.U.U.G.U.G.G.A.A.G.U.C. | | |
| | | U.A | | |
| 12071/ | 11 nt | U.A.G.A.C.U.U.C.C.A.C.A.G.A.A. | | |
| 45 | MAP4K4 | C.U.C.U.G.U.G.G.A.A.G.U.C.U. | | |
| | | A | | |
| 12073/ | 10 nt | U.A.G.A.C.U.U.C.C.A.C.A.G.A.A. | | |
| 46 | MAP4K4 | C.U.C.U.U.G.G.A.A.G.U.C.U.A | | |
| 12075/ | 9 nt | U.A.G.A.C.U.U.C.C.A.C.A.G.A.A. | | |
| 47 | MAP4K4 | C.U.C.U.G.G.A.A.G.U.C.U.A | | |
| 12077/ | 8 nt | U.A.G.A.C.U.U.C.C.A.C.A.G.A.A. | | |
| 48 | MAP4K4 | C.U.C.U.G.A.A.G.U.C.U.A | | |
| 12079/ | 7 nt | U.A.G.A.C.U.U.C.C.A.C.A.G.A.A. | | |
| 49 | MAP4K4 | C.U.C.U.A.A.G.U.C.U.A | | |
| 12081/ | 6 nt | U.A.G.A.C.U.U.C.C.A.C.A.G.A.A. | | |
| 50 | MAP4K4 | C.U.C.U.A.G.U.C.U.A | | |
| 12003/ 51 | PPIB Less Active 14 nt | U.U.A.C.A.C.G.A.U.G.G.A.A.U.U.U. G.C.U.U.U.C.C.A.U.C.G.U.G.U.A.A. | 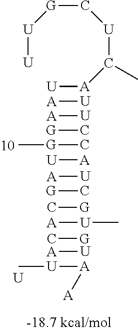 | 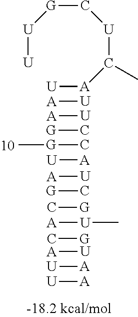 |
| 12004/ 52 | PPIB Less Active 13 nt | U.U.A.C.A.C.G.A.U.G.G.A.A.U.U.U. G.C.U.C.C.A.U.C.G.U.G.U.A.A | 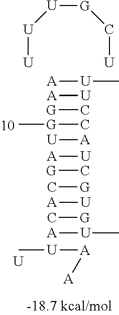 | 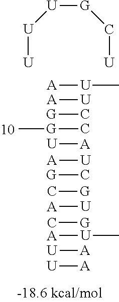 |
| 10463/ | PPIB 25-bp | SS: P.mA.mA.mA.mA.A.C.A.G.C.A.A.A. | | |
| 53 | Duplex (Less | U.U.C.C.A.U.C.G.U.mG.mU.mA.mA | | |
| 54 | active | AS: U.U.A.C.A.C.G.A.U.G.G.A.A.U.U.U. | | |
| | sequence) | G.C.U.G.U.U.U.U.U | | |
| 12034/ 55 | 14 nt SOD1 | U.A.C.U.U.U.C.U.U.C.A.U.U.U.C.C. A.C.C.C.A.A.A.U.G.A.A.G.A.A.A.G. U.A | 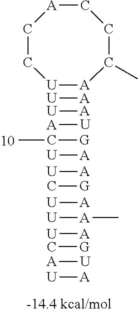 | |
| 12035/ 56 | 13 nt SOD1 | U.A.C.U.U.U.C.U.U.C.A.U.U.U.C.C. A.C.C.A.A.U.G.A.A.G.A.A.A.G.U.A | 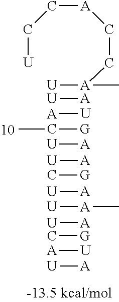 | |
| 12036/ 57 | 12 nt SOD1 | U.A.C.U.U.U.C.U.U.C.A.U.U.U.C.C. A.C.I.U.G.A.A.G.A.A.A.G.U.A | 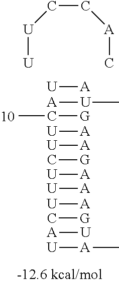 | |
| 12037/ 58 | 12 nt SOD1 | U.A.C.U.U.U.C.U.U.C.A.U.U.U.C.C. A.I.G.A.A.G.A.A.A.G.U.A | 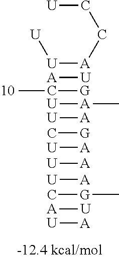 | |
| 12038/ 59 | 10 nt SOD1 | U.A.C.U.U.U.C.U.U.C.A.U.U.U.C.C. I.A.A.G.A.A.A.G.U.A | 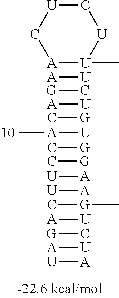 | |
| 12039/ 60 | 9 nt SOD1 | U.A.C.U.U.U.C.U.U.C.A.U.U.U.C.I. A.G.A.A.A.G.U.A | 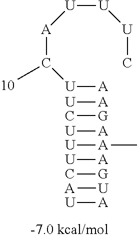 | |
| 12040/ 61 | 8 nt SOD1 | U.A.C.U.U.U.C.U.U.C.A.U.U.U.I.I.A. A.A.G.U.A | 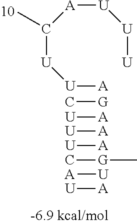 | |
| 12041/ 62 | 7 nt SOD1 | U.A.C.U.U.U.C.U.U.C.A.U.U.I.I.I.A. G.U.A | 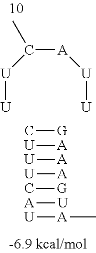 | |
| 10015/ | SOD1 25-bp | SS: P.mG.mG.mC.mA.A.A.G.G.U.G.G.A. | | |
| 63 | Duplex | A.A.U.G.A.A.G.A.A.mA.mG.mU.mA | | |
| 64 | | AS: U.A.C.U.U.U.C.U.U.C.A.U.U.U.C.C. | | |
| | | A.C.C.U.U.U.G.C.C | | |
| 12045/ 65 | SOD1 Targeting 13 nt (based off of 10003) | C.A.A.C.A.U.G.C.C.U.C.U.C.U.U.C. A.U.C.G.A.G.A.G.G.C.A.U.G.U.U.G | 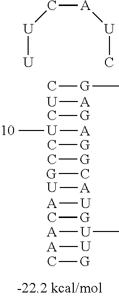 | |
| 10003/ | 25-bp | SS: P.mC.mC.mA.mA.A.G.G.A.U.G.A.A. | | |
| 66 | dsRNA | G.A.G.A.G.G.C.A.U.mG.mU.mU.mG | | |
| 67 | SOD1 | AS: C.A.A.C.A.U.G.C.C.U.C.U.C.U.U.C. | | |
| | Targeting | A.U.C.C.U.U.U.G.G | | |
| 12046/ 68 | SOD1 Targeting 13 nt (based off of 10009) | U.A.A.A.G.U.G.A.G.G.A.C.C.U.G.C. A.C.U.G.G.U.C.C.U.C.A.C.U.U.U.A | 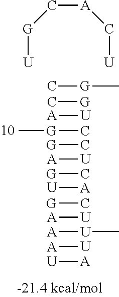 | |
| 10009/ | 25-bp | SS: P.mU.mG.mU.mA.C.C.A.G.U.G.C.A. | | |
| 69 | dsRNA | G.G.U.C.C.U.C.A.C.mU.mU.mU.mA | | |
| 70 | SOD1 | AS: U.A.A.A.G.U.G.A.G.G.A.C.C.U.G.C. | | |
| | Targeting | A.C.U.G.G.U.A.C.A | | |
| 12047/ 71 | SOD1 Targeting 14 nt (based off of 10011) | U.C.A.G.C.A.G.U.C.A.C.A.U.U.G.C. C.C.A.A.U.G.U.G.A.C.U.G.C.U.G.A | 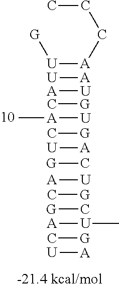 | |
| 10011/ | 25-bp | SS: P.mG.mA.mG.mA.C.U.U.G.G.G.C.A. | | |
| 72 | dsRNA | A.U.G.U.G.A.C.U.G.mC.mU.mG.mA | | |
| 73 | SOD1 | AS: U.C.A.G.C.A.G.U.C.A.C.A.U.U.G.C. | | |
| | Targeting | C.C.A.A.G.U.C.U.C | | |
| 12048/ 74 | SOD1 Targeting 13 nt (based off of 10023) | C.A.G.A.A.U.C.U.U.C.A.A.U.A.G.A. C.A.C.A.U.U.G.A.A.G.A.U.U.C.U.G | 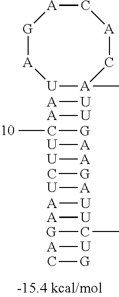 | |
| 10023/ | 25-bp | SS: P.mG.mC.mC.mG.A.U.G.U.G.U.C.U. | | |
| 75 | dsRNA | A.U.U.G.A.A.G.A.U.mU.mC.mU.mG | | |
| 76 | SOD1 | AS: C.A.G.A.A.U.C.U.U.C.A.A.U.A.G.A. | | |
| | Targeting | C.A.C.A.U.C.G.G.C | | |
| 12049/ 77 | SOD1 Targeting 14 nt (based off of 10089) | U.G.U.A.C.U.U.U.C.U.U.C.A.U.U.U. C.C.A.U.G.A.A.G.A.A.A.G.U.A.C.A | 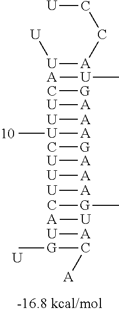 | |
| 10089/ | 25-bp | SS: P.mC.mA.mA.mA.G.G.U.G.G.A.A.A. | | |
| 78 | dsRNA | U.G.A.A.G.A.A.A.G.mU.mA.mC.mA | | |
| 79 | SOD1 | AS: U.G.U.A.C.U.U.U.C.U.U.C.A.U.U.U. | | |
| | Targeting | C.C.A.C.C.U.U.U.G | | |
| 12050/ 82 | SOD1 Targeting 13 nt (based off of 10095) | A.A.C.A.U.G.C.C.U.C.U.C.U.U.C.A. U.C.C.A.G.A.G.A.G.G.C.A.U.G.U.U | 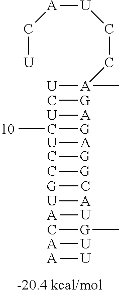 | |
| 10095/ | 25-bp | SS: P.mG.mC.mC.mA.A.A.G.G.A.U.G.A. | | |
| 83 | dsRNA | A.G.A.G.A.G.G.C.A.mU.mG.mU.mU | | |
| 84 | SOD1 | AS: A.A.C.A.U.G.C.C.U.C.U.C.U.U.C.A. | | |
| | Targeting | U.C.C.U.U.U.G.G.C | | |
| 12051/ 85 | SOD1 Targeting 14 nt (based off of 10097) | U.C.C.A.A.C.A.U.G.C.C.U.C.U.C.U. U.C.A.G.A.G.G.C.A.U.G.U.U.G.G.A | 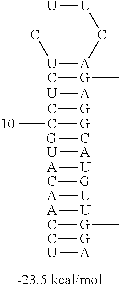 | |
| 10097/ | 25-bp | SS: P.mA.mA.mA.mG.G.A.U.G.A.A.G.A. | | |
| 86 | dsRNA | G.A.G.G.C.A.U.G.U.mU.mG.mG.mA | | |
| 87 | SOD1 | AS: U.C.C.A.A.C.A.U.G.C.C.U.C.U.C.U. | | |
| | Targeting | U.C.A.U.C.C.U.U.U | | |
| 12052/ 88 | SOD1 Targeting 13 nt (based off of 10288) | U.U.C.A.U.U.U.C.C.A.C.C.U.U.U.G. C.C.C.A.G.G.U.G.G.A.A.A.U.G.A.A | 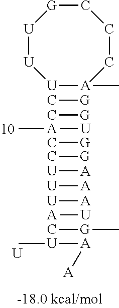 | |
| 10288/ | 25-bp | SS: P.mU.mG.mA.mC.U.U.G.G.G.C.A.A. | | |
| 89 | dsRNA | A.G.G.U.G.G.A.A.A.mU.mG.mA.mA | | |
| 90 | SOD1 | AS: U.U.C.A.U.U.U.C.C.A.C.C.U.U.U.G. | | |
| | Targeting | C.C.C.A.A.G.U.C.A | | |
| 12053/ 91 | SOD1 Targeting 13 nt (based off of 10256) | U.C.U.C.C.A.A.C.A.U.G.C.C.U.C.U. C.U.U.G.G.C.A.U.G.U.U.G.G.A.G.A | 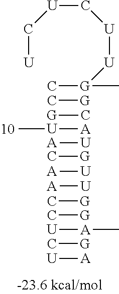 | |
| 10256/ | 25-bp | SS: P.mA.mG.mG.mA.U.G.A.A.G.A.G.A. | | |
| 92 | dsRNA | G.G.C.A.U.G.U.U.G.mG.mA.mG.mA | | |
| 93 | SOD1 | AS: U.C.U.C.C.A.A.C.A.U.G.C.C.U.C.U. | | |
| | Targeting | C.U.U.C.A.U.C.C.U | | |
| 12054/ 94 | SOD1 Targeting 13 nt (based off of 10282) | G.A.U.U.A.A.A.G.U.G.A.G.G.A.C.C. U.G.C.C.C.U.C.A.C.U.U.U.A.A.U.C | 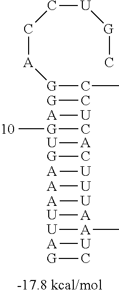 | |
| 10282/ | 25-bp | SS: P.mA.mC.mC.mA.G.U.G.C.A.G.G.U. | | |
| 95 | dsRNA | C.C.U.C.A.C.U.U.U.mA.mA.mU.mC | | |
| 96 | SOD1 | AS: G.A.U.U.A.A.A.G.U.G.A.G.G.A.C.C. | | |
| | Targeting | U.G.C.A.C.U.G.G.U | | |
| 12055/ 97 | SOD1 Targeting 14 nt (based off of 10266) | U.G.G.C.C.C.A.C.C.G.U.G.U.U.U.U. C.U.G.A.C.A.C.G.G.U.G.G.G.C.C.A | 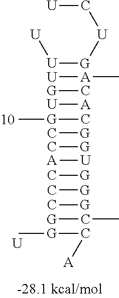 | |
| 10266/ | 25-bp | SS: P.mU.mC.mU.mA.U.C.C.A.G.A.A.A. | | |
| 98 | dsRNA | A.C.A.C.G.G.U.G.G.mG.mC.mC.mA | | |
| 99 | SOD1 | AS: U.G.G.C.C.C.A.C.C.G.U.G.U.U.U.U. | | |
| | Targeting | C.U.G.G.A.U.A.G.A | | |
| 12056/ 100 | SOD1 Targeting 13 nt (based off of 10308) | C.G.A.A.A.U.U.G.A.U.G.A.U.G.C.C. C.U.G.A.U.C.A.U.C.A.A.U.U.U.C.G | 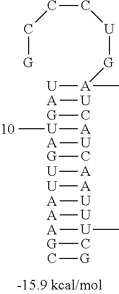 | |
| 10308/ | 25-bp | SS: P.mC.mC.mA.mG.U.G.C.A.G.G.G.C. | | |
| 101 | dsRNA | A.U.C.A.U.C.A.A.U.mU.mU.mC.mG | | |
| 102 | SOD1 | AS: C.G.A.A.A.U.U.G.A.U.G.A.U.G.C.C. | | |
| | Targeting | C.U.G.C.A.C.U.G.G | | |
| 12057/ 103 | SOD1 Targeting 13 nt (based off of 10314) | A.C.A.C.C.U.U.C.A.C.U.G.G.U.C.C. A.U.U.C.C.A.G.U.G.A.A.G.G.U.G.U | 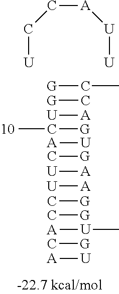 | |
| 10314/ | 25-bp | SS: P.mG.mA.mA.mA.G.U.A.A.U.G.G.A. | | |
| 104 | dsRNA | C.C.A.G.U.G.A.A.G.mG.mU.mG.mU | | |
| 105 | SOD1 | AS: A.C.A.C.C.U.U.C.A.C.U.G.G.U.C.C. | | |
| | Targeting | A.U.U.A.C.U.U.U.C | | |
| 12058/ 106 | SOD1 Targeting 13 nt (based off of 10262) | A.U.C.U.U.C.A.A.U.A.G.A.C.A.C.A. U.C.G.G.U.C.U.A.U.U.G.A.A.G.A.U | 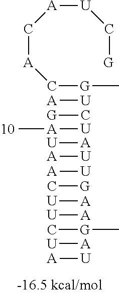 | |
| 10262/ | 25-bp | SS: P.mU.mG.mU.mG.G.C.C.G.A.U.G.U. | | |
| 107 | dsRNA | G.U.C.U.A.U.U.G.A.mA.mG.mA.mU | | |
| 108 | SOD1 | AS: A.U.C.U.U.C.A.A.U.A.G.A.C.A.C.A. | | |
| | Targeting | U.C.G.G.C.C.A.C.A | | |
| 12059/ 109 | SOD1 Targeting 13 nt (based off of 10265) | U.U.U.G.U.C.A.G.C.A.G.U.C.A.C.A. U.U.G.G.A.C.U.G.C.U.G.A.C.A.A.A | 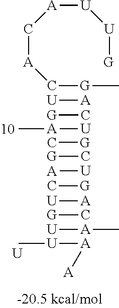 | |
| 10265/ | 25-bp | SS: P.mC.mU.mU.mG.G.G.C.A.A.U.G.U. | | |
| 110 | dsRNA | G.A.C.U.G.C.U.G.A.mC.mA.mA.mA | | |
| | SOD1 | AS: U.U.U.G.U.C.A.G.C.A.G.U.C.A.C.A. | | |
| | Targeting | U.U.G.C.C.C.A.A.G | | |

Key:
P 5′ Phosphate
I 2-deoxy inosine (shading on structure indicates inosine)
m 2′-O Methyl Base Modification
. Normal RNA backbone linkage
G Guanosine
U Uridine
A Adenosine
C Cytidine
SS: Sense (passenger) Strand (duplexes only)
AS: Antisense (guide) Strand (duplexes only)

Data in FIG. 9 showed that, in this example, constructs with at least a 12-bp stem region were effective at all concentrations tested (10 nM, 5 nM, and 1 nM), while constructs with 10-bp or 11-bp were not effective.

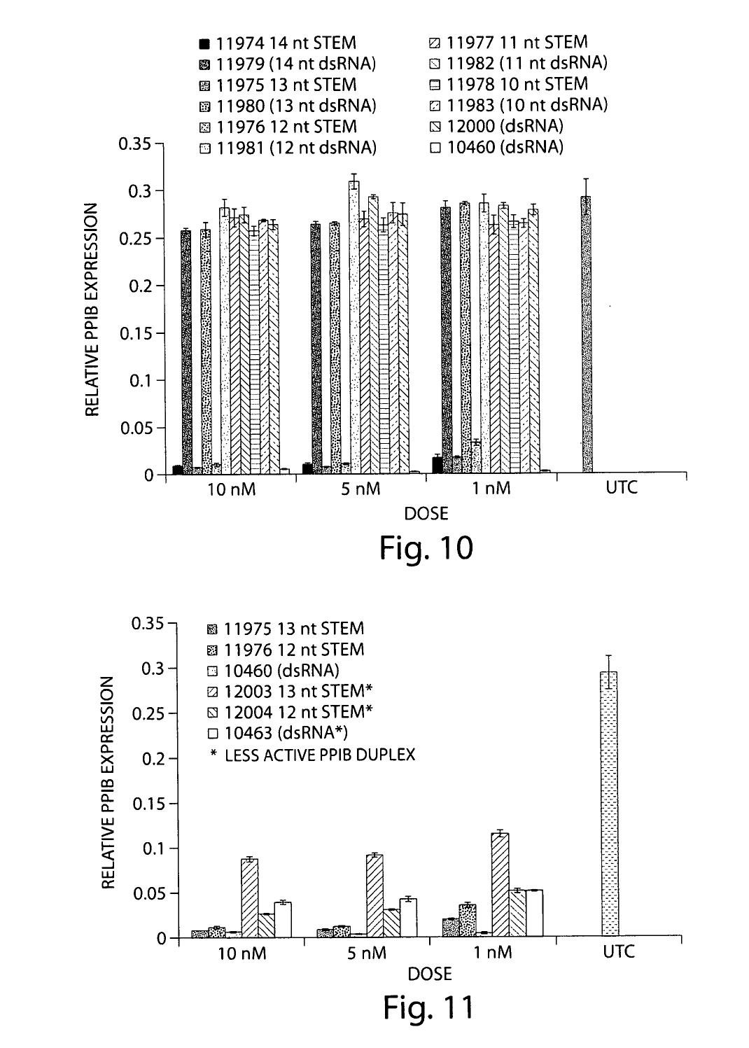

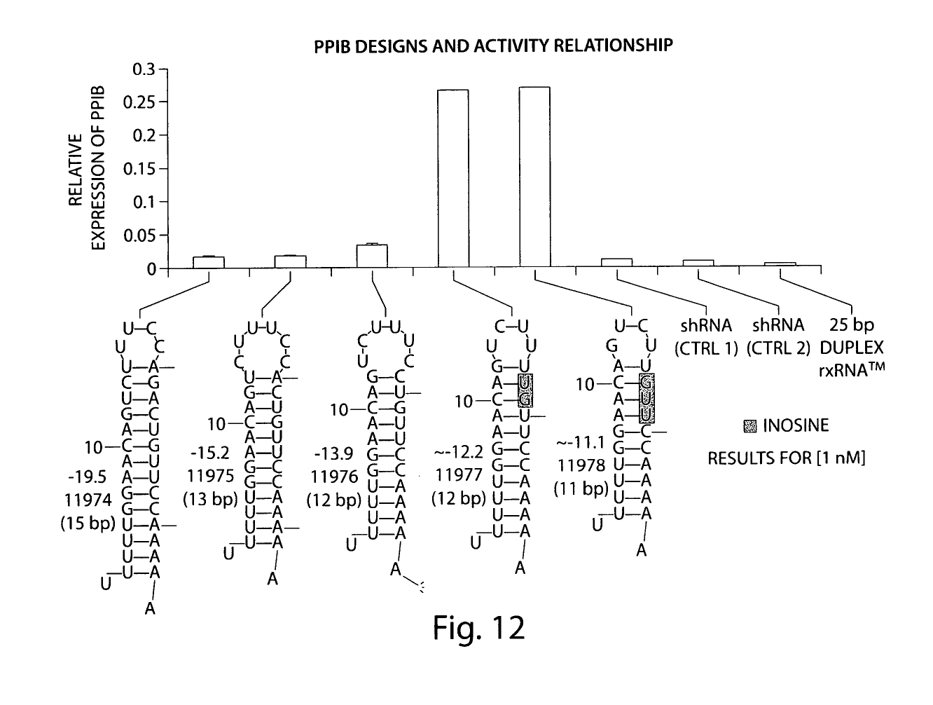

Similar experiments were repeated with the inclusion of matching negative controls (for example, 11979 is a matching negative control for 11974, etc.). Sequence information for the negative controls is also included in the table above. For the negative controls, sequences for only one of the two strands were listed. Oligo 12000 is an alternative negative control for PPIB. The results were shown in FIG. 10.

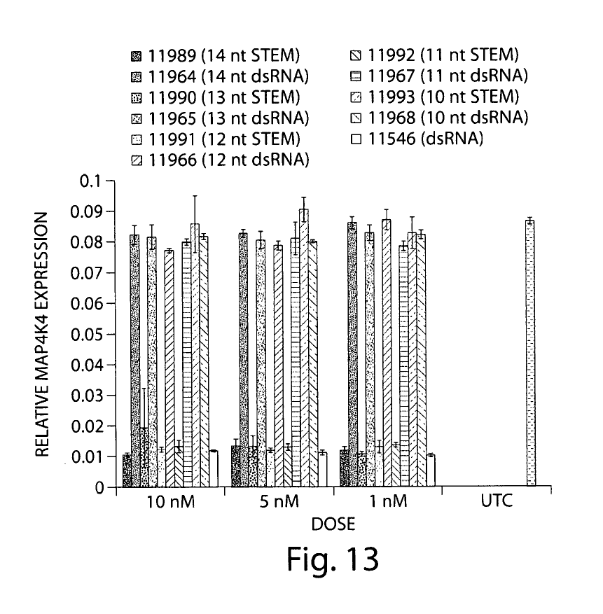

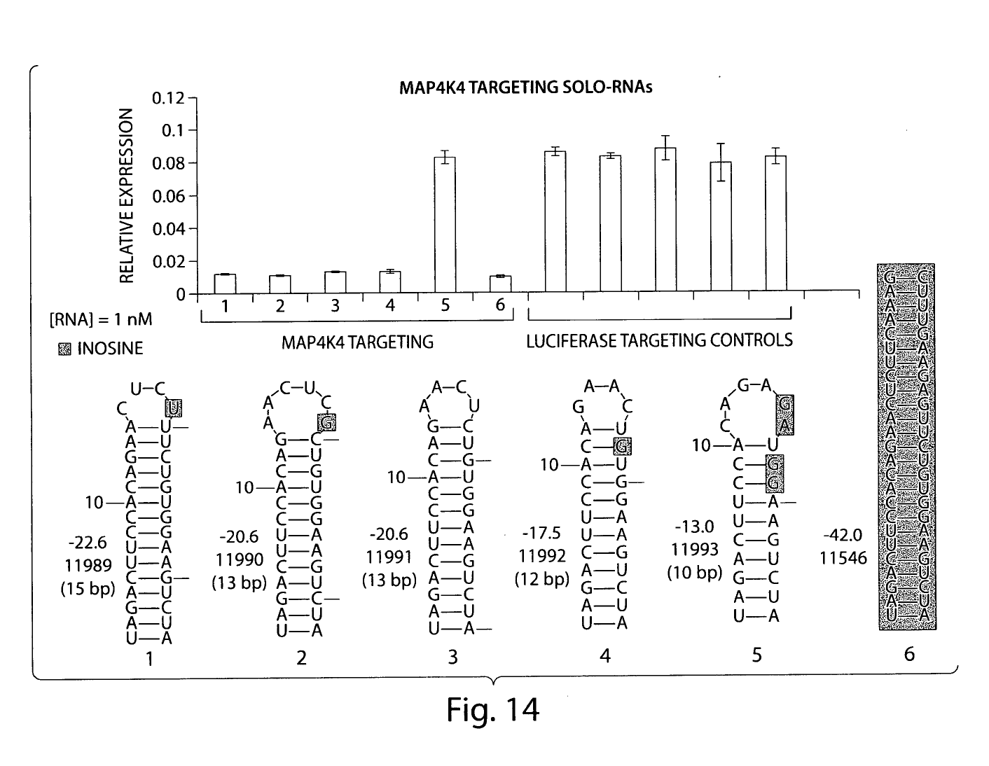

In another similar experiment, MAP4K4 (MAP Kinase Kinase Kinase Kinase 4) was used as a target gene, and constructs having 10-14 bp stem regions were tested. In this experiment, as shown in FIG. 13 constructs having at least 11 bp stems were all about equally effective, although constructs having 10 bp stems were ineffective (see also 10 bp structure 11993 in FIG. 14). Matching negative controls were included in this set of experiments, and the single-stranded polynucleotides used for preparing the constructs are listed in the table above.

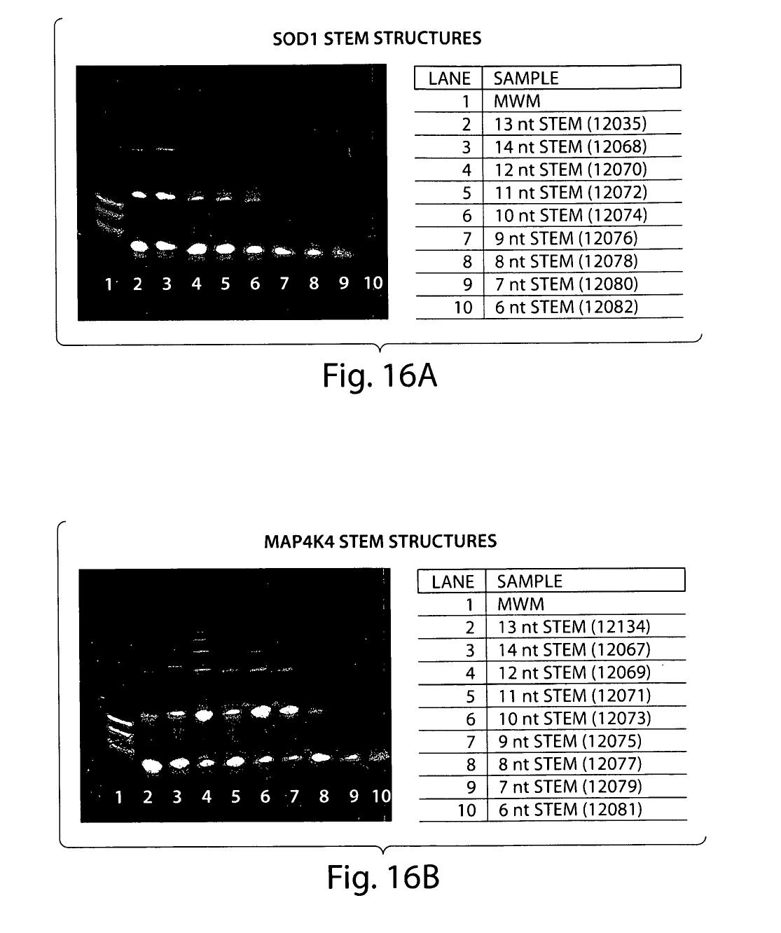

FIG. 16B shows gel images of several MAP4K4-targeting constructs having both monomer and dimer conformations, as well as several other MAP4K4-targeting constructs having only monomer (single-stranded hairpin) conformation. It appears that at least 8 bp is required for dimer formation for this sequence.

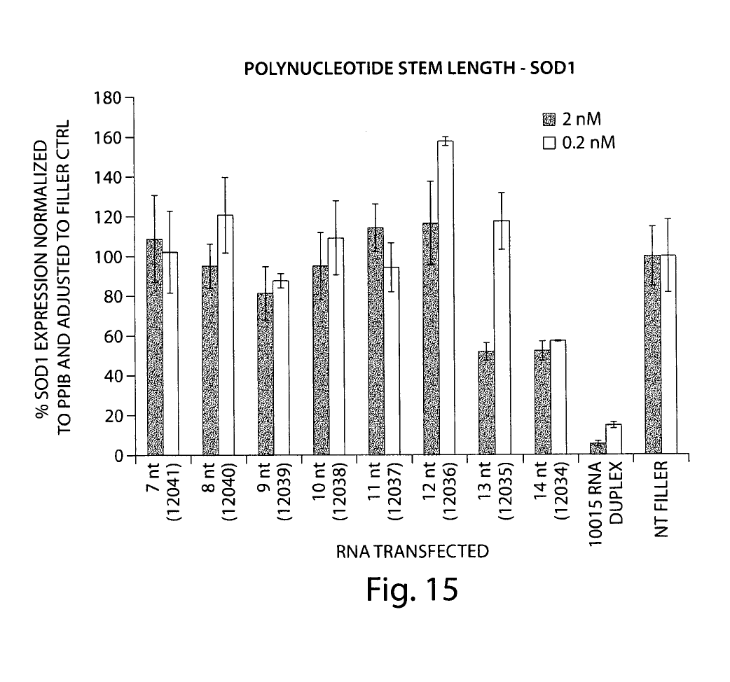

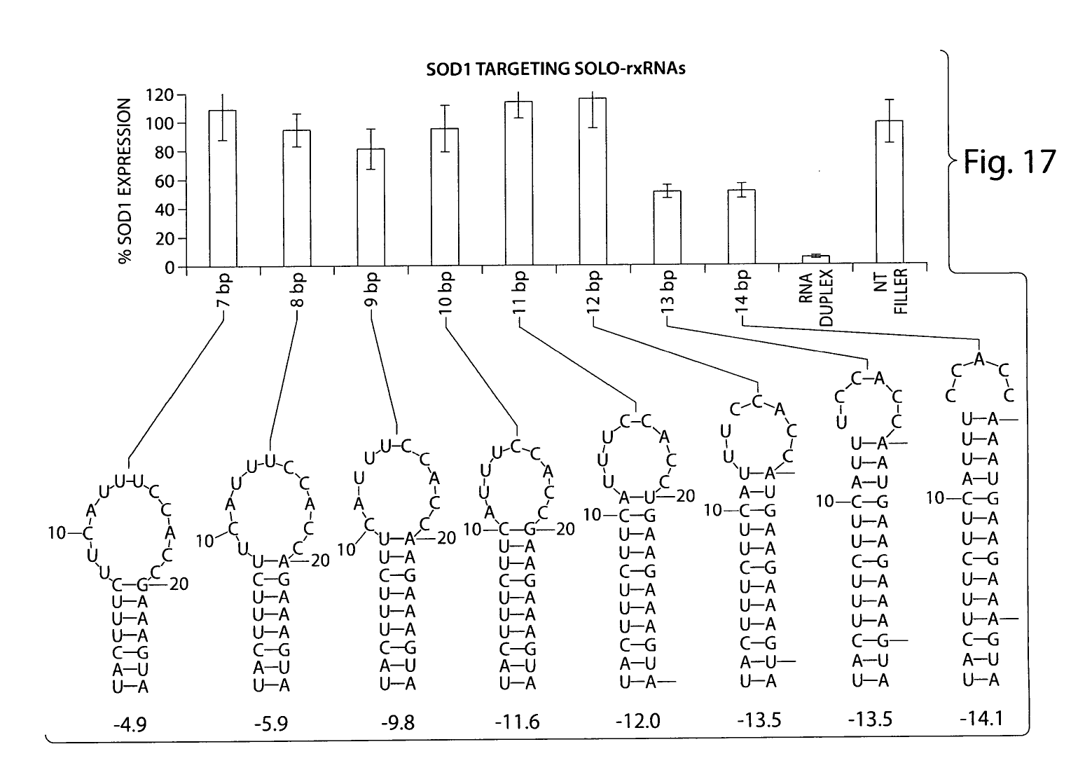

In yet another similar experiment, SOD1 (SuperOxide Dismutase 1) was used as a target gene, and constructs having 7-14 bp stem regions were tested. In this experiment, as shown in FIG. 15, constructs having at least 13 bp stems were about equally effective, in a concentration dependent manner. Matching positive and negative controls were included in this set of experiments, and some of the polynucleotides used are listed in the table above. Similar results are shown in FIG. 17. FIG. 16A shows dimer vs. monomer formation for some exemplary SOD1 constructs having 6-14 bp stem regions. The dimer formation can be detected in stem lengths as low as 9 bp (lane 7).

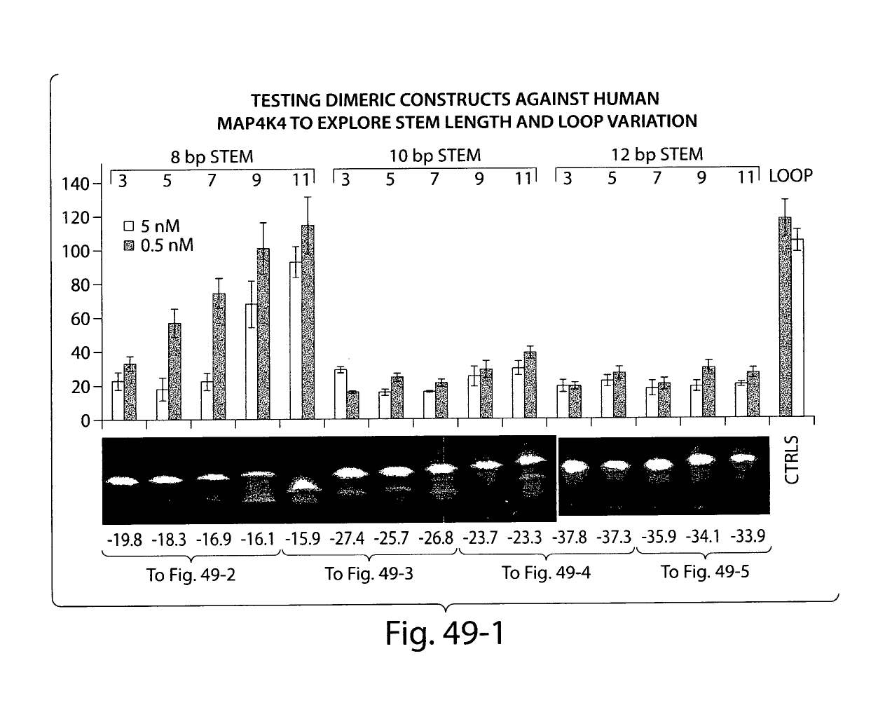

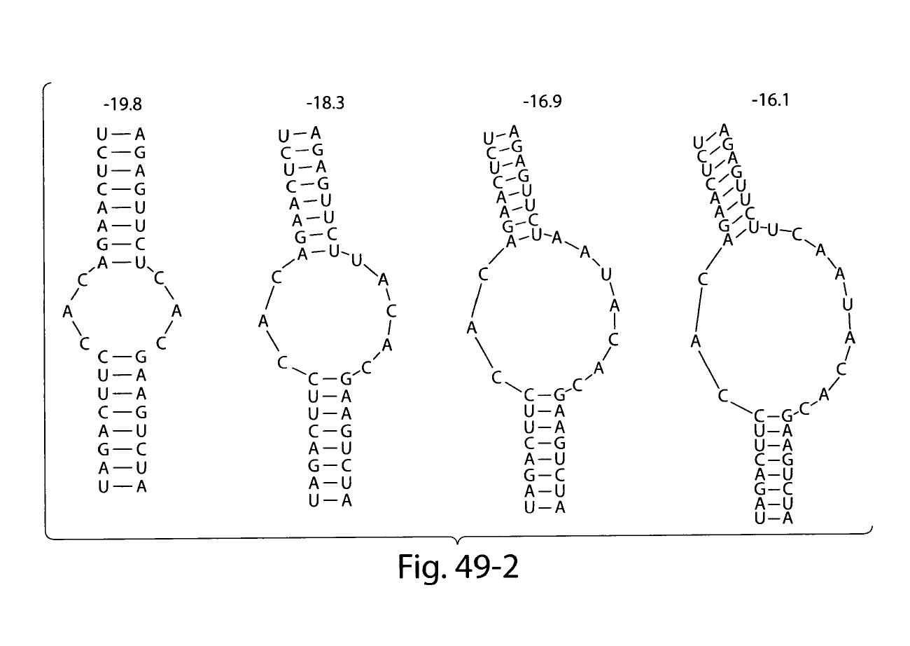

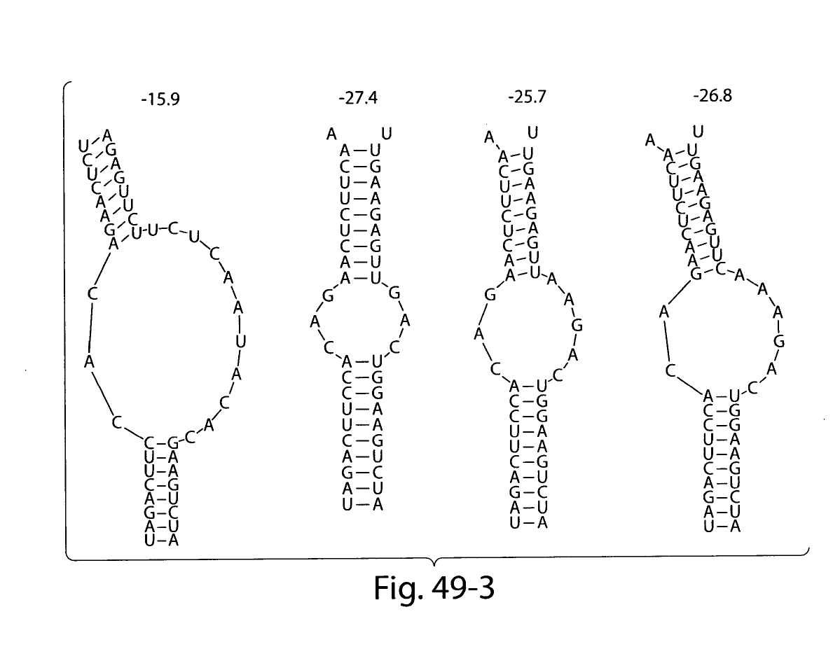

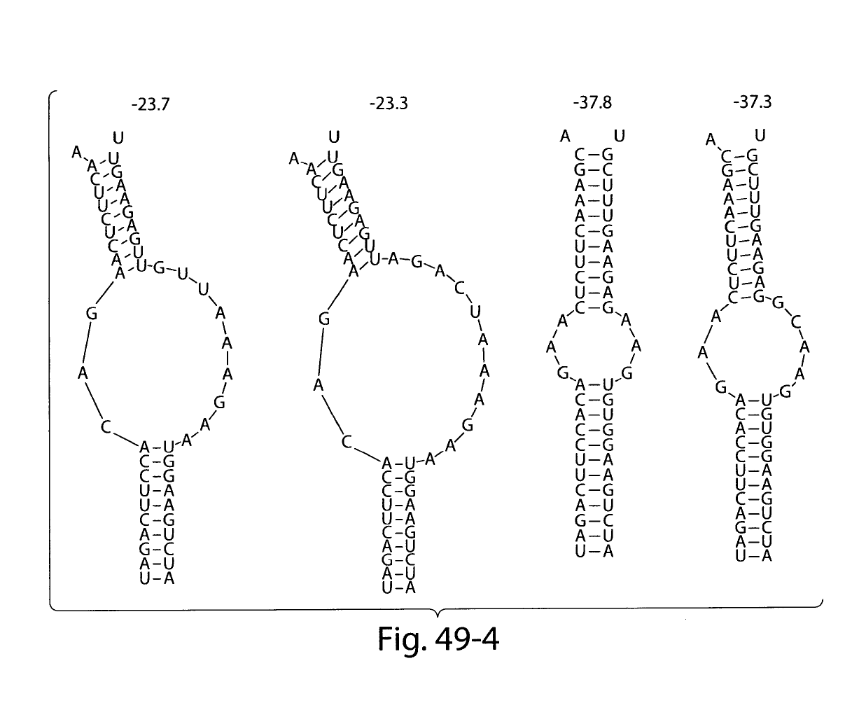

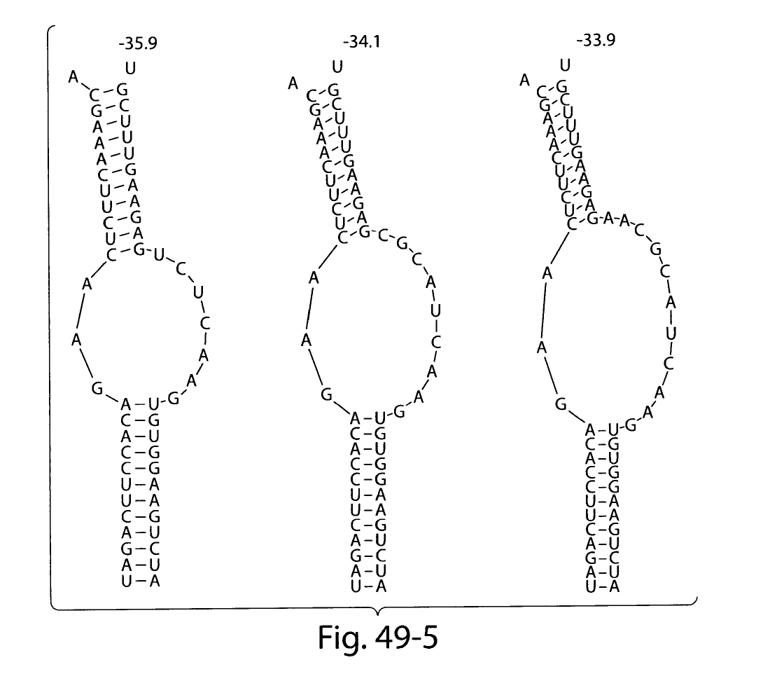

Data in these experiments demonstrate that the subject polynucleotide constructs are effective in initiating RNAi against target genes, so long as the minimal stem region is at least 11 bp in length, preferably at least 12 bp in length. However, the data do not exclude the possibility that a polynucleotide construct with a stem region of less than 11 bp may also be effective against certain target genes. For example, FIG. 49 demonstrates the silencing activity of constructs having 8 bp in the stem region when the loop length is varied to optimize activity. This demonstrates that the subject constructs can have significant gene silencing activity with a minimal stem length of about 8 nucleotides (which may include one or more modifications).

### Example V

### Gene Silencing is Sequence Specific

To determine whether gene silencing through the subject constructs is sequence specific (rather than through some other unillustrated mechanisms), several different constructs targeting the same PPIB gene were used in the transfection assay as described in Example I above. The singles-stranded polynucleotide sequences used for preparing the constructs are listed in the table above.

Data showed that RNAi mediated by the subject constructs is sequence specific. At a less active site on the target gene PPIB, both the more traditional dsRNA construct and the subject constructs were less effective (compare 11975/11976 with 12003/12004).

### Example VI

### Gene Silencing Using Exemplary Constructs—Stem Length Variation

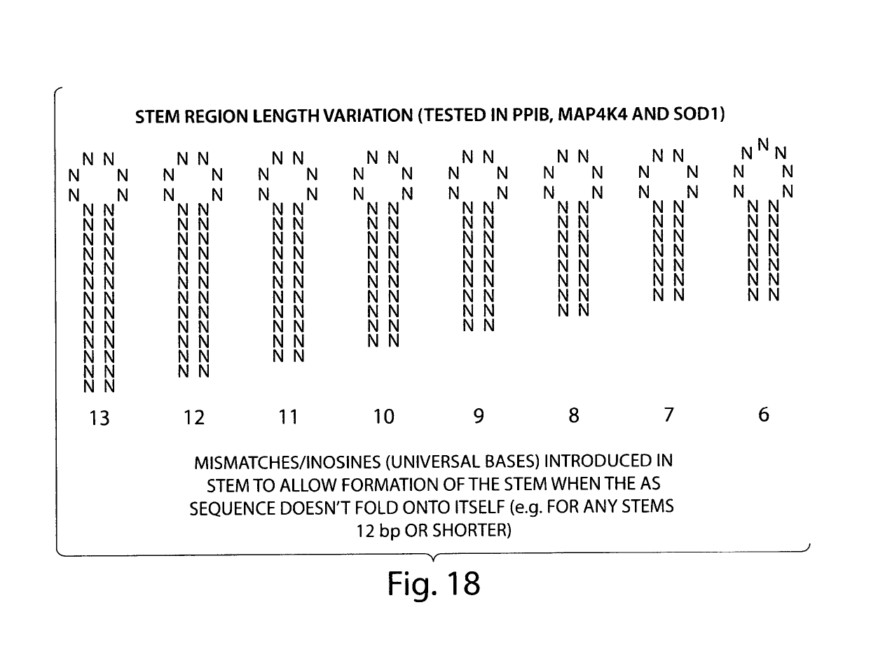

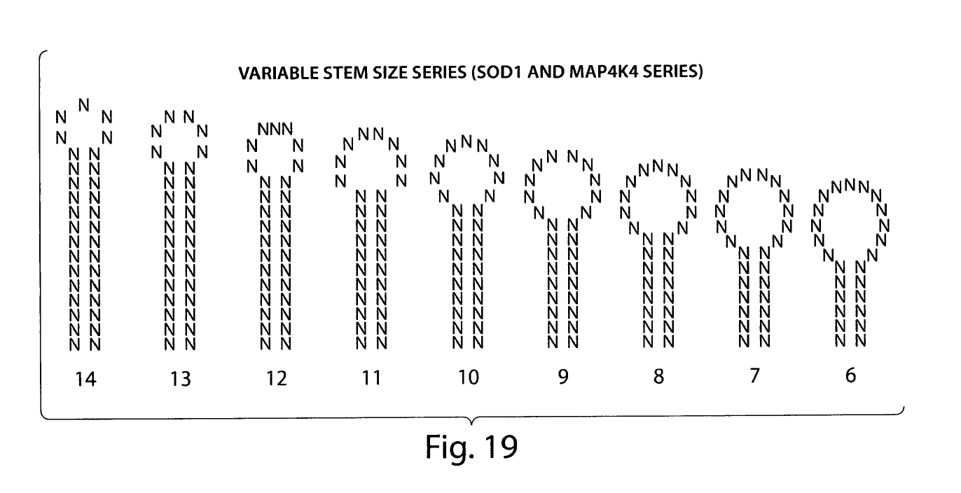

To determine whether gene silencing through the subject constructs depends on the length of the stem region, a series of constructs targeting the same region of the target sequence (e.g., PPIB, MAP4K4, and SOD1, etc.) but having different stem lengths were synthesized and tested using the methods above. See FIG. 18 for sequences of the exemplary tested constructs. Note that, in general, mismatches/inosines (universal bases) may be introduced in the stem to allow formation of the stem when stable base pairing cannot be achieved using the canonical Watson-Crick base pairing. In certain embodiments, such mismatches/inosines (universal bases) were used for any constructs with stems 12 bp or shorter. Also note that in FIG. 18, the loop region was kept constant at 6 bases for each strand, although in other embodiments, other lengths may be used, such as 4, 5, 7, 8, 8, 9, 10 or more bases for each strand, etc. See, for example, some exemplary variations in FIG. 19, showing different loop sizes in combination with different stem lengths.

Data shows that: of all constructs made by converting from a known active dsRNA duplex, stem lengths of 13 bp and 14 bp have worked each time. Furthermore, in an example described in more details below (Example VII), a series of about 15 dsRNA duplexes were converted into their 13-bp-stem counterparts, and the activities of the resulting constructs generally correlate to those of the longer dsRNA. In some instances, RNAi can be consistently achieved using constructs with a stem region of at least about 8 or 9 bp, preferably 11, 12 or more bp.

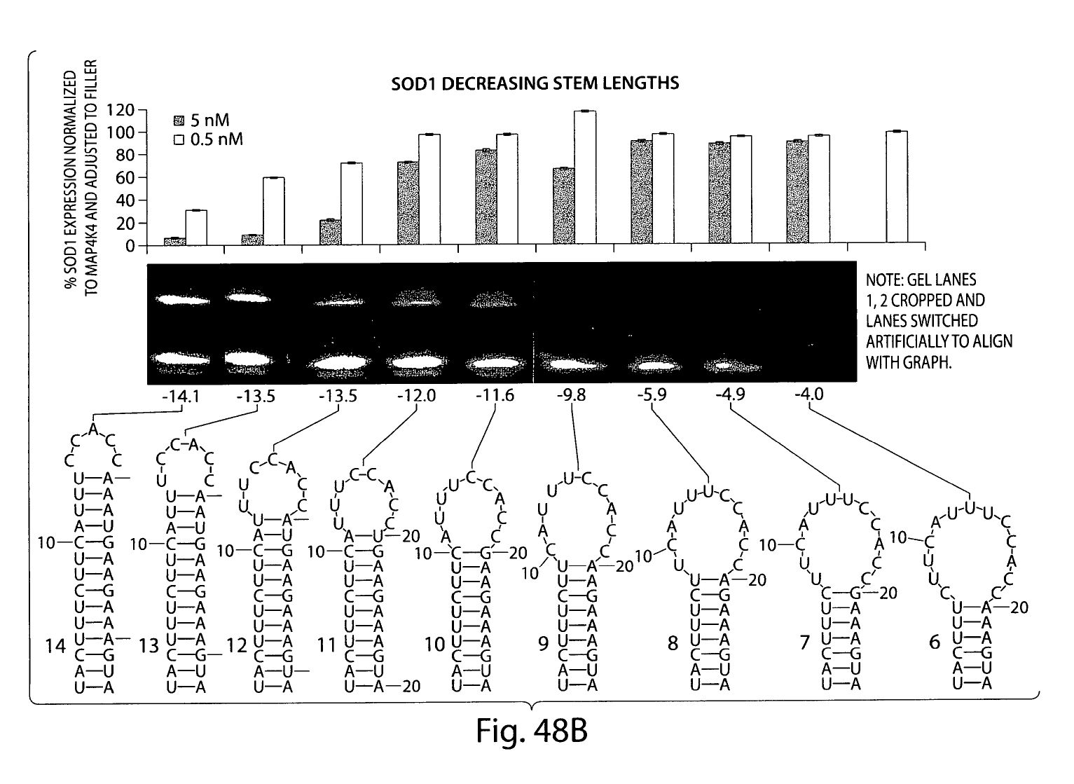

The activity of several specific sequences against MAP4K4 and SOD1 having variable stem lengths and loop sizes were also examined. FIG. 48A shows that stem regions ranging from 10-15 bp can effectively silence MAP4K4 expression. Additionally, FIG. 48A also shows that this activity correlates with efficient dimer formation. The sequence having 8 bp in its stem region forms some dimer, and exhibits some silencing activity. FIG. 48B shows a similar experiment using constructs against SOD1. Similarly, dimer formation can be readily detected in constructs having 10-14 bp in their stem regions. Although not readily visible in FIG. 48B, dimer formation was also faintly detected in the construct having 9 bp in its stem region, and its silencing activity is comparable to the construct having a 11 bp stem region.

### Example VII

### Correlation of Gene Silencing Activities Between the Subject Constructs and Modified dsRNA

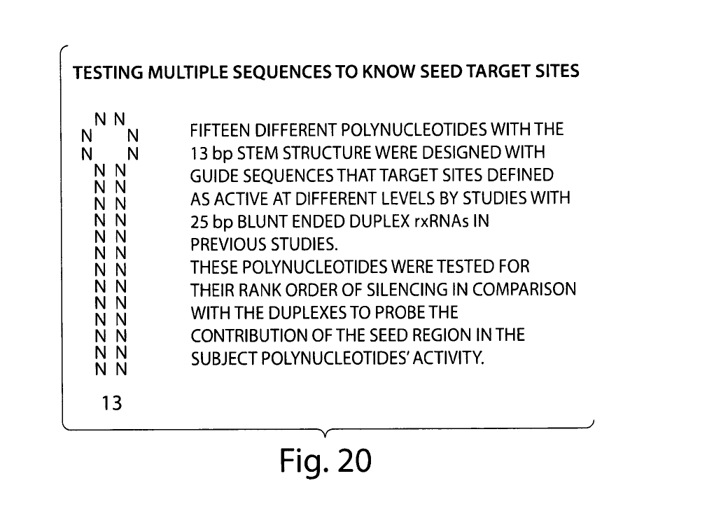

About 15-16 different constructs with fixed 13-bp stem regions were designed, with guide sequences that target sites defined as active at different levels by prior studies with corresponding 25-bp blunt ended dsRNA constructs (e.g., those with 2′-OMe modifications at both ends of the sense strand). See, for example, FIG. 20.

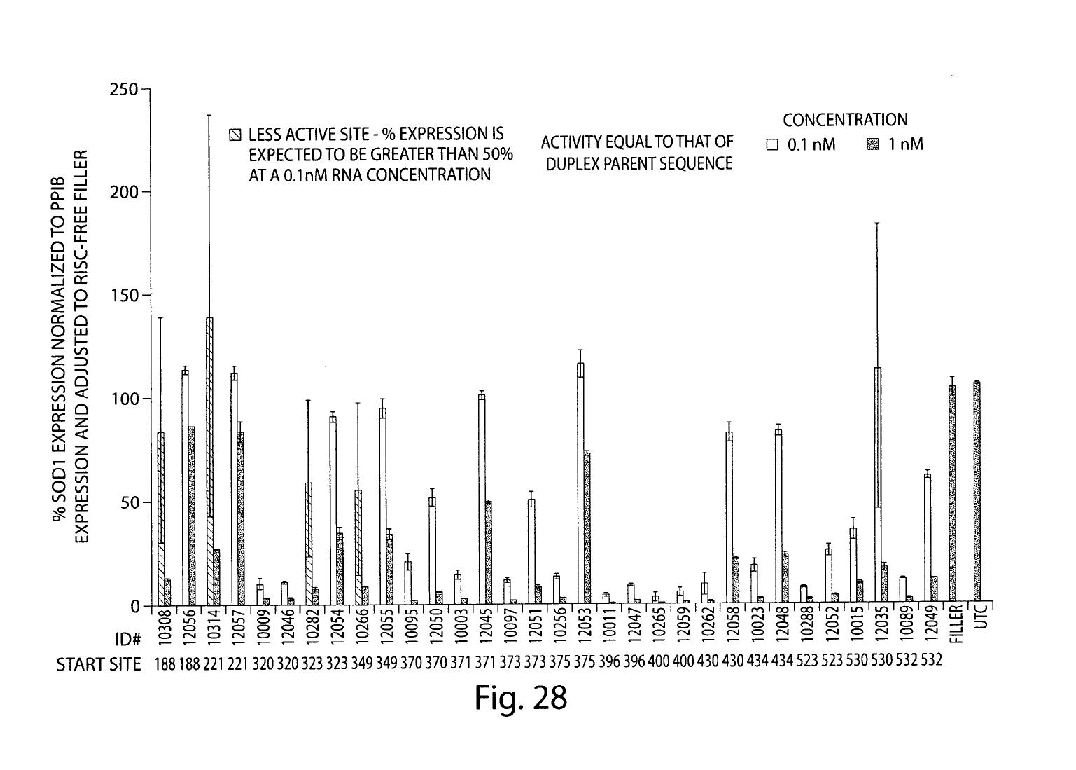

Preliminary data shows that there is a strong correlation between each construct tested and their corresponding dsRNA constructs. For example, FIG. 28 shows 16 sites tested along target gene SOD1. The sites are represented by start site positions on the gene. The pair of doses alternates from dsRNA to the subject constructs for the same site. Of the 16 sites tested, 8 of the subject constructs had activity comparable to their respective parent sequences targeting the same sites. Note that in some of these cases, the parents have no activity and in some other cases, the parents have high activity in silencing the target gene. The remaining 8 subject constructs had activities not as high as their respective parent duplexes targeting the same sites.

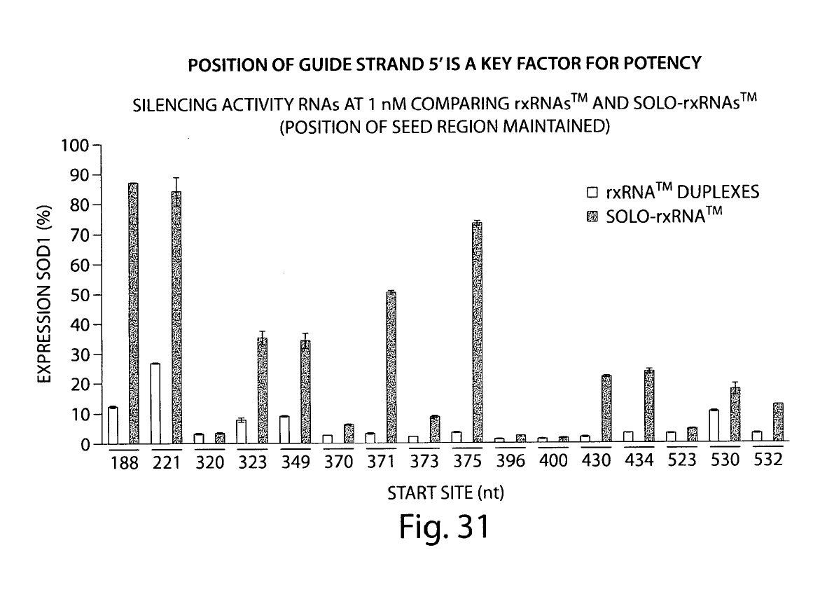

Similar results are shown in FIG. 31, in which the same start sites were examined along target gene SOD1. Here, a direct activity comparison is shown between the solo-rxRNAs and corresponding rxRNA duplex constructs. While not wishing to be bound by any particular theory, it is possible that many factors, such as the free energy of the subject constructs, the ability of the subject constructs to unwind, and the possible target location on the mRNA transcript, may ultimately affect the activity of the subject constructs.

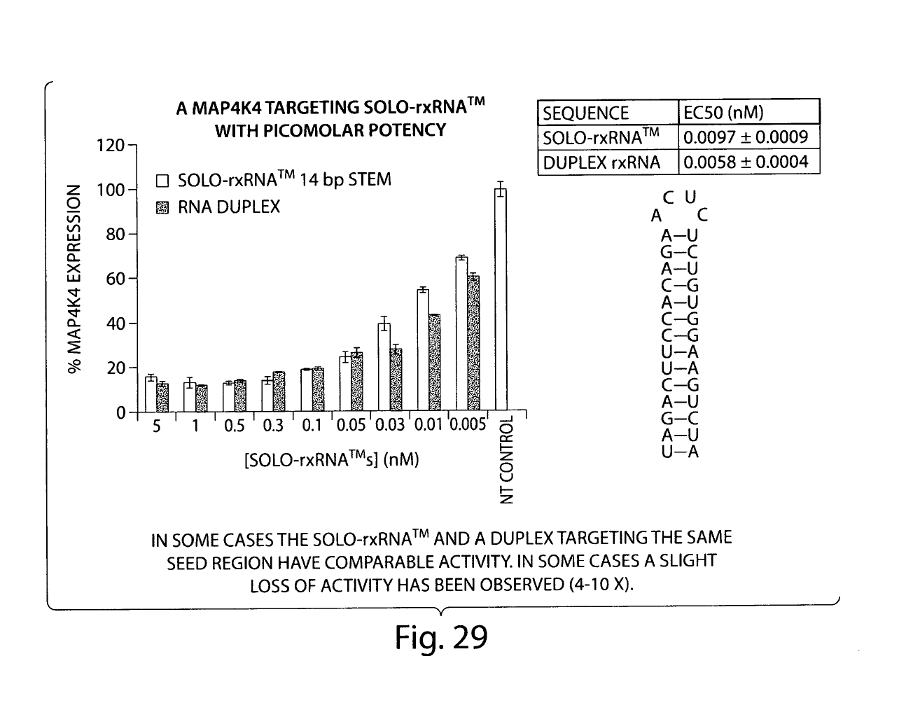

In FIG. 29, a specific solo-rxRNA targeting MAP4K4 and its corresponding rxRNA duplex construct were compared over a wide range of concentrations, and EC₅₀ values for both constructs were determined. The results show that the solo-rxRNA construct and the corresponding rxRNA duplex targeting the same seed region have comparable RNAi activity, both with EC₅₀ values in the picomolar range.

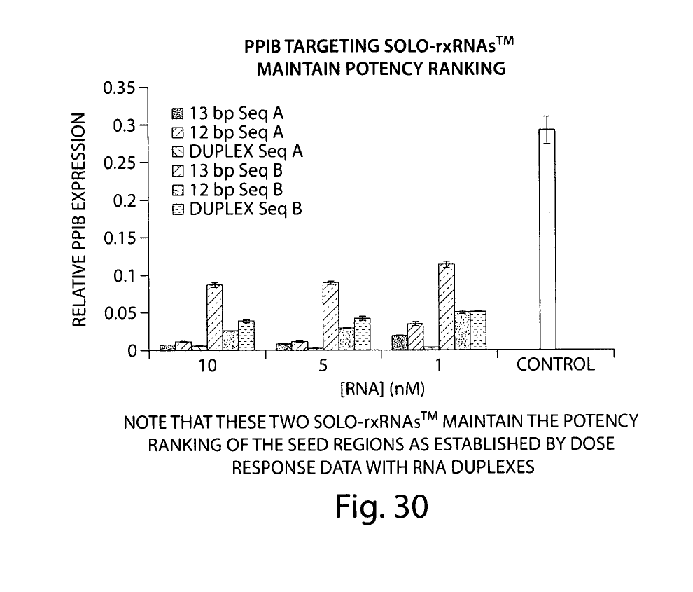

Similar results for two sets of PPIB-targeting solo-rRNA constructs were shown in FIG. 30. Here, “Duplex Sequence A” is a more potent rxRNA compared to “Duplex Sequence B.” Thus, the two solo-rxRNA constructs based on Duplex Sequence A (“13 bp Seq A” and “12 bp Seq A”) are more potent compared to the two solo-rxRNA constructs based on Duplex Sequence B (“13 bp Seq B” and “12 bp Seq B”).

Furthermore, modifying the 2nd nucleotide on the 5′-end with 2′-OMe abolished gene silencing activity of the subject constructs. These data strongly suggests that the subject constructs mediate gene silencing through RNAi pathway rather than through the conventional antisense mechanism, despite the fact that they are not Dicer substrates.

### Example VIII

### Comparison of Gene Silencing Activities

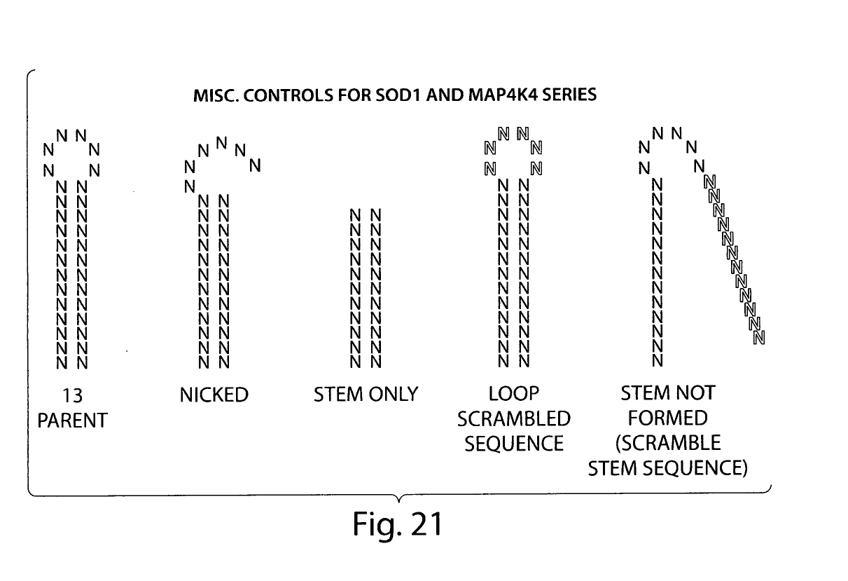

This experiment compares the gene silencing activity of the subject constructs with that of a number of control constructs. The exemplary controls tested are listed in FIG. 21.

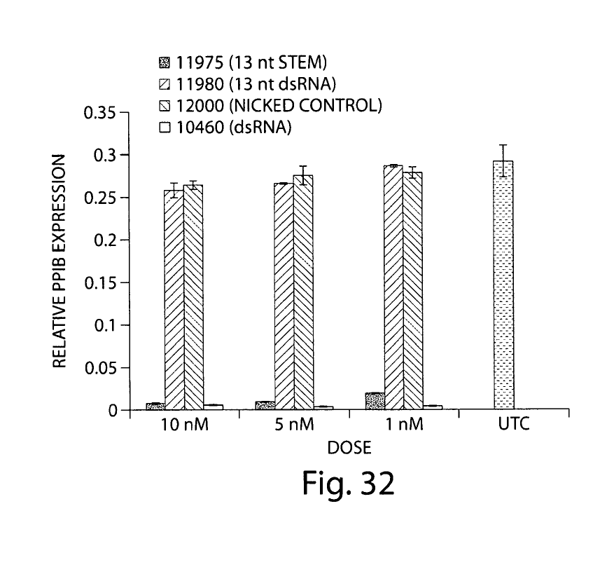

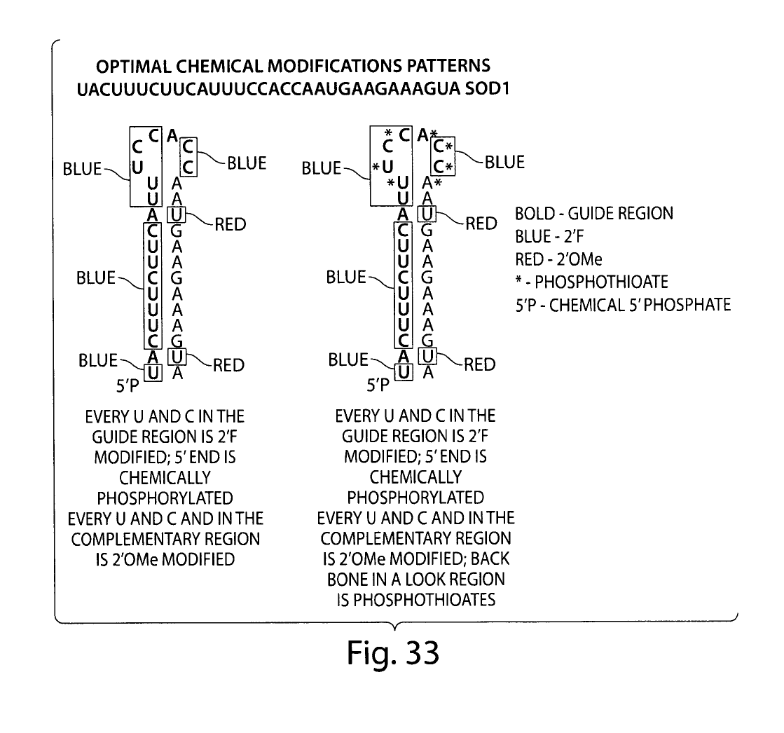

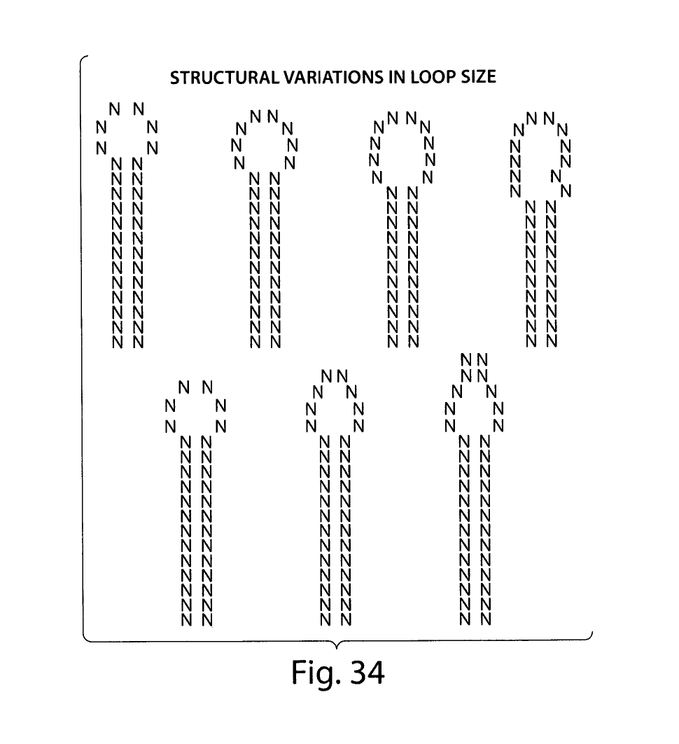

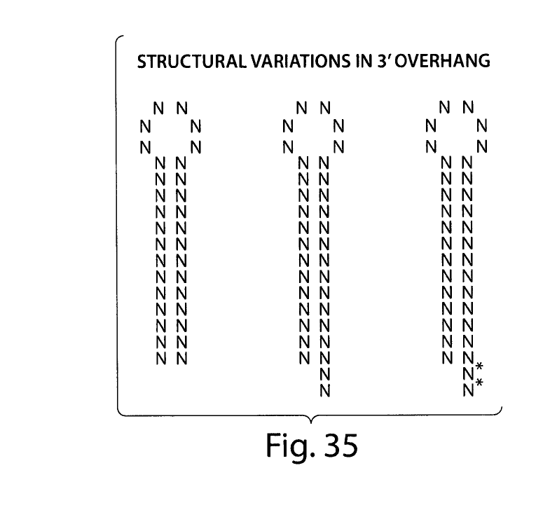

In one embodiment, a nick was created in the loop region, such that the resulting control construct essentially becomes a double-stranded construct with overhangs. For example, if the nick is at the junction of the single and double stranded regions, the control double-stranded construct will have an overhang on the sense or antisense (guide) strand. Otherwise, both the sense and the antisense strands will have an overhang. The nicked constructs may or may not have the same length in the stem region (shown in FIG. 21 is a 12 bp stem region, 1 bp shorter than the parent construct). Preliminary data indicates that the subject double-stranded polynucleotide constructs are much more active than the nicked constructs. In FIG. 32, a control nick construct 12000 has a antisense (guide) sequence of 13 nucleotides, and a sense sequence of 19 nucleotides. This control is essentially inactive compared to the positive control 10460, and the second negative control 11980 (13 bp dsRNA).

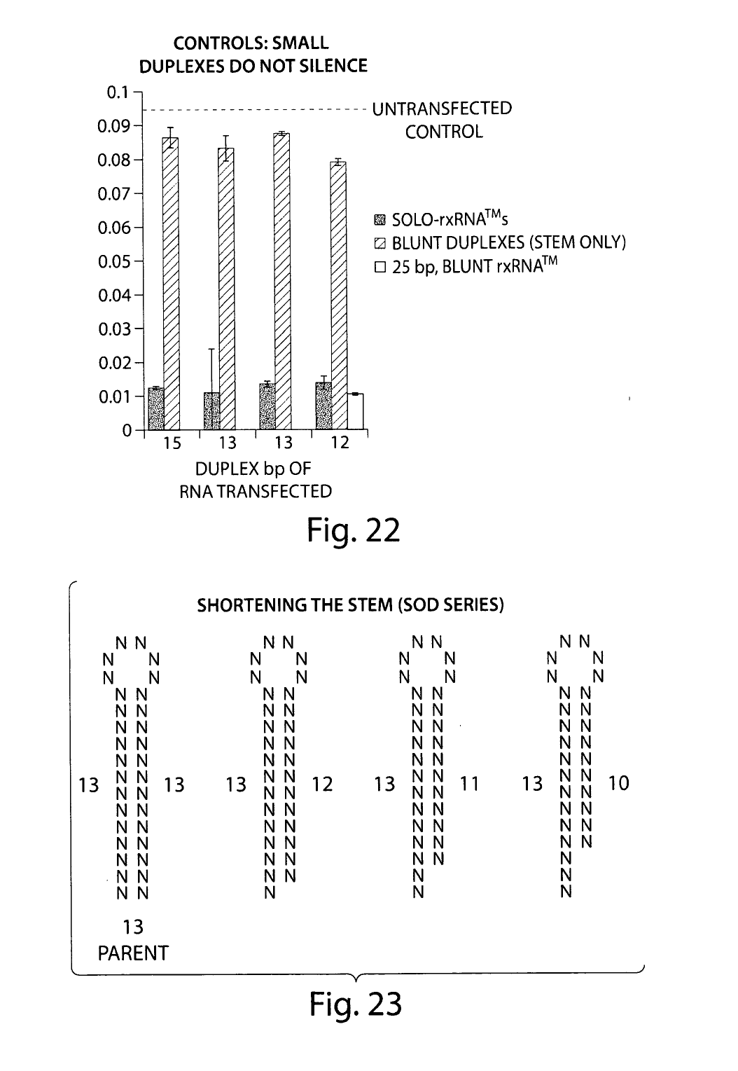

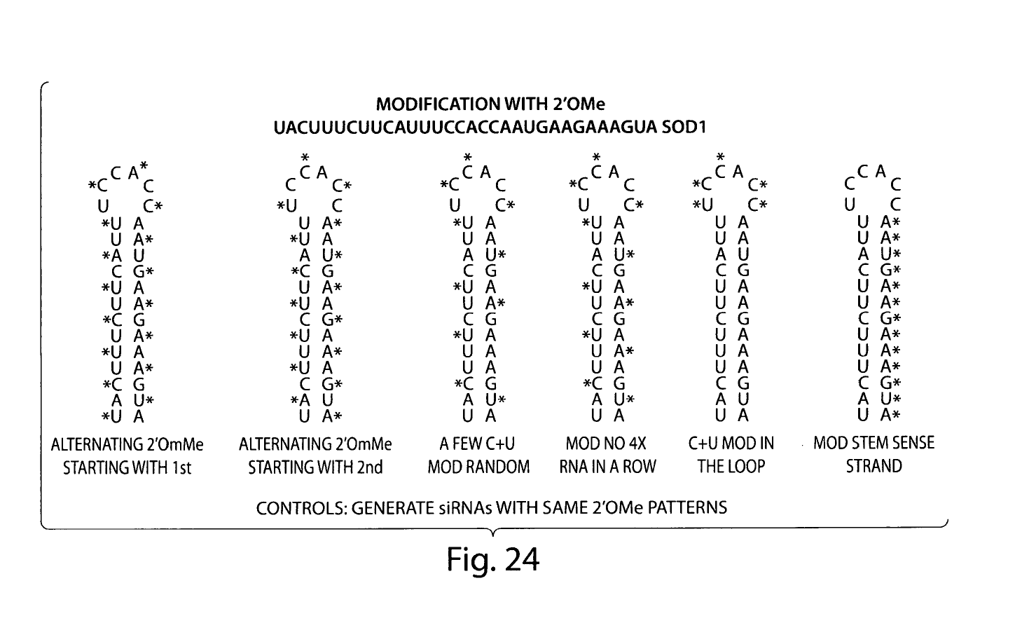

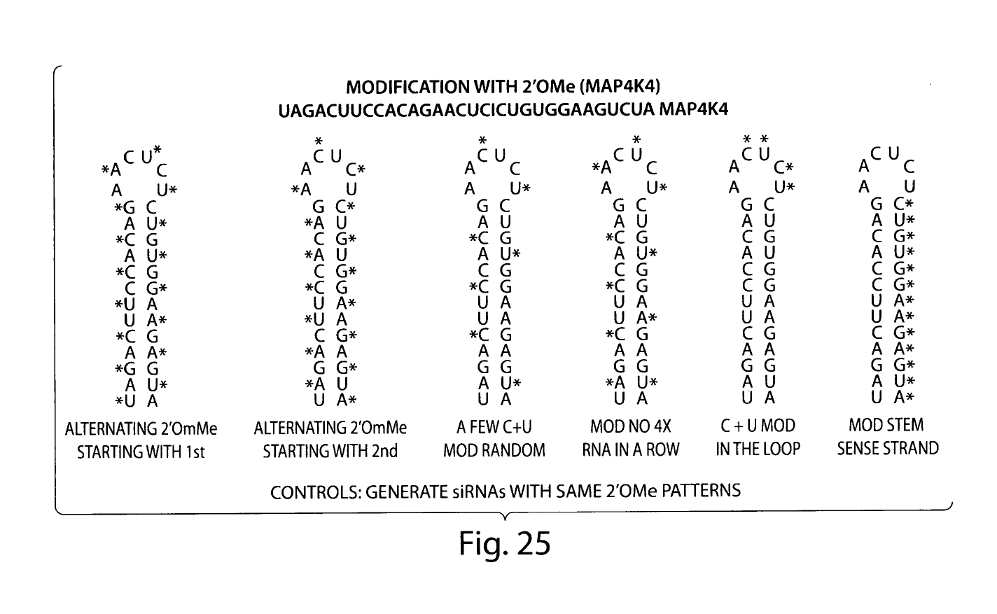

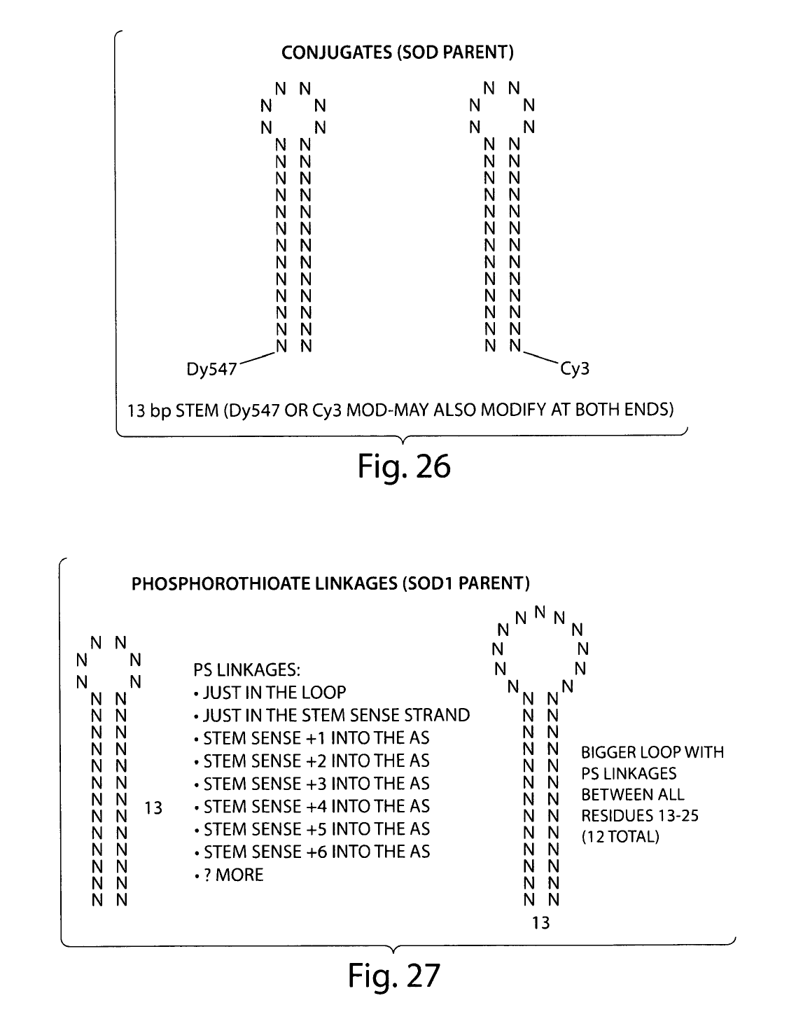

In another embodiment, the single-stranded loop region is completely removed, such that the control construct corresponds to a short double-stranded RNA with blunt ends. No activity was observed for the short dsRNA control. For example, FIG. 22 shows that the stem only sequence shows no silencing activity. However, one 16 bp duplex with 2 nucleotide 3′ end overhangs showed a reduction in activity compared to a corresponding blunt ended 25-bp duplex.

In another embodiment, the loop region is scrambled, such that at least a portion of the guide sequence (at the most 3′-end) does not match the target sequence. In many cases, activity is reduced or even abolished.

In another embodiment, part or whole of the sense sequence is scrambled, such that the stem region on the parent construct is disrupted, and the entire construct becomes a single-stranded sequence. Such constructs are not expected to work through the RNAi pathway, and thus no RNAi activity is expected for these control constructs.

In other embodiments, one or more (e.g., 1, 2, 3, 4, 5, etc.) bases are deleted from one end of the sense strand, such that the most 5′-end of the guide sequence (antisense strand) becomes overhang (i.e., not base-paired). See, for example, FIG. 23. It is expected that progressive deletion of the sense sequence will lead to diminished RNAi activity.

### Example IX

### Modified Constructs

The subject constructs may be modified at one or more nucleotides or phosphodiester linkages to improve one or more biological properties, such as enhanced ability to bind serum albumin, enhanced cellular uptake, increased serum stability and bioavailability, increased construct flexibility and stability, etc.

In certain embodiments, the subject constructs contain one or more 2′-modifications, such as 2′-O-methyl modification. FIGS. 18 and 19 show several exemplary 2′-O-Me modifications in the subject constructs (only the hairpin forms are shown for convenience, the double-stranded stems and loop structure is not shown).

In certain embodiments, the 2′-modification (such as the 2′-O-Me modification) occurs at alternative nucleotides, starting from either the 1st or the 2nd nucleotide from the 5′-end. In certain embodiments, the 2′-modification (such as the 2′-O-Me modification) occurs at a few randomly selected or all pyrimidine nucleotides (C or U). Preferably, the modified nucleotides are roughly evenly distributed along the entire length of the sequence. Preferably, all or most of the modified nucleotides are within the single-stranded loop region. In certain embodiments, the modified nucleotides are not limited to pyrimidines (i.e., any of G, U, A, or C), but the modifications are roughly evenly distributed along the entire length of the sequence. In yet another embodiment, no more than 4 consecutive polynucleotides are modified. In yet another embodiment, all nucleotides in the 3′-end stem region are modified. In a related embodiment, all nucleotides not in the guide sequence are modified.

In other embodiments, one or more chemical groups may be attached to the subject constructs, either covalently or non-covalently. The attachment point can be either at the ends (3′ or 5′ ends) or within the loop region. For example, either the 5′-end or the 3′-end (or both) of the subject constructs can be attached to DY547 or Cy3 florescent labeling. It is expected that modifying the 3′ end of the constructs will have less of an effect than modifying the 5′ end or both ends.

In other embodiments, the phosphodiester linkage may be modified. For example, as shown in FIG. 27, phosphorothioate (PS) linkages may be used. Such PS linkage may be incorporated only in the single-stranded loop region, only in the stem sense strand, in the stem sense strand plus 1, 2, 3, 4, 5, 6, or more nucleotides into the antisense strand, etc. In a related embodiment, a lipophilic linkage may be used to replace the phosphodiester linkage.

For these modified constructs, corresponding siRNA or rxRNA with the same or similar modification patterns may be used for activity control.

### Example X

### Conditions Favoring the Formation of Duplex Structures

The experiments described herein provide conditions that may favor the formation of duplex structures by two identical single-stranded polynucleotides (over the alternative mini hairpin structures formed by one single-stranded polynucleotide).

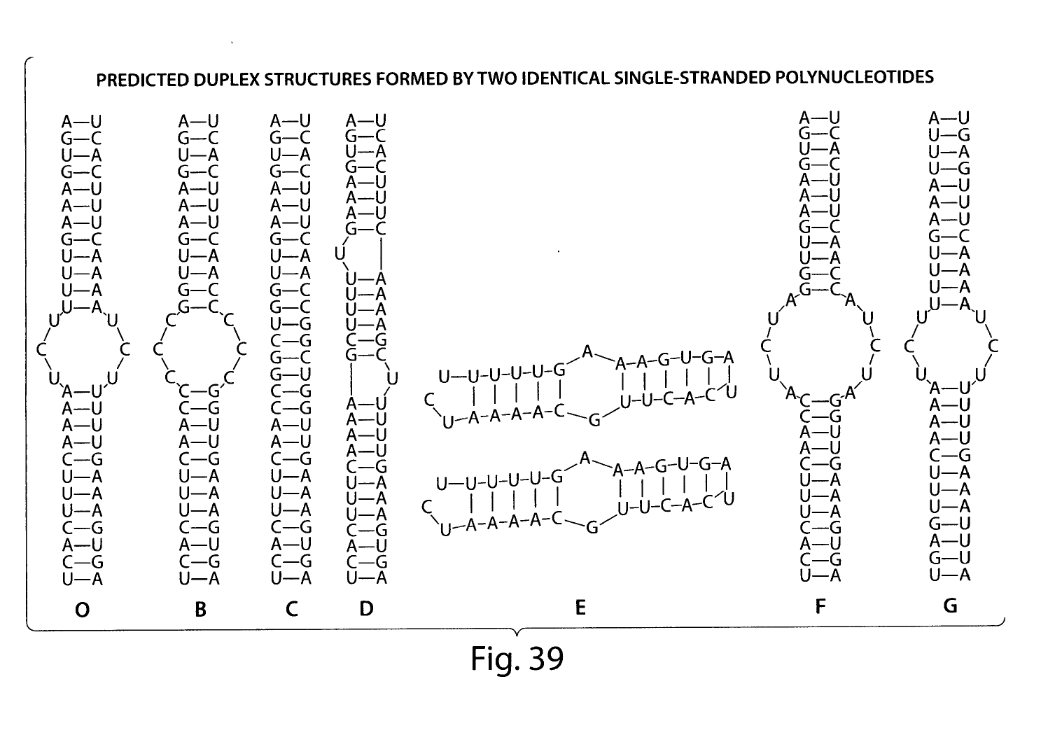

In this set of experiments, several similar constructs were prepared and tested over different conditions. The predicted structures of these constructs are shown in FIG. 39.

Construct “O” or the “original construct” refers to a desired double-stranded construct formed by two identical single-stranded palindromic polynucleotides. The construct is expected to have two identical 12-bp duplex regions flanking a loop in the middle, with 3 unpaired bases on each strand.

Construct “B” is otherwise identical to Construct O, except for slight differences in sequence at/around the bases of the duplex regions and the loop (“alteration of closing pair”).

Construct “C” is a perfect blunt-ended duplex, which does not have the loop structure, but is otherwise identical to Construct B in sequence outside the loop region.

Construct “D” is a non-favored loop without a closing pair. It is largely a duplex structure with two single base bulges, one on each strand. Its sequence is most similar to Construct O.

Construct “E” is a single-stranded circular construct with a single mismatch flanked by two 5-6 bp duplex regions. Construct E is identical in sequence to the single-stranded polynucleotide in Construct O, except for the mismatch nucleotide.

Construct “F” has a larger loop structure and loop sequence compared to Construct B, but is otherwise identical to Construct B.

Construct “G” has minor sequence changes compared to Construct O, such that one of its duplex stems has a lower Tₘ, than that of the other.

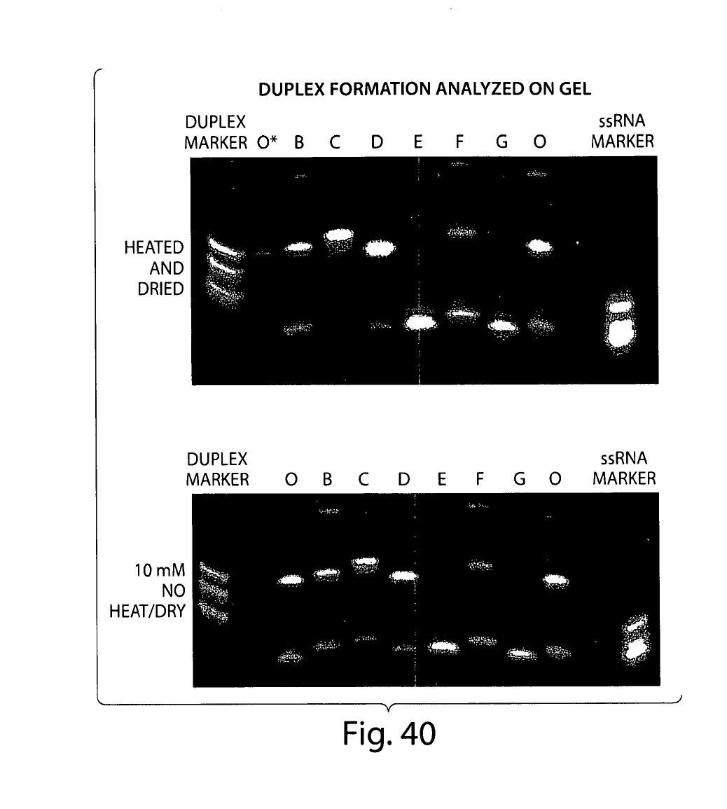

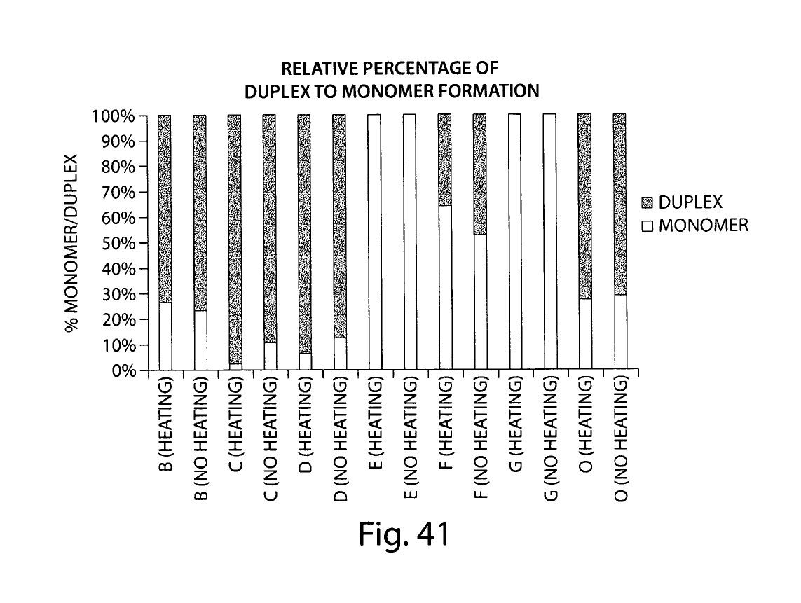

The various constructs above were prepared and reconstituted at 10 mM, in 3 M KCl, 30 mM HEPES buffer at pH 6.0. One set of samples were diluted directly in buffer and analyzed on gel. The other set of samples were first heated to 95 C for about 2 minutes, and then dried down on a Speed-vac at ambient temperature. The dried-down samples were then reconstituted in buffer and analyzed on gel. The results are shown in FIG. 40. The relative percentages of duplex and monomer were plotted in FIG. 41.

The results suggest that high starting concentrations (e.g., about 10 mM or more) of the polynucleotides and/or high salt buffer favor the formation of the duplex structure over the mini hairpin structure during reconstitution. It is expected that, during the dry-down procedure, the duplex is formed as the concentration of the polynucleotides increases with the decrease of water.

Heating prior to drying down may also promote duplex formation by, for example, promoting monomeric structures to open up and become available to form the duplex structure during the dry-down process. This effect, however, can be negligible in certain constructs.

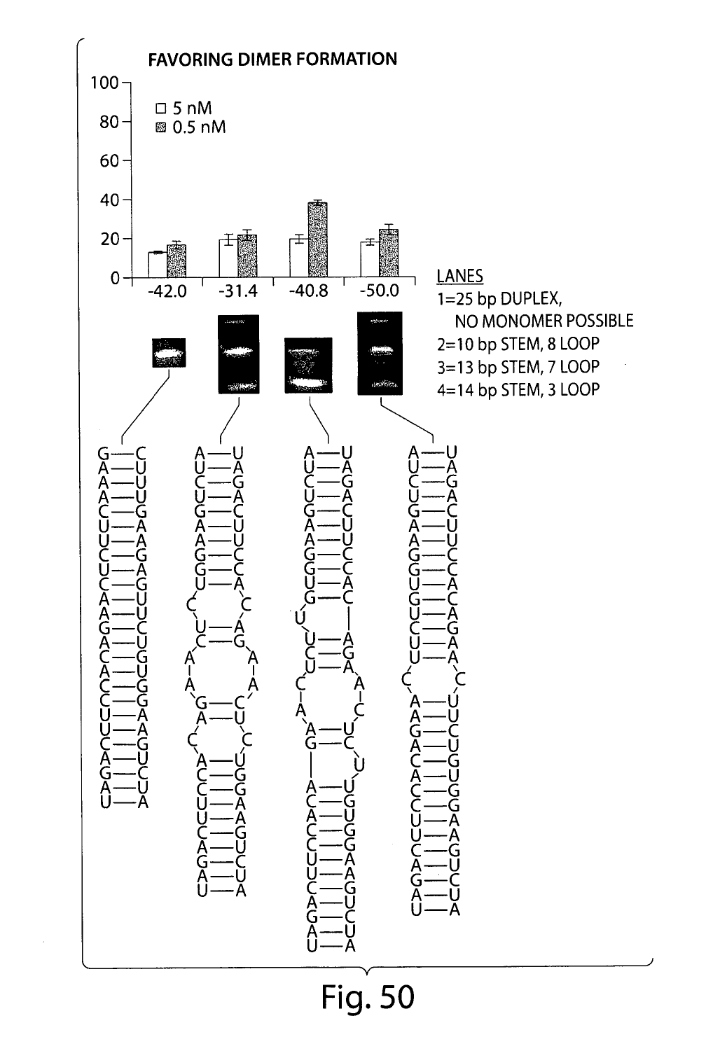

The results also suggest that certain structural features may help the formation of the duplex structure. For example, FIG. 50 illustrates additional solo-rxRNA designs that favor dimer formation. In addition, the size of the loop may affect the percentage of duplex constructs, with 3-base loop being more favorable than 5-base loop for duplex formation.

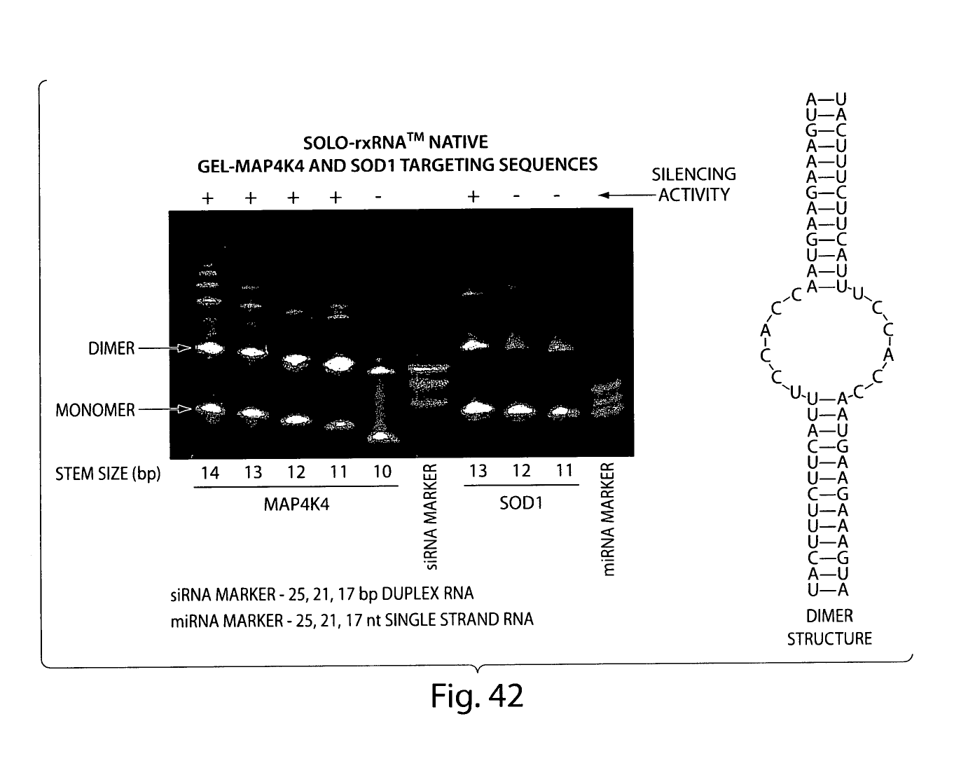

FIG. 42 suggests that the dimer or solo-rxRNA configuration may be the more active configuration in terms of RNAi activity. As shown in the gel image, diminished gene silencing activity seems to coincide with the disappearance of the solo-rxRNA conformation. This has been observed for both the MAP4K4 and SOD1 targeting sequences.

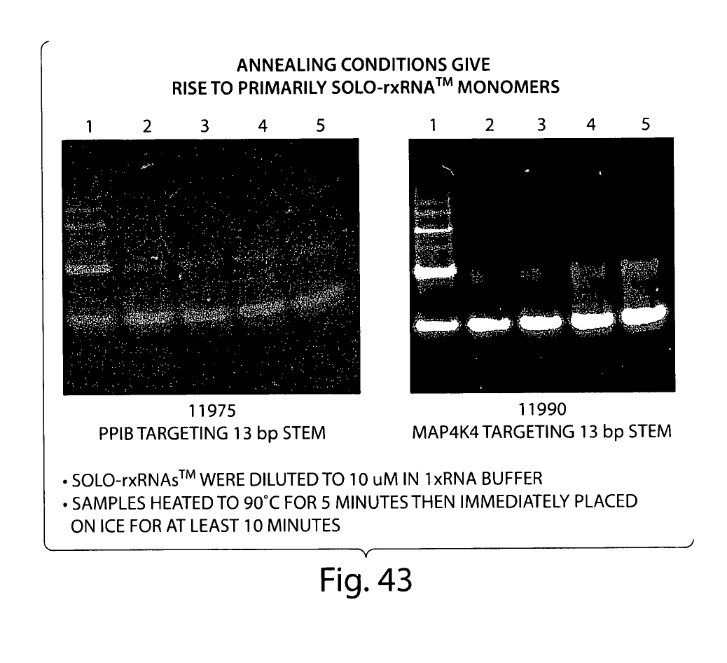

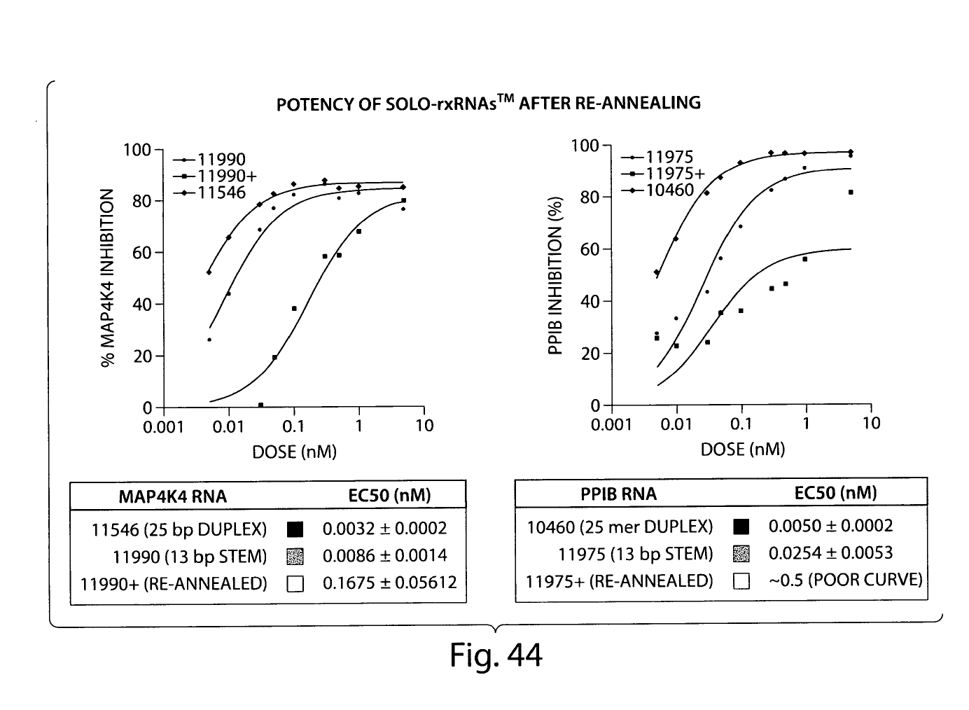

Consistent with this observation, Applicants have developed re-annealing conditions that preferentially generate the monomer (single-stranded) form, and showed that the monomer form is associated with much weaker RNAi activity. Specifically, the subject solo-rxRNA duplex can be diluted to 10 μM in 1×RNA buffer, heated to 90° C. for 5 minutes, then immediately placed on ice for at least 10 minutes to preferentially form the monomer. For both a PPIB targeting solo-rxRNA sequence and a MAP4K4 targeting solo-rxRNA sequence, FIG. 43 shows that the activity of the respective re-annealed monomer is much less compared to the corresponding solo-rxRNA duplex, as evidence by the increase in EC₅₀ values and the reduced peak inhibition % after re-annealing to monomer (FIG. 44).

### Example XI

### Dual Targeting Constructs

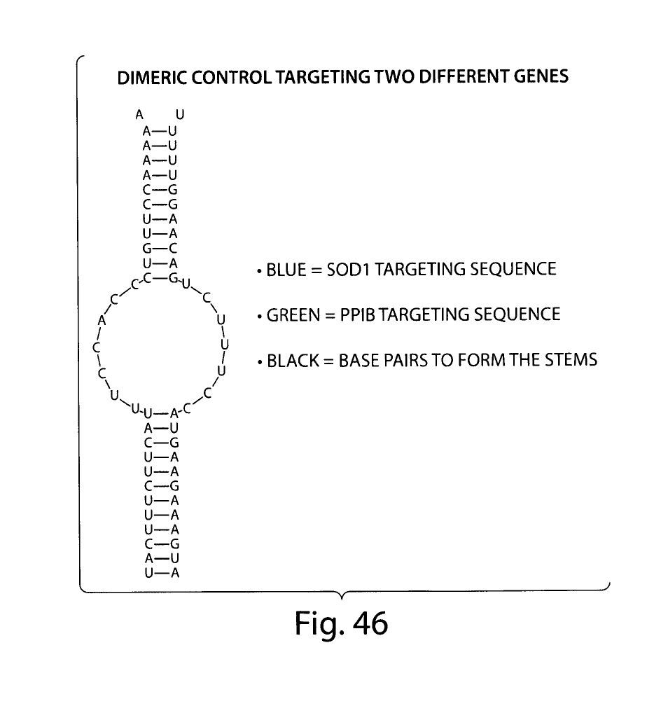

Applicants have also designed a dual targeting construct to simultaneously inhibit the activity of two unrelated target genes—SOD1 and PPIB. Similar constructs may also be generated to target different regions of the same target gene. An exemplary design is illustrated in FIG. 46, in which one strand targets the SOD1 gene, while the other strand targets the PPIB gene. Both guide sequences are 19 bases in length, and each comprises one 12-nucleotide stem sequence and a 7-nucleotide linker sequence in the loop region.

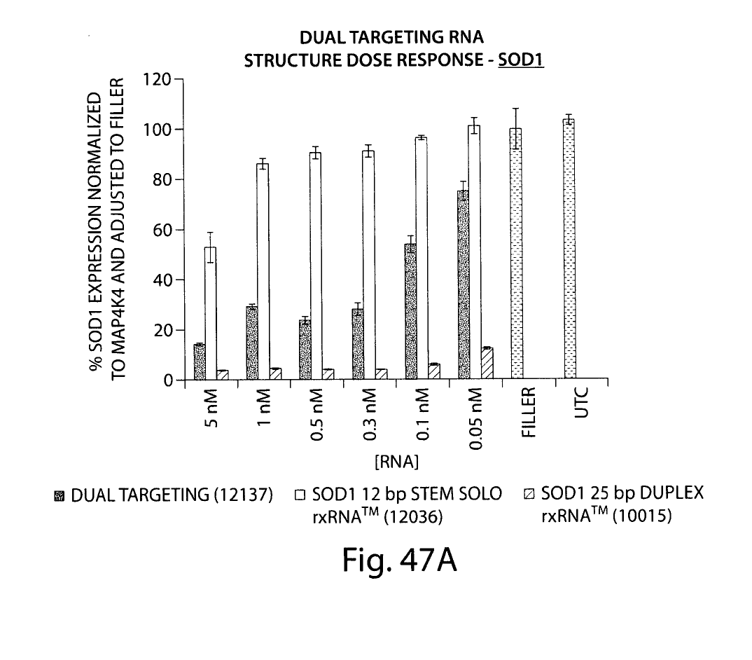

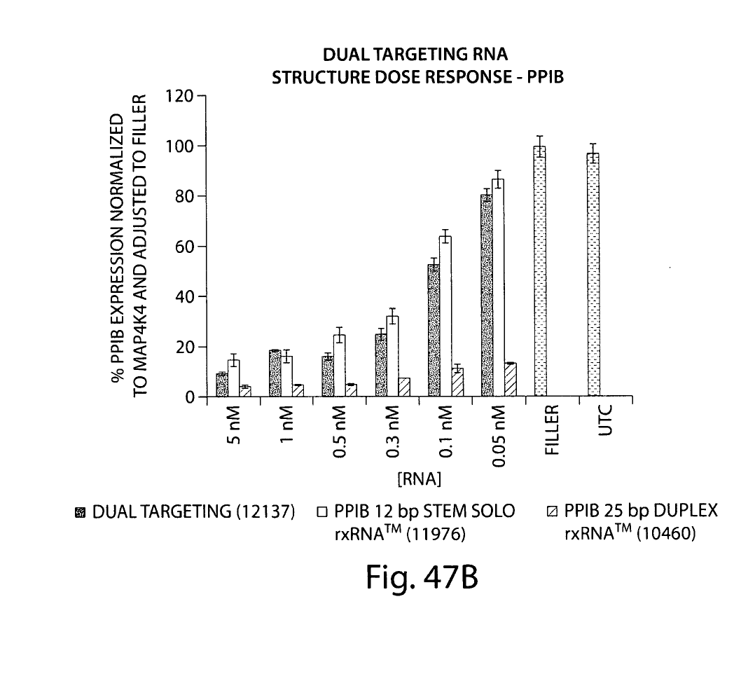

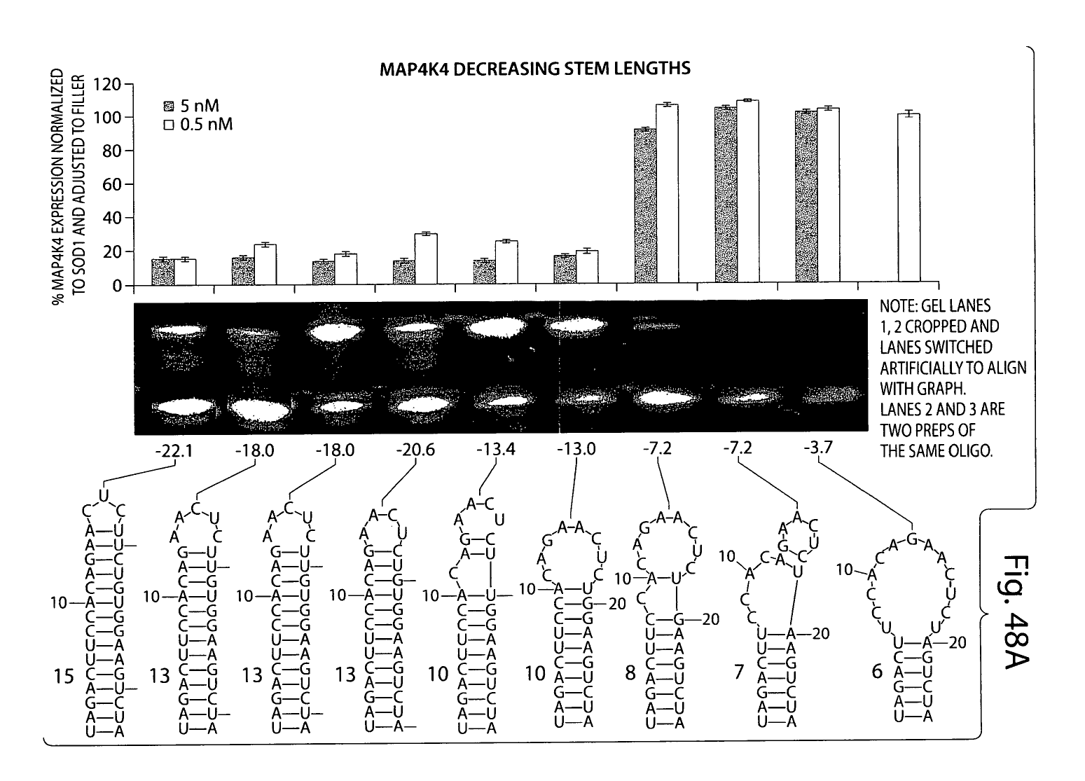

FIG. 47A shows that the dual-targeting construct appears to be more potent that the corresponding solo-rxRNA for SOD1. The dual-targeting construct inhibits SOD1 expression in a dose-dependent manner.

Similarly, FIG. 47B shows that the dual-targeting construct is at least as potent as the corresponding solo-rxRNA for PPIB. The dual-targeting construct inhibits PPIB expression in a dose-dependent manner.

### Example XII

### Dicer Cleavage and RISC Loading

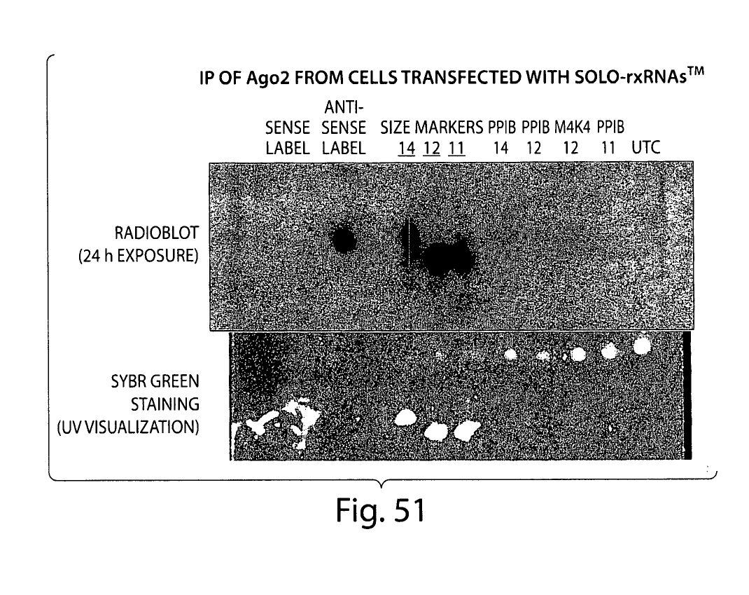

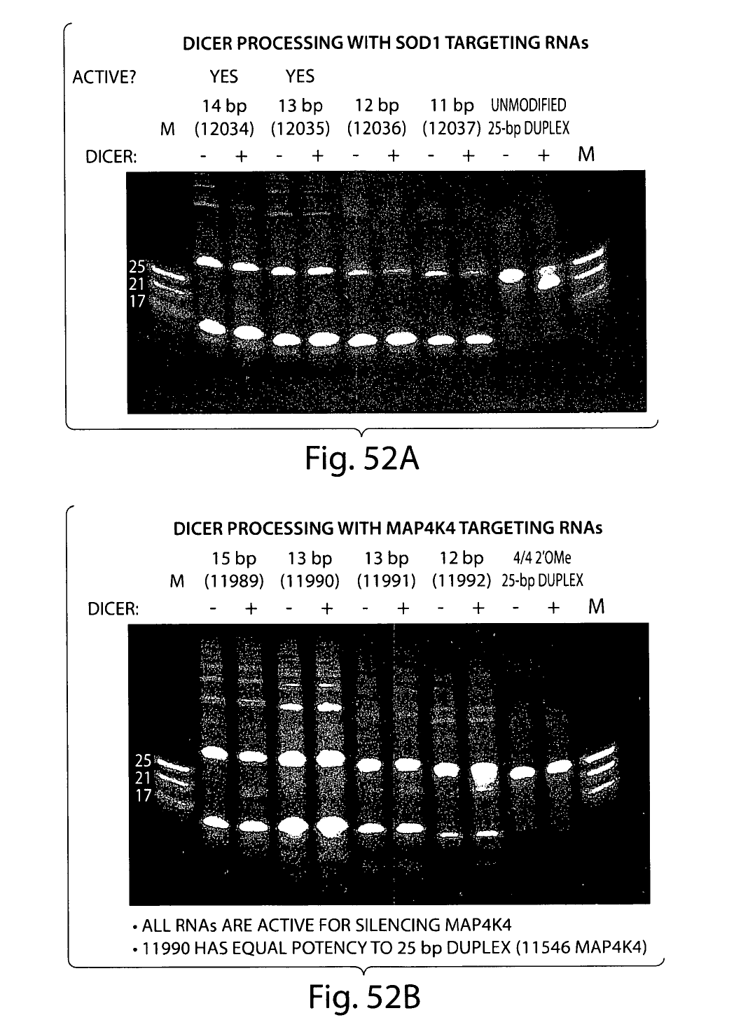

The solo-rxRNA constructs designed to specifically target MAP4K4 and SOD1 and shown to be effective herein have been examined to determine if they are processed by Dicer. As shown in FIGS. 52A and 52B, constructs demonstrated to be active in silencing SOD1 or MAP4K4 are resistant to Dicer cleavage. See FIGS. 15 and 13 which demonstrate activity of the specific constructs of SOD1 and MAP4K4 tested, respectively, in FIGS. 52A and 52B. Additionally, as shown in FIG. 51, cells transfected with various solo-rxRNA constructs were immunoprecipitated with Argonaute2 (Ago2) antibody. In such immunoprecipitated samples, the transfected solo-rxRNA constructs were detected, indicating that these constructs are efficiently loaded onto the RISC complex. These results show that Dicer processing is not necessary for RNAi activity or efficient RISC loading of the solo-rxRNA constructs.

### Example XIII

### Serum Stability

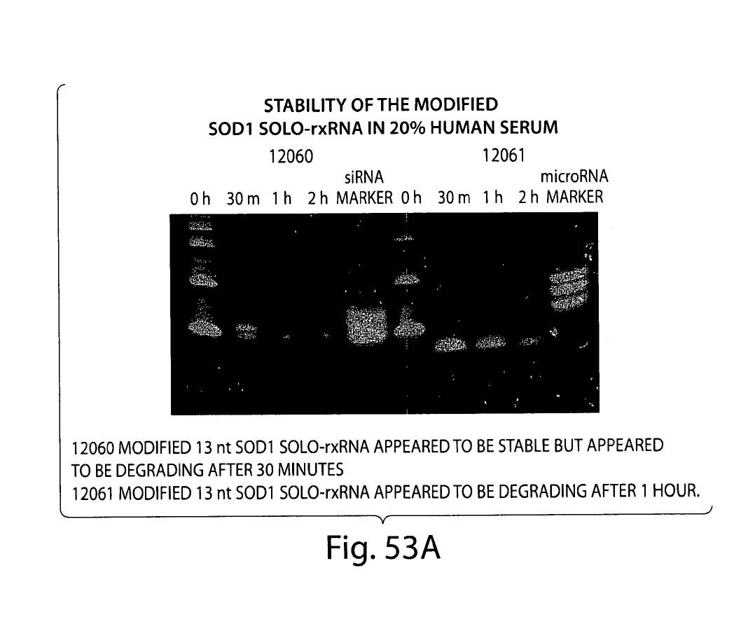

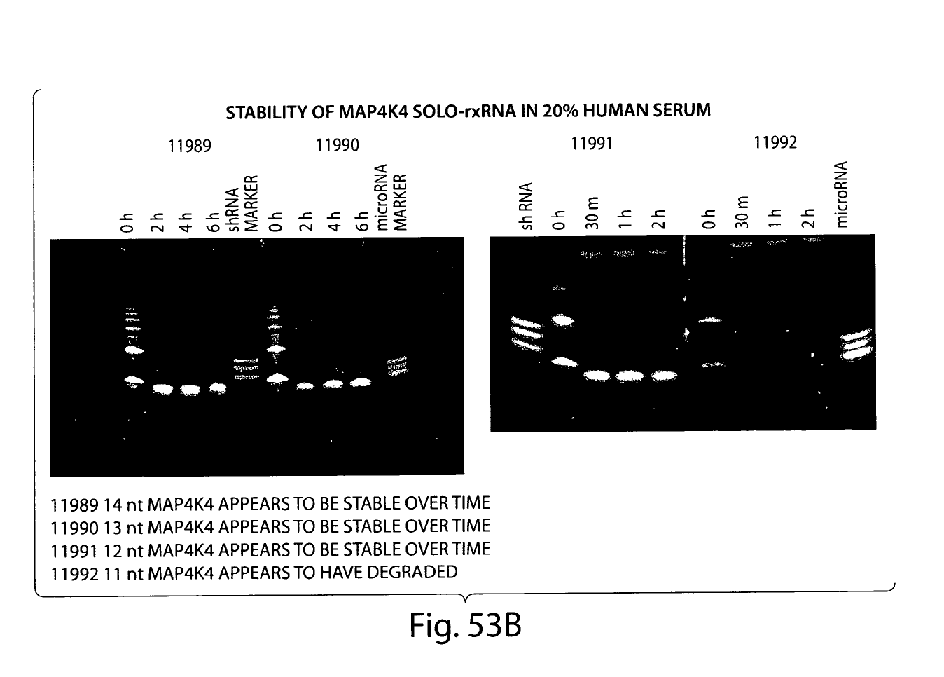

The serum stability of the subject solo-rxRNA constructs were tested in 20% human serum. FIG. 53A shows the stability of the modified SOD1 solo-rxRNA in 20% human serum. The modified 13-nt SOD1 solo-rxRNA (12060) was stable for at least 30 min., but appeared to be degrading after 30 minutes. The modified 13-nt SOD1 solo-rxRNA 12061 appeared to be degrading after 1 hour.

FIG. 53B shows the stability of MAP4K4 solo-rxRNA constructs in 20% human serum. It appears that solo-rxRNA constructs capable of forming stable stem regions, such as those with stems at least 12 nucleotides in length, are stable over the entire 6 hour testing period. In contrast, solo-rxRNA constructs with shorter stem regions, such as 11 nucleotides in this example, appears to be unstable.

### Example XIV

### Conditions Favoring the Silencing of Genes

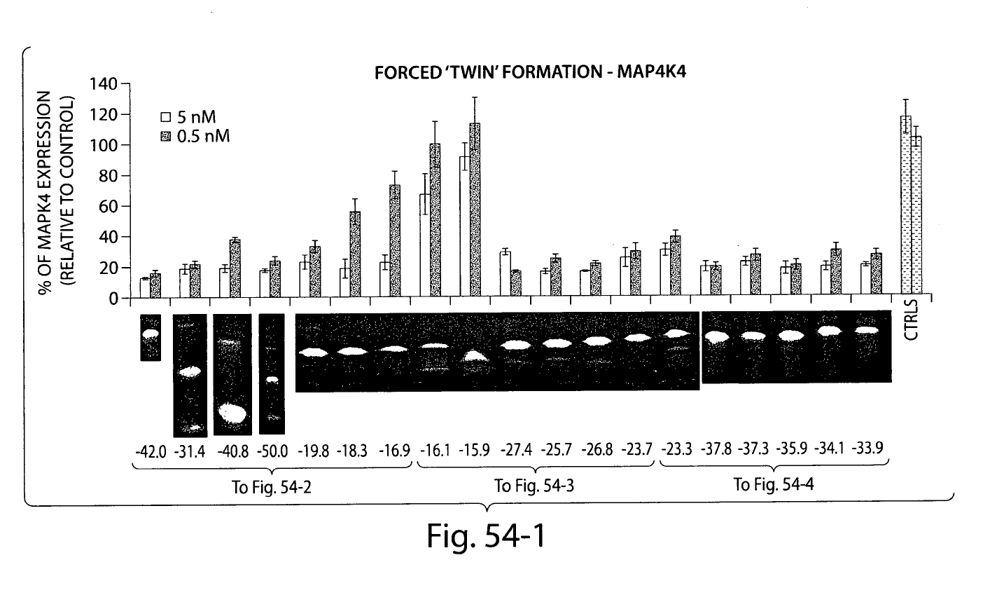

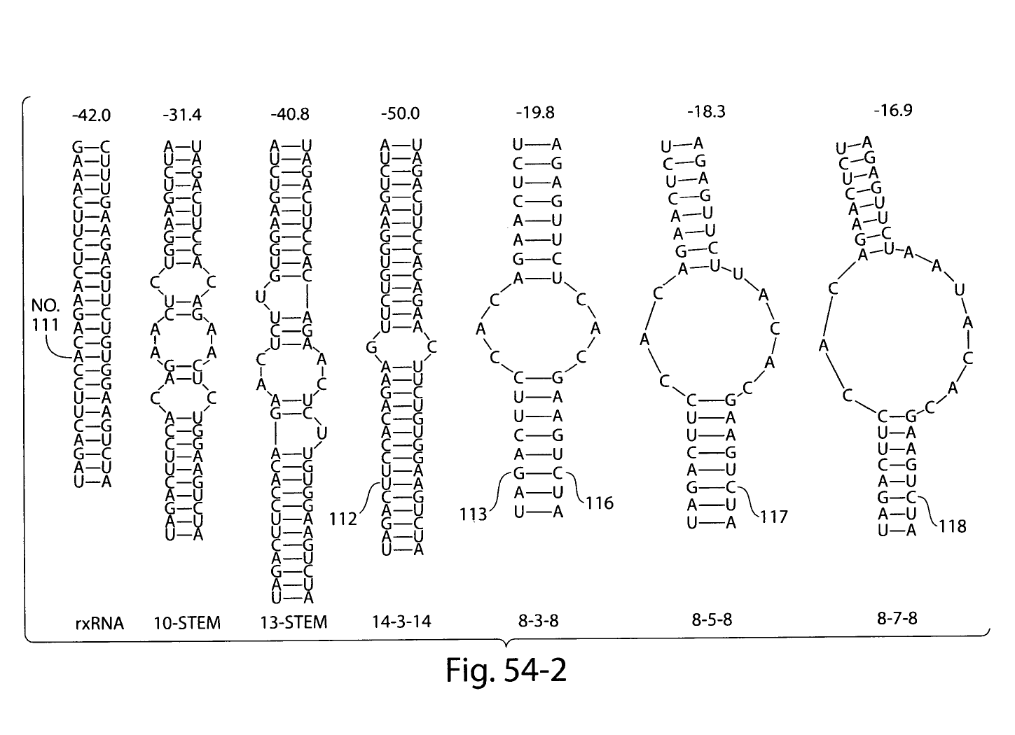

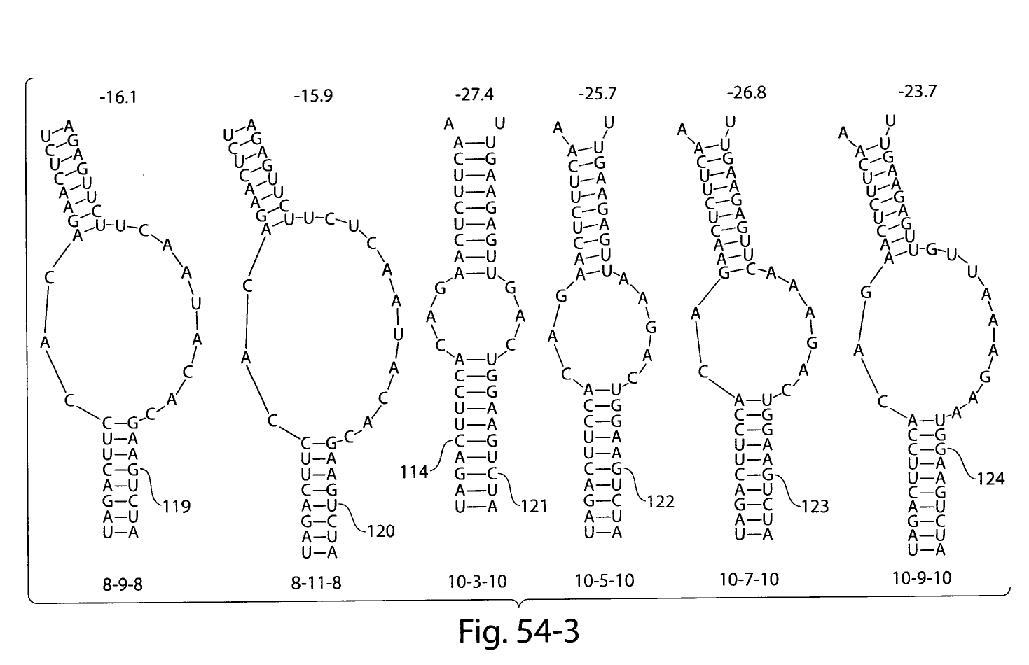

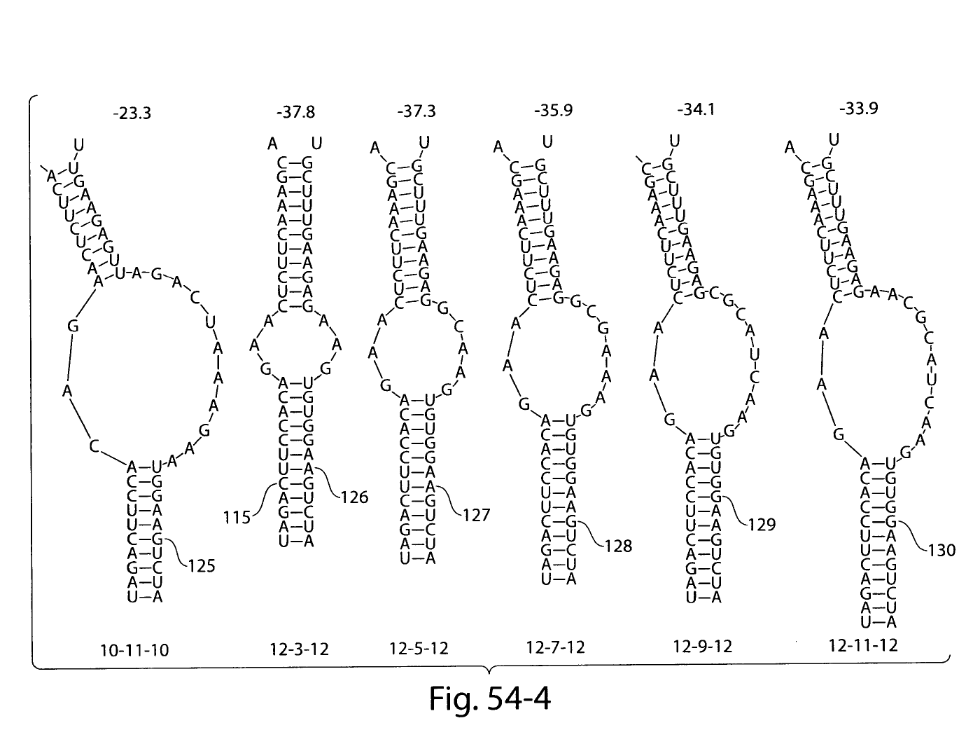

The experiments described herein demonstrate optimized structures of the invention for use in silencing gene expression. In this set of experiments, several similar constructs having stem and loop conformations of different length and compositions were prepared and tested over different conditions. The predicted structures of these constructs are shown at the bottom of FIG. 54.

The various constructs above were prepared and reconstituted at 10 mM, in 3 M KCl, 30 mM HEPES buffer at pH 6.0. The samples were diluted directly in buffer and analyzed on a gel. The results are shown in FIG. 54 in the form of a photograph of the gel and a bar graph. The Y axis refers to % Map4K expression relative to control. The results suggest that an optimal structure has central loops in the 3-4 nucleotide range.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Flames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

### EQUIVALENTS

Those skilled in the art will recognize, or be able to ascertain using no more than routine experimentation, many equivalents to the specific embodiments of the invention described herein. Such equivalents are intended to be encompassed by the following claims. The entire contents of all patents, published patent applications and other references cited herein are hereby expressly incorporated herein in their entireties by reference.

1. A polynucleotide construct comprising two identical single-stranded polynucleotides, wherein each single-stranded polynucleotide comprises a 5′-stem sequence having a 5′-end, a 3′-stem sequence having a 3′-end, and a linker sequence linking the 5′-stem sequence and the 3′-stem sequence, wherein:
(1) the 5′-stem sequence of a first single-stranded polynucleotide hybridizes with the 3′-stem sequence of a second single-stranded polynucleotide to form a first double-stranded stem region;
(2) the 5′-stem sequence of the second single-stranded polynucleotide hybridizes with the 3′-stem sequence of the first single-stranded polynucleotide to form a second double-stranded stem region; and,
(3) the linker sequences of the first and the second single-stranded polynucleotides form a loop or bulge connecting the first and the second double-stranded stem regions, wherein the 5′-stem sequence and at least a portion of the linker sequence form an antisense sequence complementary to a transcript of a target gene, wherein said polynucleotide construct mediates sequence-dependent gene silencing of expression of the target gene.

2. The polynucleotide construct of claim 1, wherein the 5′-stem sequence, the loop, and at least a portion of the 3′-stem sequence collectively form the antisense sequence complementary to the transcript of the target gene.

3. The polynucleotide construct of claim 1, wherein the antisense sequence is about 15-21 nucleotides in length, about 17-21 nucleotides in length, about 19-21 nucleotides in length, about 17-18 nucleotides in length or about 16-18 nucleotides in length.

4. The polynucleotide construct of claim 1, wherein each of the single-stranded polynucleotides is about 15-49 nucleotides in length, about 33-35 nucleotide in length, or about 25-27 nucleotides in length, and/or wherein each of the first and second double-stranded stem regions is less than about 21 base pairs in length, less than about 20 base pairs in length, about 5-15 base pairs in length, or about 11-14 base pairs in length.

5-8. (canceled)

9. The polynucleotide construct of claim 1, wherein each of the double-stranded regions is at least 8, 9, 10, 11 or 12 base pairs in length.

10. The polynucleotide construct of claim 1, wherein the linker sequence is 1, 2, 3, 4, 5, 6, 7, 8, 9, 10, 11, 12, 13, or 14 nucleotides in length.

11-14. (canceled)

15. The polynucleotide construct of claim 1, wherein at least one nucleotide is modified to improve resistance to nucleases, serum stability, target specificity, blood system circulation, tissue distribution, tissue penetration, cellular uptake, potency, and/or cell-permeability of the polynucleotide.

16. The polynucleotide construct of claim 15, wherein the modified nucleotides are modified on the sugar moiety, the base, and/or the phosphodiester linkage.

17-23. (canceled)

24. The polynucleotide construct of claim 15, wherein the modification is a 2′-O-alkyl or 2′-halo group.

25. The polynucleotide construct of claim 24, wherein the modification comprises:
(1) a 2′-O-methyl modification of one or more pyrimidine nucleotides (C or U);
(2) a 2′-O-methyl modification of one or more nucleotides within the loop;
(3) a 2′-O-methyl modification of at least 30% of all nucleotides;
(4) a 2′-O-methyl modification of all nucleotides in the 3′-end stem region;
(5) a 2′-O-methyl modification of all nucleotides 3′ to the loop;
(6) a hydrophobic modification of one or more bases, optionally wherein the hydrophobic modification comprises an isobutyl group;
(7) one or more phosphate modifications, optionally wherein the phosphate modifications are phosphorothioate modifications; and/or
(8) one or more 2′-fluoro modifications, optionally wherein at least one C or U nucleotide in positions 2-10 of the first single-stranded polynucleotide has a 2′-fluoro modification.

26-46. (canceled)

47. A pharmaceutical composition comprising the polynucleotide construct of claim 1, and a pharmaceutically acceptable salt, diluent, excipient, or carrier.

48. (canceled)

49. A method of inhibiting expression of a target gene with a polynucleotide construct of claim 1, wherein the polynucleotide construct mediates antisense sequence-dependent reduction in expression of the target gene.

50-63. (canceled)

64. A polynucleotide construct comprising a first single-stranded polynucleotide and a second single-stranded polynucleotide, each comprising a 5′-stem sequence having a 5′-end, a 3′-stem sequence having a 3′-end, and a linker sequence linking the 5′-stem sequence and the 3′-stem sequence, wherein:
(1) the 5′-stem sequence of the first single-stranded polynucleotide hybridizes with the 3′-stem sequence of the second single-stranded polynucleotide to form a first double-stranded stem region;
(2) the 5′-stem sequence of the second single-stranded polynucleotide hybridizes with the 3′-stem sequence of the first single-stranded polynucleotide to form a second double-stranded stem region; and,
(3) the linker sequences of the first and the second single-stranded polynucleotides form a loop or bulge connecting the first and the second double-stranded stem regions, wherein the loop is at least 3 nucleotides in length, wherein the 5′-stem sequence and at least a portion of the linker sequence for the first single-stranded polynucleotide form a first antisense sequence complementary to a transcript of a first target gene, and the 5′-stem sequence and at least a portion of the linker sequence for the second single-stranded polynucleotide form a second antisense sequence complementary to a transcript of a second target gene, and, wherein the polynucleotide construct mediates sequence-dependent gene silencing of expression of the first and second target genes.

65-76. (canceled)

77. A single-stranded polynucleotide of less than 35 nucleotides in length that forms a hairpin structure, wherein the hairpin includes a double-stranded stem and a single-stranded loop, the double-stranded stem having a 5′-stem sequence having a 5′-end, and a 3′-stem sequence having a 3′-end; and the 5′-stem sequence and at least a portion of the loop form an antisense sequence complementary to a transcript of a target gene, wherein the polynucleotide mediates sequence-dependent gene silencing of expression of the target gene.

78. The single-stranded polynucleotide of claim 77, wherein the 5′-stem sequence, the loop, and at least a portion of the 3′-stem sequence collectively form the antisense sequence complementary to the transcript of the target gene.

79-81. (canceled)

82. A method of treating a patient for a disease characterized by overexpression of a target gene, comprising administering to the patient a therapeutically effective amount of a polynucleotide construct of claim 1, wherein the polynucleotide construct mediates antisense sequence-dependent reduction in expression of the target gene.

83. (canceled)

84. The polynucleotide construct of claim 1, wherein the linker sequence of each single-stranded polynucleotide is 8 nucleotides in length, and wherein the 3′-end stem region of each single-stranded polynucleotide is highly modified with 2′-O-methyl modifications.

85. The polynucleotide construct of claim 64, wherein the 5′-stem sequence, the loop, and at least a portion of the 3′-stem sequence collectively form the antisense sequence complementary to the transcript of the target gene.

86. The polynucleotide construct of claim 64, wherein at least one nucleotide is modified and wherein the modification comprises:
(1) a 2′-O-methyl modification of one or more pyrimidine nucleotides (C or U);
(2) a 2′-O-methyl modification of one or more nucleotides within the loop;
(3) a 2′-O-methyl modification of at least 30% of all nucleotides;
(4) a 2′-O-methyl modification of all nucleotides in the 3′-end stem region;
(5) a 2′-O-methyl modification of all nucleotides 3′ to the loop;
(6) a hydrophobic modification of one or more bases, optionally wherein the hydrophobic modification comprises an isobutyl group;
(7) one or more phosphate modifications, optionally wherein the phosphate modifications are phosphorothioate modifications; and/or
(8) one or more 2′-fluoro modifications, optionally wherein at least one C or U nucleotide in positions 2-10 of the first single-stranded polynucleotide has a 2′-fluoro modification.

87. The polynucleotide construct of claim 64, wherein the linker sequence of each single-stranded polynucleotide is 8 nucleotides in length, and wherein the 3′-end stem region of each single-stranded polynucleotide is highly modified with 2′-O-methyl modifications.


SEQUENCE LISTING (130 sequences)

SEQ ID NO: 1 - RNA, 34 nt, Artificial sequence
  Synthetic
uuuuuggaacagucuuuccagacuguuccaaaaa

SEQ ID NO: 2 - RNA, 32 nt, Artificial sequence
  Synthetic
uuuuuggaacagucuuuccacuguuccaaaaa

SEQ ID NO: 3 - RNA, 30 nt, Artificial sequence
  Synthetic
uuuuuggaacagucuuuccuguuccaaaaa

SEQ ID NO: 4 - RNA, 28 nt, Artificial sequence
  Synthetic
uuuuuggaacagucuuunnuuccaaaaa

SEQ ID NO: 5 - RNA, 26 nt, Artificial sequence
  Synthetic
uuuuuggaacagucuunnnccaaaaa

SEQ ID NO: 6 - RNA, 49 nt, Artificial sequence
  Synthetic
aaaaaccuugucagaaagguucaagagaccuuucugacaagguuuuuuu

SEQ ID NO: 7 - RNA, 25 nt, Artificial sequence
  Synthetic
cucuucggaaagacuguuccaaaaa

SEQ ID NO: 8 - RNA, 34 nt, Artificial sequence; identical to SEQ ID NO: 1
  Synthetic

SEQ ID NO: 9 - RNA, 14 nt, Artificial sequence
  Synthetic
gacuguuccaaaaa

SEQ ID NO: 10 - RNA, 14 nt, Artificial sequence
  Synthetic
uuuuuggaacaguc

SEQ ID NO: 11 - RNA, 32 nt, Artificial sequence; identical to SEQ ID NO: 2
  Synthetic

SEQ ID NO: 12 - RNA, 13 nt, Artificial sequence
  Synthetic
acuguuccaaaaa

SEQ ID NO: 13 - RNA, 13 nt, Artificial sequence
  Synthetic
uuuuuggaacagu

SEQ ID NO: 14 - RNA, 30 nt, Artificial sequence; identical to SEQ ID NO: 3
  Synthetic

SEQ ID NO: 15 - RNA, 12 nt, Artificial sequence
  Synthetic
cuguuccaaaaa

SEQ ID NO: 16 - RNA, 12 nt, Artificial sequence
  Synthetic
uuuuuggaacag

SEQ ID NO: 17 - RNA, 28 nt, Artificial sequence
  Synthetic
uuuuuggaacagucuuunnuuccaaaaa

SEQ ID NO: 18 - RNA, 11 nt, Artificial sequence
  Synthetic
uguuccaaaaa

SEQ ID NO: 19 - RNA, 11 nt, Artificial sequence
  Synthetic
uuuuuggaaca

SEQ ID NO: 20 - RNA, 26 nt, Artificial sequence
  Synthetic
uuuuuggaacagucuunnnccaaaaa

SEQ ID NO: 21 - RNA, 10 nt, Artificial sequence
  Synthetic
guuccaaaaa

SEQ ID NO: 22 - RNA, 10 nt, Artificial sequence
  Synthetic
uuuuuggaac

SEQ ID NO: 23 - RNA, 25 nt, Artificial sequence
  Synthetic
cucuucggaaagacuguuccaaaaa

SEQ ID NO: 24 - RNA, 25 nt, Artificial sequence
  Synthetic
uuuuuggaacagucuuuccgaagag

SEQ ID NO: 25 - RNA, 47 nt, Artificial sequence
  Synthetic
uuuuuggaacagucuuuccuucaagagaggaaagacuguuccaaaaa

SEQ ID NO: 26 - RNA, 43 nt, Artificial sequence
  Synthetic
uuuuuggaacagucuuucccuuccggaaagacuguuccaaaaa

SEQ ID NO: 27 - RNA, 19 nt, Artificial sequence
  Synthetic
ggaaagacuguuccaaaaa

SEQ ID NO: 28 - RNA, 13 nt, Artificial sequence
  Synthetic
uuuuuggaacagu

SEQ ID NO: 29 - RNA, 34 nt, Artificial sequence
  Synthetic
uagacuuccacagaacucnuucuguggaagucua

SEQ ID NO: 30 - RNA, 34 nt, Artificial sequence
  Synthetic
guaucucuucauagccuuaacuaugaagagauac

SEQ ID NO: 31 - RNA, 32 nt, Artificial sequence
  Synthetic
uagacuuccacagaacucncuguggaagucua

SEQ ID NO: 32 - RNA, 32 nt, Artificial sequence
  Synthetic
guaucucuucauagccuuauaugaagagauac

SEQ ID NO: 33 - RNA, 30 nt, Artificial sequence
  Synthetic
uagacuuccacagaacucuguggaagucua

SEQ ID NO: 34 - RNA, 30 nt, Artificial sequence
  Synthetic
guaucucuucauagccuuaugaagagauac

SEQ ID NO: 35 - RNA, 28 nt, Artificial sequence
  Synthetic
uagacuuccacagaacunuggaagucua

SEQ ID NO: 36 - RNA, 28 nt, Artificial sequence
  Synthetic
guaucucuucauagccuunaagagauac

SEQ ID NO: 37 - RNA, 26 nt, Artificial sequence
  Synthetic
uagacuuccacagannunnaagucua

SEQ ID NO: 38 - RNA, 26 nt, Artificial sequence
  Synthetic
guaucucuucauagccnnagagauac

SEQ ID NO: 39 - RNA, 25 nt, Artificial sequence
  Synthetic
gcacucugauugacaaauacgauuu

SEQ ID NO: 40 - RNA, 25 nt, Artificial sequence
  Synthetic
aaaucguauuugucaaucagagugc

SEQ ID NO: 41 - RNA, 25 nt, Artificial sequence
  Synthetic
cuuugaagaguucuguggaagucua

SEQ ID NO: 42 - RNA, 25 nt, Artificial sequence
  Synthetic
uagacuuccacagaacucuucaaag

SEQ ID NO: 43 - RNA, 31 nt, Artificial sequence
  Synthetic
uagacuuccacagaacucuuguggaagucua

SEQ ID NO: 44 - RNA, 33 nt, Artificial sequence
  Synthetic
uagacuuccacagaacucuucuguggaagucua

SEQ ID NO: 45 - RNA, 31 nt, Artificial sequence; identical to SEQ ID NO: 43
  Synthetic

SEQ ID NO: 46 - RNA, 30 nt, Artificial sequence; identical to SEQ ID NO: 33
  Synthetic

SEQ ID NO: 47 - RNA, 29 nt, Artificial sequence
  Synthetic
uagacuuccacagaacucuuggaagucua

SEQ ID NO: 48 - RNA, 28 nt, Artificial sequence
  Synthetic
uagacuuccacagaacucuggaagucua

SEQ ID NO: 49 - RNA, 27 nt, Artificial sequence
  Synthetic
uagacuuccacagaacucugaagucua

SEQ ID NO: 50 - RNA, 26 nt, Artificial sequence
  Synthetic
uagacuuccacagaacucuaagucua

SEQ ID NO: 51 - RNA, 25 nt, Artificial sequence
  Synthetic
uagacuuccacagaacucuagucua

SEQ ID NO: 52 - RNA, 32 nt, Artificial sequence
  Synthetic
uuacacgauggaauuugcuuuccaucguguaa

SEQ ID NO: 53 - RNA, 30 nt, Artificial sequence
  Synthetic
uuacacgauggaauuugcuccaucguguaa

SEQ ID NO: 54 - RNA, 25 nt, Artificial sequence
  Synthetic
aaaaacagcaaauuccaucguguaa

SEQ ID NO: 55 - RNA, 25 nt, Artificial sequence
  Synthetic
uuacacgauggaauuugcuguuuuu

SEQ ID NO: 56 - RNA, 34 nt, Artificial sequence
  Synthetic
uacuuucuucauuuccacccaaaugaagaaagua

SEQ ID NO: 57 - RNA, 32 nt, Artificial sequence
  Synthetic
uacuuucuucauuuccaccaaugaagaaagua

SEQ ID NO: 58 - RNA, 30 nt, Artificial sequence
  Synthetic
uacuuucuucauuuccacnugaagaaagua

SEQ ID NO: 59 - RNA, 28 nt, Artificial sequence
  Synthetic
uacuuucuucauuuccangaagaaagua

SEQ ID NO: 60 - RNA, 26 nt, Artificial sequence
  Synthetic
uacuuucuucauuuccnaagaaagua

SEQ ID NO: 61 - RNA, 24 nt, Artificial sequence
  Synthetic
uacuuucuucauuucnagaaagua

SEQ ID NO: 62 - RNA, 22 nt, Artificial sequence
  Synthetic
uacuuucuucauuunnaaagua

SEQ ID NO: 63 - RNA, 20 nt, Artificial sequence
  Synthetic
uacuuucuucauunnnagua

SEQ ID NO: 64 - RNA, 25 nt, Artificial sequence
  Synthetic
ggcaaagguggaaaugaagaaagua

SEQ ID NO: 65 - RNA, 25 nt, Artificial sequence
  Synthetic
uacuuucuucauuuccaccuuugcc

SEQ ID NO: 66 - RNA, 32 nt, Artificial sequence
  Synthetic
caacaugccucucuucaucgagaggcauguug

SEQ ID NO: 67 - RNA, 25 nt, Artificial sequence
  Synthetic
ccaaaggaugaagagaggcauguug

SEQ ID NO: 68 - RNA, 25 nt, Artificial sequence
  Synthetic
caacaugccucucuucauccuuugg

SEQ ID NO: 69 - RNA, 32 nt, Artificial sequence
  Synthetic
uaaagugaggaccugcacugguccucacuuua

SEQ ID NO: 70 - RNA, 25 nt, Artificial sequence
  Synthetic
uguaccagugcagguccucacuuua

SEQ ID NO: 71 - RNA, 25 nt, Artificial sequence
  Synthetic
uaaagugaggaccugcacugguaca

SEQ ID NO: 72 - RNA, 32 nt, Artificial sequence
  Synthetic
ucagcagucacauugcccaaugugacugcuga

SEQ ID NO: 73 - RNA, 25 nt, Artificial sequence
  Synthetic
gagacuugggcaaugugacugcuga

SEQ ID NO: 74 - RNA, 25 nt, Artificial sequence
  Synthetic
ucagcagucacauugcccaagucuc

SEQ ID NO: 75 - RNA, 32 nt, Artificial sequence
  Synthetic
cagaaucuucaauagacacauugaagauucug

SEQ ID NO: 76 - RNA, 25 nt, Artificial sequence
  Synthetic
gccgaugugucuauugaagauucug

SEQ ID NO: 77 - RNA, 25 nt, Artificial sequence
  Synthetic
cagaaucuucaauagacacaucggc

SEQ ID NO: 78 - RNA, 32 nt, Artificial sequence
  Synthetic
uguacuuucuucauuuccaugaagaaaguaca

SEQ ID NO: 79 - RNA, 25 nt, Artificial sequence
  Synthetic
caaagguggaaaugaagaaaguaca

SEQ ID NO: 80 - RNA, 25 nt, Artificial sequence
  Synthetic
uguacuuucuucauuuccaccuuug

SEQ ID NO: 81 - RNA, 32 nt, Artificial sequence
  Synthetic
aacaugccucucuucauccagagaggcauguu

SEQ ID NO: 82 - RNA, 25 nt, Artificial sequence
  Synthetic
gccaaaggaugaagagaggcauguu

SEQ ID NO: 83 - RNA, 25 nt, Artificial sequence
  Synthetic
aacaugccucucuucauccuuuggc

SEQ ID NO: 84 - RNA, 32 nt, Artificial sequence
  Synthetic
uccaacaugccucucuucagaggcauguugga

SEQ ID NO: 85 - RNA, 25 nt, Artificial sequence
  Synthetic
aaaggaugaagagaggcauguugga

SEQ ID NO: 86 - RNA, 25 nt, Artificial sequence
  Synthetic
uccaacaugccucucuucauccuuu

SEQ ID NO: 87 - RNA, 32 nt, Artificial sequence
  Synthetic
uucauuuccaccuuugcccagguggaaaugaa

SEQ ID NO: 88 - RNA, 25 nt, Artificial sequence
  Synthetic
ugacuugggcaaagguggaaaugaa

SEQ ID NO: 89 - RNA, 25 nt, Artificial sequence
  Synthetic
uucauuuccaccuuugcccaaguca

SEQ ID NO: 90 - RNA, 32 nt, Artificial sequence
  Synthetic
ucuccaacaugccucucuuggcauguuggaga

SEQ ID NO: 91 - RNA, 25 nt, Artificial sequence
  Synthetic
aggaugaagagaggcauguuggaga

SEQ ID NO: 92 - RNA, 25 nt, Artificial sequence
  Synthetic
ucuccaacaugccucucuucauccu

SEQ ID NO: 93 - RNA, 32 nt, Artificial sequence
  Synthetic
gauuaaagugaggaccugcccucacuuuaauc

SEQ ID NO: 94 - RNA, 25 nt, Artificial sequence
  Synthetic
accagugcagguccucacuuuaauc

SEQ ID NO: 95 - RNA, 25 nt, Artificial sequence
  Synthetic
gauuaaagugaggaccugcacuggu

SEQ ID NO: 96 - RNA, 32 nt, Artificial sequence
  Synthetic
uggcccaccguguuuucugacacggugggcca

SEQ ID NO: 97 - RNA, 25 nt, Artificial sequence
  Synthetic
ucuauccagaaaacacggugggcca

SEQ ID NO: 98 - RNA, 25 nt, Artificial sequence
  Synthetic
uggcccaccguguuuucuggauaga

SEQ ID NO: 99 - RNA, 32 nt, Artificial sequence
  Synthetic
cgaaauugaugaugcccugaucaucaauuucg

SEQ ID NO: 100 - RNA, 25 nt, Artificial sequence
  Synthetic
ccagugcagggcaucaucaauuucg

SEQ ID NO: 101 - RNA, 25 nt, Artificial sequence
  Synthetic
cgaaauugaugaugcccugcacugg

SEQ ID NO: 102 - RNA, 32 nt, Artificial sequence
  Synthetic
acaccuucacugguccauuccagugaaggugu

SEQ ID NO: 103 - RNA, 25 nt, Artificial sequence
  Synthetic
gaaaguaauggaccagugaaggugu

SEQ ID NO: 104 - RNA, 25 nt, Artificial sequence
  Synthetic
acaccuucacugguccauuacuuuc

SEQ ID NO: 105 - RNA, 32 nt, Artificial sequence
  Synthetic
aucuucaauagacacaucggucuauugaagau

SEQ ID NO: 106 - RNA, 25 nt, Artificial sequence
  Synthetic
uguggccgaugugucuauugaagau

SEQ ID NO: 107 - RNA, 25 nt, Artificial sequence
  Synthetic
aucuucaauagacacaucggccaca

SEQ ID NO: 108 - RNA, 32 nt, Artificial sequence
  Synthetic
uuugucagcagucacauuggacugcugacaaa

SEQ ID NO: 109 - RNA, 25 nt, Artificial sequence
  Synthetic
cuugggcaaugugacugcugacaaa

SEQ ID NO: 110 - RNA, 25 nt, Artificial sequence
  Synthetic
uuugucagcagucacauugcccaag

SEQ ID NO: 111 - RNA, 25 nt, Artificial sequence
  Synthetic
uagacuuccacagaacucuucaaag

SEQ ID NO: 112 - RNA, 31 nt, Artificial sequence
  Synthetic
uagacuuccacagaacuucuguggaagucua

SEQ ID NO: 113 - RNA, 19 nt, Artificial sequence
  Synthetic
uagacuuccacagaacucu

SEQ ID NO: 114 - RNA, 23 nt, Artificial sequence
  Synthetic
uagacuuccacagaacucuucaa

SEQ ID NO: 115 - RNA, 27 nt, Artificial sequence
  Synthetic
uagacuuccacagaacucuucaaagca

SEQ ID NO: 116 - RNA, 19 nt, Artificial sequence
  Synthetic
agaguucucacgaagucua

SEQ ID NO: 117 - RNA, 21 nt, Artificial sequence
  Synthetic
agaguucuuacacgaagucua

SEQ ID NO: 118 - RNA, 23 nt, Artificial sequence
  Synthetic
agaguucuaauacacgaagucua

SEQ ID NO: 119 - RNA, 25 nt, Artificial sequence
  Synthetic
agaguucuucaauacacgaagucua

SEQ ID NO: 120 - RNA, 27 nt, Artificial sequence
  Synthetic
agaguucuucucaauacacgaagucua

SEQ ID NO: 121 - RNA, 23 nt, Artificial sequence
  Synthetic
uugaagaguugacuggaagucua

SEQ ID NO: 122 - RNA, 25 nt, Artificial sequence
  Synthetic
uugaagaguuaagacuggaagucua

SEQ ID NO: 123 - RNA, 27 nt, Artificial sequence
  Synthetic
uugaagaguucaaagacuggaagucua

SEQ ID NO: 124 - RNA, 29 nt, Artificial sequence
  Synthetic
uugaagaguuguuaaagaauggaagucua

SEQ ID NO: 125 - RNA, 31 nt, Artificial sequence
  Synthetic
uugaagaguuagacuaaagaauggaagucua

SEQ ID NO: 126 - RNA, 27 nt, Artificial sequence
  Synthetic
ugcuuugaagagaaguguggaagucua

SEQ ID NO: 127 - RNA, 29 nt, Artificial sequence
  Synthetic
ugcuuugaagaggcaaguguggaagucua

SEQ ID NO: 128 - RNA, 30 nt, Artificial sequence
  Synthetic
ugcuuugaagaggcgaaguguggaagucua

SEQ ID NO: 129 - RNA, 33 nt, Artificial sequence
  Synthetic
ugcuuugaagagcgcaucaaguguggaagucua

SEQ ID NO: 130 - RNA, 35 nt, Artificial sequence
  Synthetic
ugcuuugaagagaacgcaucaaguguggaagucua